(19) United States
(12) Patent Application Publication
(10) Pub. No.: US 2025/0230511 A1
(43) Pub. Date: Jul. 17, 2025

(54) METHODS AND REAGENTS FOR AMPLIFYING VIRAL VECTOR NUCLEIC ACID PRODUCTS

(71) Applicant: Sana Biotechnology, Inc., Seattle, WA (US)

(72) Inventors: Andrew P. TUCKER, Seattle, WA (US); Semih U. TAREEN, Seattle, WA (US); Hallee A. WRIGHT, Seattle, WA (US)

(73) Assignee: Sana Biotechnology, Inc., Seattle, WA (US)

(21) Appl. No.: 18/705,410

(22) PCT Filed: Oct. 28, 2022
(86) PCT No.: PCT/US2022/078938

§ 371 Date: Apr. 26, 2024

Related U.S. Application Data
(60) Provisional application No. 63/273,836, filed on Oct. 29, 2021.
(60) Provisional application No. 63/341,903, filed on May 13, 2022.

(51) Int. Cl. C12Q 1/70; C12Q 1/6806; C12Q 1/6809; C12Q 1/6851

(52) U.S. Cl. CPC C12Q 1/703; C12Q 1/6806; C12Q 1/6809; C12Q 1/6851

(57) ABSTRACT

Provided herein are methods to amplify reverse-transcriptase dependent viral vector nucleic acid products, methods for determining viral vector copy number, and reagents including primers and probes for use in the methods.

### CROSS-REFERENCE TO RELATED APPLICATIONS

The present application claims priority from U.S. Provisional Application No. 63/273,836 filed Oct. 29, 2021, entitled “Methods and Reagents for Amplifying Viral Vector Nucleic Acid Products”, and from U.S. Provisional Application No. 63/341,903 filed on May 13, 2022, entitled “Methods and Reagents for Amplifying Viral Vector Nucleic Acid Products”, the contents of each of which are incorporated by reference in their entirety.

### INCORPORATION BY REFERENCE OF SEQUENCE LISTING

The present application is being filed along with a Sequence Listing in electronic format. The Sequence Listing is provided as a file entitled 18615_2005140_SeqList.XML, created Oct. 26, 2022, which is 62,588 bytes in size. The information in the electronic format of the Sequence Listing is incorporated by reference in its entirety.

### FIELD

The present disclosure provides methods to amplify reverse-transcriptase dependent viral vector nucleic acid products, methods for determining viral vector copy number, and reagents including primers and probes for use in the methods.

### SUMMARY

Provided herein is a method of amplifying a reverse-transcribed amplicon, the method comprising incubating DNA from a sample with (i) a forward oligonucleotide primer and a reverse oligonucleotide primer, wherein the primers are specific for a region of reverse-transcribed viral DNA of a self-inactivating (SIN) viral vector, and (ii) a DNA polymerase; and the incubating is under conditions sufficient for amplification of a reverse-transcriptase-dependent amplicon if the amplicon is present in the sample. Also provided herein is a method of detecting a reverse-transcribed amplicon, the method comprising (a) incubating DNA from a sample with (i) a forward oligonucleotide primer and a reverse oligonucleotide primer, wherein the primers are specific for a region of reverse-transcribed viral DNA of a self-inactivating (SIN) viral vector, and (ii) a DNA polymerase; and the incubating is under conditions sufficient for amplification of a reverse-transcriptase-dependent amplicon if the amplicon is present in the sample; and (b) detecting the generated reverse-transcriptase-dependent amplicon.

In some of any embodiments, the reverse-transcribed amplicon is reverse-transcriptase dependent. In some of any embodiments, the incubation is performed by polymerase chain reaction (PCR). In some of any embodiments, the PCR is quantitative PCR.

In some of any embodiments, the incubating is further with an oligonucleotide probe specific for the region of the reverse-transcribed viral DNA, wherein the oligonucleotide probe comprises a detectable moiety. In some of any embodiments, the method further comprises detecting a signal from the detectable moiety. In some of any embodiments, the method further comprises quantifying an amount of the generated reverse-transcriptase-dependent amplicon from the detected signal.

In some of any embodiments, the sample is known or suspected of containing viral nucleic acid from the SIN viral vector. In some of any embodiments, the sample further comprises a known positive control. In some embodiments, the positive control is a viral nucleic acid.

Provided herein is a method of quantifying a reverse-transcribed amplicon in a sample, the method comprising (a) performing quantitative PCR of DNA from a sample known or suspected of containing viral nucleic acid from a self-inactivating (SIN) viral vector by incubation of DNA from the sample with (i) a forward oligonucleotide primer and a reverse oligonucleotide primer, wherein the primers are specific for a region of reverse-transcribed viral DNA from the SIN viral vector, (ii) an oligonucleotide probe specific for the region of the reverse-transcribed viral DNA, wherein the oligonucleotide probe comprises a detectable moiety; and (iii) a DNA polymerase, wherein the quantitative PCR is performed under conditions sufficient for amplification of a reverse-transcriptase-dependent amplicon if the amplicon is present in the sample; and (b) quantifying an amount of the generated reverse-transcriptase-dependent amplicon by detection of a detectable signal from the detectable moiety.

Also provided herein is a method of determining viral vector copy number in a sample, the method comprising (a) performing quantitative PCR of DNA from a sample known or suspected of containing a viral nucleic acid from a self-inactivating viral vector by incubation of DNA from the sample with (i) a forward oligonucleotide primer and a reverse oligonucleotide primer, wherein the primers are specific for a regions of reverse-transcribed viral DNA from the SIN viral vector, (ii) an oligonucleotide probe specific for the region of the reverse-transcribed viral DNA, wherein the oligonucleotide probe comprises a detectable moiety, and (iii) a DNA polymerase, wherein the quantitative PCR is performed under conditions sufficient for amplification of a reverse-transcriptase-dependent amplicon if the amplicon is present in the sample; (b) quantifying an amount of the generated reverse-transcriptase-dependent amplicon by detection of a detectable signal from the detectable moiety; and (c) determining viral vector copy number in the sample.

In some of any embodiments, the reverse-transcribed amplicon is reverse-transcription dependent. In some of any embodiments, the quantitative PCR is real-time PCR. In some of any embodiments, the quantitative PCR is a digital PCR amplification. In some of any embodiments, the quantitative PCR is digital droplet PCR (ddPCR). In some of any embodiments, the SIN viral vector is a retroviral vector. In some of any embodiments, the SIN viral vector is a gamma-retroviral vector. In some of any embodiments, the retroviral vector is a lentiviral vector. In some of any embodiments, the lentiviral vector is derived from HIV-1.

In some of any embodiments, the forward oligonucleotide primer comprises a contiguous sequence of nucleotides of the plus strand of a first region that is within the deleted U3 (delU3) of the reverse-transcribed SIN viral vector, and the reverse oligonucleotide primer comprises a contiguous sequence of nucleotides of the minus strand of a second region downstream of the first region in the reverse-transcribed viral vector DNA to make a replicable reverse-transcriptase-dependent amplicon.

In some of any embodiments, the reverse-transcriptase-dependent amplicon is 50 base pairs to 500 base pairs in size. In some of any embodiments, the reverse-transcriptase-dependent amplicon is 100 base pairs to 200 base pairs in size. In some of any embodiments, the first region comprises a U3 attachment sequence.

In some of any embodiments, the first region comprises a sequence set forth in any one of SEQ ID NOS: 46-48. In some of any embodiments, the forward primer comprises at least a contiguous portion of any one of SEQ ID NOS: 46-48. In some of any embodiments, the first region comprises the sequence set forth in any one of SEQ ID NOS: 1 and 14-28 or a sequence having at least or about 85, 90, 91, 92, 93, 94, 95, 96, 97, 98, or 99% sequence identity to any one of SEQ ID NOS: 1 and 14-28. In some of any embodiments, the first region is set forth in SEQ ID NO:1.

In some of any embodiments, the second region comprises at least a portion of a primer binding site (PBS), an IFN-stimulated response element (ISRE), and/or a Psi (Ψ) packaging sequence of the SIN viral vector. In some of any embodiments, the second region comprises the sequence set forth in any one of SEQ ID NOS: 2 and 29-45 or a sequence having at least or about 85, 90, 91, 92, 93, 94, 95, 96, 97, 98, or 99% sequence identity to any one of SEQ ID NOS: 2 and 29-45.

In some of any embodiments, (i) the forward primer comprises a contiguous sequence of nucleotides within the sequence set forth in SEQ ID NO:1 or a sequence having at least or about 85, 90, 91, 92, 93, 94, 95, 96, 97, 98, or 99% sequence identity thereto, and the reverse primer comprises a contiguous sequence of nucleotides within the sequence set forth in SEQ ID NO:2 or a sequence having at least or about 85, 90, 91, 92, 93, 94, 95, 96, 97, 98, or 99% sequence identity thereto; (ii) the forward primer comprises a contiguous sequence of nucleotides within g the sequence set forth in SEQ ID NO:14 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:29 or a sequence having at least or about 85, 90, 91, 92, 93, 94, 95, 96, 97, 98, or 99% sequence identity thereto; (iii) the forward primer comprises a contiguous sequence of nucleotides within a the sequence set forth in SEQ ID NO:15 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:30 or a sequence having at least or about 85, 90, 91, 92, 93, 94, 95, 96, 97, 98, or 99% sequence identity thereto; (iv) the forward primer comprises a contiguous sequence of nucleotides within the sequence set forth in SEQ ID NO:16 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:31 or a sequence having at least or about 85, 90, 91, 92, 93, 94, 95, 96, 97, 98, or 99% sequence identity thereto; (v) the forward primer comprises a contiguous sequence of nucleotides within the sequence set forth in SEQ ID NO:17 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:32 or a sequence having at least or about 85, 90, 91, 92, 93, 94, 95, 96, 97, 98, or 99% sequence identity thereto; (vi) the forward primer comprises a contiguous sequence of nucleotides within the sequence set forth in SEQ ID NO:18 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:33 or a sequence having at least or about 85, 90, 91, 92, 93, 94, 95, 96, 97, 98, or 99% sequence identity thereto; (vii) the forward primer comprises a contiguous sequence of nucleotides within the sequence set forth in SEQ ID NO:19 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:35 or a sequence having at least or about 85, 90, 91, 92, 93, 94, 95, 96, 97, 98, or 99% sequence identity thereto; (viii) the forward primer comprises a contiguous sequence of nucleotides within the sequence set forth in SEQ ID NO:20 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:36 or a sequence having at least or about 85, 90, 91, 92, 93, 94, 95, 96, 97, 98, or 99% sequence identity thereto; (ix) the forward primer comprises a contiguous sequence of nucleotides within the sequence set forth in SEQ ID NO:21 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:38 or a sequence having at least or about 85, 90, 91, 92, 93, 94, 95, 96, 97, 98, or 99% sequence identity thereto; (x) the forward primer comprises a contiguous sequence of nucleotides within the sequence set forth in SEQ ID NO:22 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:39 or a sequence having at least or about 85, 90, 91, 92, 93, 94, 95, 96, 97, 98, or 99% sequence identity thereto; (xi) the forward primer comprises a contiguous sequence of nucleotides within the sequence set forth in SEQ ID NO:23 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:40 or a sequence having at least or about 85, 90, 91, 92, 93, 94, 95, 96, 97, 98, or 99% sequence identity thereto; (xii) the forward primer comprises a contiguous sequence of nucleotides within the sequence set forth in SEQ ID NO:24 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:41 or a sequence having at least or about 85, 90, 91, 92, 93, 94, 95, 96, 97, 98, or 99% sequence identity thereto; (xiii) the forward primer comprises a contiguous sequence of nucleotides within the sequence set forth in SEQ ID NO:25 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:42 or a sequence having at least or about 85, 90, 91, 92, 93, 94, 95, 96, 97, 98, or 99% sequence identity thereto; (xiv) the forward primer comprises a contiguous sequence of nucleotides within the sequence set forth in SEQ ID NO:26 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:43 or a sequence having at least or about 85, 90, 91, 92, 93, 94, 95, 96, 97, 98, or 99% sequence identity thereto; (xv) the forward primer comprises a contiguous sequence of nucleotides within the sequence set forth in SEQ ID NO:27 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:44 or a sequence having at least or about 85, 90, 91, 92, 93, 94, 95, 96, 97, 98, or 99% sequence identity thereto; or (xvi) the forward primer comprises a contiguous sequence of nucleotides within the sequence set forth in SEQ ID NO:28 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:45 or a sequence having at least or about 85, 90, 91, 92, 93, 94, 95, 96, 97, 98, or 99% sequence identity thereto.

In some of any embodiments, the forward and reverse oligonucleotide primer are each independently at least 14 nucleotides in length. In some of any embodiments, the forward and reverse oligonucleotide primer are each independently 14-30 nucleotides in length. In some of any embodiments, the forward and reverse oligonucleotide primer are each independently 18-25 nucleotides in length. In some of any embodiments, the forward and reverse oligonucleotide primer are each independently 18-22 nucleotides in length.

In some of any embodiments, the reverse-transcriptase-dependent amplicon comprises the sequence set forth in SEQ ID NO:10 or a sequence that has at least 80%, at least 85%, at least 90% or at least 95% sequence identity to SEQ ID NO:10. In some of any embodiments, the reverse-transcriptase-dependent amplicon is set forth in SEQ ID NO:10.

In some of any embodiments, the forward oligonucleotide primer comprises a sequence that has at least 80%, at least 85%, at least 90% or at least 95% sequence identity to any one of SEQ ID NOS: 3, 4 or 5. In some of any embodiments, the forward oligonucleotide primer comprises the sequence set forth in any of SEQ ID NOS: 3, 4 or 5. In some of any embodiments, the reverse oligonucleotide primer comprises a sequence that has at least 80%, at least 85%, at least 90% or at least 95% sequence identity to any one of SEQ ID NOS: 6 or 7. In some of any embodiments, the reverse oligonucleotide primer comprises the sequence set forth in any of SEQ ID NOS: 6 or 7. In some of any embodiments, the forward oligonucleotide primer is set forth in SEQ ID NO:3 and the reverse oligonucleotide primer is set forth in SEQ ID NO:6.

In some of any embodiments, the oligonucleotide probe is specific for a third region between the first region and the second region of the reverse-transcribed SIN viral vector DNA. In some of any embodiments, the oligonucleotide probe is specific for a sequence present in an R and/or U5 region of the viral LTR of the reverse-transcribed SIN viral vector DNA. In some of any embodiments, the oligonucleotide probe comprises a sequence that has at least 80%, at least 85%, at least 90% or at least 95% sequence identity to any one of SEQ ID NOS: 8 or 9 or is a complement thereof. In some of any embodiments, the oligonucleotide probe comprises the sequences as set forth in SEQ ID NOs 8 or 9 or is a complement thereof. In some of any embodiments, the oligonucleotide probe comprises the sequence as set forth in SEQ ID NO 8. In some of any embodiments, the oligonucleotide probe comprises the sequence as set forth in SEQ ID NO 9.

In some of any embodiments, the probe further comprises a detectable moiety. In some of any embodiments, the detectable moiety is fluorescent. In some of any embodiments, the detectable moiety is selected from FAM, HEX, FITC, Texas Red, TET, JOE, VIC, NED, TAMRA, ROX, ABY, PET, JUN, LIZ, Cy3, or Cy5.

In some of any embodiments, the one or more regions of reverse-transcribed viral vector DNA are not present in a non-self-inactivating viral vector. In some of any embodiments, the one or more regions of reverse-transcribed viral vector DNA are not present in a wild-type virus or the wild-type from which the viral vector is derived. In some of any embodiments, the one or more regions of reverse-transcribed viral vector DNA is only present after reverse-transcription. In some of any embodiments, the reverse-transcriptase dependent amplicon is not produced from viral vector transduction residuals that are episomal in a cell transduced with the viral vector, optionally wherein said residuals are plasmids.

In some of any embodiments, the sample comprises one or more cells suspected of comprising viral nucleic acid integrated in the genome. In some of any embodiments, the sample comprises one more cells transduced with a viral vector.

In some of any embodiments, the PCR is a multiplex PCR and further comprises producing a reference amplicon of a reference gene in the same reaction. In some of any embodiments, the reference gene is an endogenous gene known to be present in the genome of the cells in the sample. In some of any embodiments, the reference gene is known to be present in one or two copies in a diploid genome. In some of any embodiments, the reference gene is selected from hTert, β-actin, GAPDH, ribonuclease P/MRP subunit 30 (RPP30).

In some of any embodiments, the viral vector copy number is the copy number or the average copy number of the generated reverse-transcriptase-dependent amplicon per cell in the sample. In some of any embodiments, the viral vector copy number is the copy number or the average copy number of the generated reverse-transcriptase-dependent amplicon per diploid genome. In some of any embodiments, the vector copy number per diploid genome is calculated as the ratio between the number of copies of the generated reverse-transcriptase-dependent amplicon and the reference amplicon. In some embodiments, the ratio is multiplied by two, e.g., where the reference gene is present on an autosome or the reference gene is present on an X chromosome and the sample is from a female subject. In some embodiments, the ratio is multiplied by one, e.g., where the reference gene is present on an X chromosome and the sample is from a male subject.

Provided herein is a composition comprising (i) a forward oligonucleotide primer that comprises a contiguous sequence of nucleotides of the plus strand of a first region present within the deleted U3 (delU3) of a reverse-transcribed self-inactivating (SIN) viral vector DNA, and (ii) a reverse oligonucleotide primer that comprises a contiguous sequence of nucleotides of the minus strand of a second region downstream of the first region in the reverse-transcribed viral vector DNA to make a replicable reverse-transcriptase-dependent amplicon.

In some of any embodiments, the reverse-transcriptase-dependent amplicon is 100 base pairs to 500 base pairs in size. In some of any embodiments, the reverse-transcriptase-dependent amplicon is 200 base pairs to 400 base pairs in size.

In some of any embodiments, the first region comprises the U3 attachment sequence. In some of any embodiments, the first region comprises the sequence set forth in any one of SEQ ID NOS: 46-48. In some of any embodiments, the forward primer comprises at least a contiguous portion of any one of SEQ ID NOS: 46-48. In some of any embodiments, the first region comprises the sequence set forth in any one of SEQ ID NOS: 1 and 14-28 or a sequence having at least or about 85, 90, 91, 92, 93, 94, 95, 96, 97, 98, or 99% sequence identity to any one of SEQ ID NOS: 1 and 14-28. In some of any embodiments, the first region is set forth in SEQ ID NO:1.

In some of any embodiments, the second region comprises at least a portion of the primer binding site (PBS), the IFN-stimulated response element (ISRE), and/or the Psi (Ψ) packaging sequence of the SIN viral vector. In some of any embodiments, the second region comprises the sequence set forth in any one of SEQ ID NOS: 2 and 29-45 or a sequence having at least or about 85, 90, 91, 92, 93, 94, 95, 96, 97, 98, or 99% sequence identity to any one of SEQ ID NOS: 2 and 29-45.

In some of any embodiments, (i) the forward primer comprises a contiguous sequence of nucleotides within the sequence set forth in SEQ ID NO:1 or a sequence having at least or about 85, 90, 91, 92, 93, 94, 95, 96, 97, 98, or 99% sequence identity thereto, and the reverse primer comprises a contiguous sequence of nucleotides within the sequence set forth in SEQ ID NO:2 or a sequence having at least or about 85, 90, 91, 92, 93, 94, 95, 96, 97, 98, or 99% sequence identity thereto; (ii) the forward primer comprises a contiguous sequence of nucleotides within the sequence set forth in SEQ ID NO:14 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:29 or a sequence having at least or about 85, 90, 91, 92, 93, 94, 95, 96, 97, 98, or 99% sequence identity thereto; (iii) the forward primer comprises a contiguous sequence of nucleotides within the sequence set forth in SEQ ID NO:15 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:30 or a sequence having at least or about 85, 90, 91, 92, 93, 94, 95, 96, 97, 98, or 99% sequence identity thereto; (iv) the forward primer comprises a contiguous sequence of nucleotides within the sequence set forth in SEQ ID NO:16 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:31 or a sequence having at least or about 85, 90, 91, 92, 93, 94, 95, 96, 97, 98, or 99% sequence identity thereto; (v) the forward primer comprises a contiguous sequence of nucleotides within the sequence set forth in SEQ ID NO:17 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:32 or a sequence having at least or about 85, 90, 91, 92, 93, 94, 95, 96, 97, 98, or 99% sequence identity thereto; (vi) the forward primer comprises a contiguous sequence of nucleotides within the sequence set forth in SEQ ID NO:18 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:33 or a sequence having at least or about 85, 90, 91, 92, 93, 94, 95, 96, 97, 98, or 99% sequence identity thereto; (vii) the forward primer comprises a contiguous sequence of nucleotides within the sequence set forth in SEQ ID NO:19 or a sequence having at least or about 85, 90, 91, 92, 93, 94, 95, 96, 97, 98, or 99% sequence identity thereto and the reverse primer comprises a contiguous sequence of nucleotides within a the sequence set forth in SEQ ID NO:35 or a sequence having at least or about 85, 90, 91, 92, 93, 94, 95, 96, 97, 98, or 99% sequence identity thereto; (viii) the forward primer comprises a contiguous sequence of nucleotides within the sequence set forth in SEQ ID NO:20 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:36 or a sequence having at least or about 85, 90, 91, 92, 93, 94, 95, 96, 97, 98, or 99% sequence identity thereto; (ix) the forward primer comprises a contiguous sequence of nucleotides within the sequence set forth in SEQ ID NO:21 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:38 or a sequence having at least or about 85, 90, 91, 92, 93, 94, 95, 96, 97, 98, or 99% sequence identity thereto; (x) the forward primer comprises a contiguous sequence of nucleotides within the sequence set forth in SEQ ID NO:22 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:39 or a sequence having at least or about 85, 90, 91, 92, 93, 94, 95, 96, 97, 98, or 99% sequence identity thereto; (xi) the forward primer comprises a contiguous sequence of nucleotides within the sequence set forth in SEQ ID NO:23 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:40 or a sequence having at least or about 85, 90, 91, 92, 93, 94, 95, 96, 97, 98, or 99% sequence identity thereto; (xii) the forward primer comprises a contiguous sequence of nucleotides within the sequence set forth in SEQ ID NO:24 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:41 or a sequence having at least or about 85, 90, 91, 92, 93, 94, 95, 96, 97, 98, or 99% sequence identity thereto; (xiii) the forward primer comprises a contiguous sequence of nucleotides within the sequence set forth in SEQ ID NO:25 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:42 or a sequence having at least or about 85, 90, 91, 92, 93, 94, 95, 96, 97, 98, or 99% sequence identity thereto; (xiv) the forward primer comprises a contiguous sequence of nucleotides within the sequence set forth in SEQ ID NO:26 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:43 or a sequence having at least or about 85, 90, 91, 92, 93, 94, 95, 96, 97, 98, or 99% sequence identity thereto; (xv) the forward primer comprises a contiguous sequence of nucleotides within the sequence set forth in SEQ ID NO:27 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:44 or a sequence having at least or about 85, 90, 91, 92, 93, 94, 95, 96, 97, 98, or 99% sequence identity thereto; or (xvi) the forward primer comprises a contiguous sequence of nucleotides within the sequence set forth in SEQ ID NO:28 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:45 or a sequence having at least or about 85, 90, 91, 92, 93, 94, 95, 96, 97, 98, or 99% sequence identity thereto.

In some of any embodiments, the forward and reverse oligonucleotide primer are each independently at least 15 nucleotides in length. In some of any embodiments, the forward and reverse oligonucleotide primer are each independently 15-30 nucleotides in length. In some of any embodiments, the forward and reverse oligonucleotide primer are each independently 18-25 nucleotides in length. In some of any embodiments, the forward and reverse oligonucleotide primer are each independently 18-22 nucleotides in length.

In some of any embodiments, the reverse-transcriptase-dependent amplicon comprises the sequence set forth in SEQ ID NO:10 or a sequence that has at least 80%, at least 85%, at least 90% or at least 95% sequence identity to SEQ ID NO:10. In some of any embodiments, the reverse-transcriptase-dependent amplicon is set forth in SEQ ID NO:10.

In some of any embodiments, the forward oligonucleotide primer comprises a sequence that has at least 80%, at least 85%, at least 90% or at least 95% sequence identity to any one of SEQ ID NOS: 3, 4 or 5. In some of any embodiments, the forward oligonucleotide primer comprises the sequence set forth in any of SEQ ID NOS: 3, 4 or 5. In some of any embodiments, the reverse oligonucleotide primer comprises a sequence that has at least 80%, at least 85%, at least 90% or at least 95% sequence identity to any one of SEQ ID NOS: 6 or 7. In some of any embodiments, the reverse oligonucleotide primer comprises the sequence set forth in any of SEQ ID NOS: 6 or 7. In some of any embodiments, the forward oligonucleotide primer is set forth in SEQ ID NO:3 and the reverse oligonucleotide primer is set forth in SEQ ID NO:6.

In some of any embodiments, the composition further comprises a polymerase enzyme. In some of any embodiments, the polymerase enzyme is a DNA polymerase, optionally a Taq DNA polymerase, Hot Start DNA polymerase and/or a high-fidelity DNA polymerase.

In some of any embodiments, the composition further comprises an oligonucleotide probe. In some of any embodiments, the oligonucleotide probe is specific for a sequence present in an R and/or U5 region of the viral LTR. In some of any embodiments, the oligonucleotide probe comprises a sequence that has at least 80%, at least 85%, at least 90% or at least 95% sequence identity to any one of SEQ ID NOS: 8 or 9 or is a complement thereof.

In some of any embodiments, the probe comprises the nucleic acid sequence set forth in any of SEQ ID NOs 8 or 9. In some of any embodiments the probe further comprises a detectable moiety, optionally wherein said moiety is fluorescent. In some of any embodiments, the detectable moiety is selected from FAM, HEX, FITC, Texas Red, TET, VIC, TAMRA, ROX, LIZ, Cy3, or Cy5.

Also provided herein is a probe, comprising a nucleic acid sequence set forth in any of SEQ ID NOs. 8 or 9. In some of any embodiments, the probe further comprises a detectable moiety. In some of any embodiments, the detectable moiety is fluorescent. In some of any embodiments, the detectable moiety is selected from FAM, HEX, FITC, Texas Red, TET, VIC, TAMRA, ROX, LIZ, Cy3, or Cy5.

Provided herein is a kit comprising any of the provided compositions and/or any of the provided probes. Also provided herein is a kit comprising (i) a forward oligonucleotide primer comprising a nucleic acid sequence set forth in any of SEQ ID NOs 3-5, (ii) a reverse oligonucleotide primer comprising a nucleic acid sequence set forth in any of SEQ ID NOs 6-7, and (iii) a probe comprising a nucleic acid sequence set forth in any of SEQ ID NOs 8 or 9. In some of any embodiments, (i) the forward oligonucleotide primer comprises a nucleic acid sequence set forth in SEQ ID NO. 3, (ii) the reverse oligonucleotide primer comprises a nucleic acid sequence set forth in SEQ ID NO. 7, and (iii) the probe comprises a nucleic acid sequence set forth in SEQ ID NO. 8.

Provided herein is a reaction mixture, comprising any of the provided compositions. In some of any embodiments, the mixture further comprises a sample. In some of any embodiments, the sample comprises DNA from one or more cells transduced with a self-inactivating (SIN) viral vector. In some of any embodiments, the sample comprises DNA from one or more cells suspecting of comprising SIN viral nucleic acid integrated in the genome. In some of any embodiments, the reaction mixture further comprises all four deoxyribonucleoside triphosphates, and/or an appropriate buffer, optionally wherein said buffer comprises Mn+2.

### BRIEF DESCRIPTION OF THE DRAWINGS

FIG. 1 depicts the process of Human Immunodeficiency Virus (HIV) reverse transcription. Exemplary primers according to embodiments provided herein are noted with arrows, while the probe for detecting a region of reverse-transcribed DNA corresponding to a reverse transcription-dependent amplicon (designated “DelU3” herein) is noted with a star (Image adapted from Coffin et al. “Retroviruses” Cold Spring Harbor Laboratory Press; 1997).

FIG. 2A depicts an exemplary amplitude plot readout of a DelU3 reverse-transcriptase dependent amplicon (FAM signal) and reference gene (hTert) amplicon (HEX signal) from a digital droplet PCR (ddPCR) run. FIG. 2B depicts a thermogradient reflecting amplitude differences of the DelU3 reverse-transcriptase dependent amplicon following amplification by different exemplary primer pairs. FIG. 2C depicts exemplary results for detection of the DelU3 reverse-transcriptase dependent amplicon using an exemplary primer pair across a vector dosage range.

FIG. 3A. depicts detection of viral copy number (VCN) using traditional WPRE amplification in the presence or absence of nevarapine (Nev) treatment.

FIG. 3B depicts the analysis of GFP expression as a result of successful expression of the GFP reporter transgene present on an exemplary integrated vector. Determination of VCN based on assay of the DelU3 amplicon with Nevirapine treatment is shown in FIG. 3C

FIG. 3D depicts detection of VCN by amplification of a DelU3 reverse-transcriptase dependent amplicon in accord with embodiments herein in the presence or absence of nevarapine treatment. FIG. 3E shows the results of nevarapine normalization performed to confirm vector copy integration into the genome using traditional WPRE amplification. FIG. 3F depicts comparison of a series of exemplary assays in which VCN was calculated by detecting the WPRE amplicon followed by benzonase treatment or using the methods as described herein by amplification of a DelU3 reverse-transcriptase dependent amplicon. FIG. 3G depicts similar results in a series of assays in which the timing of benzonase treatment was varied.

FIG. 4A (resting PBMCs) and FIG. 4B (activated PBMCs) depict determination of vector copy number (VCN) after transduction with different pseudotyped lentiviral vectors.

FIG. 5A (Active Pan T) and FIG. 5B (resting Pan T) depict determination of vector copy number (VCN) after transduction with different pseudotyped lentiviral vectors. FIG. 5C shows the VCN of isolated murine hepatocytes

FIG. 6 depicts comparison of a series of exemplary assays in which VCN was calculated by detecting the WPRE amplicon followed by Nev normalization (WPRE Nev normalization) or using the methods as described herein by amplification of a DelU3 reverse-transcriptase dependent amplicon.

### DETAILED DESCRIPTION

Provided herein are methods involving amplification of a reverse-transcriptase dependent amplicon if present in a sample that has been transduced with a self-inactivating (SIN) viral vector. In some embodiments, the SIN viral vector is a retroviral vector such as a lentiviral vector (LV). In some embodiments, the SIN viral vector is a retroviral vector such as a gamma-retroviral vector. In some embodiments, the SIN viral vector is a gamma-retroviral vector such as a Moloney Murine Leukemia Virus (MLV) vector. The provide methods include nucleic acid amplification (e.g., PCR) methods using primers specific for a region of reverse-transcribed viral DNA in the presence of a DNA polymerase. In some embodiments, the provided methods of amplification include a forward primer and a reverse primer that are each independently complementary to a different region of reverse transcribed SIN viral vector (e.g. LV) DNA. In some embodiments, the forward primer is a first region that is a contiguous sequence of nucleotides within or overlapping the deleted U3 (delU3) region of reverse transcribed SIN viral vector DNA (i.e., plus strand) and the reverse primer is a second region that is a contiguous sequence of nucleotides (i.e., minus strand) downstream of the first region of reverse-transcribed SIN viral vector DNA to make a replicable amplicon. In some embodiments, the second region is downstream of the U5 of the reverse-transcribed SIN viral vector DNA and includes sequences of the primer binding site (PBS) and/or Psi. In some embodiments, the reverse-transcriptase dependent amplicon that is amplified by the present method is or contains nucleic acids from the delU3 if present in the sample (also referred to as the “delU3 amplicon”). Also provided are methods that further include detecting the generated reverse-transcriptase-dependent amplicon.

In some embodiments, DNA from a sample known or suspected of containing SIN viral vector (e.g. LV) DNA is incubated with the forward and reverse primers together with a DNA polymerase in order to mediate amplification of the reverse-transcriptase amplicon, if present, by nucleic acid amplification (e.g., polymerase chain reaction (PCR)). In some embodiments, the PCR is a quantitative PCR, such as real-time (RT)-PCR. In some embodiments, the incubating with the forward and reverse primers further includes an oligonucleotide probe specific for a third region, or a complement thereof, of the reverse-transcribed viral DNA. In particular embodiments, the third region is a region that is between the first region and the second region of the reverse-transcriptase amplicon, on either DNA strand. In some embodiments, the third region is a contiguous sequence of nucleotides within the U5 or R region of the LTR. In some embodiments, the oligonucleotide probe further includes a detectable moiety. In some aspects, the methods may include quantitative PCR by detecting a signal from the detectable moiety, and quantifying an amount of the generated reverse-transcriptase-dependent amplicon by detection of the detectable signal from the detectable moiety.

Also provided herein are methods of determining vector copy number (VCN) of a SIN viral vector (e.g. LV, MLV, gamma-retroviral, etc.) that has been transduced into a cell. The methods may include methods that involve amplifying a reverse-transcriptase dependent amplicon and quantifying an amount of the reverse-transcriptase dependent amplicon in accord with any of the provided method, and, based on the amount of the reverse-transcriptase dependent amplicon, determining viral vector copy number in the sample. In some embodiments, the amplifying and quantifying is carried out by quantitative PCR, such as real-time PCR. In some embodiments, the viral vector copy number is the copy number or the average copy number of the reverse-transcriptase-dependent amplicon per cell in the sample. In some embodiments, viral vector copy number may be determined in a multiplex PCR assay by comparison of the amount of the reverse-transcriptase amplicon to a reference amplicon (e.g., an amplicon from a housekeeping gene or other reference gene that is known to be present in one or two copies in a diploid genome). In some embodiments, the viral vector copy number is the copy number or the average copy number of the generated reverse-transcriptase-dependent amplicon per diploid genome. In some embodiments, the vector copy number per diploid genome is calculated as the ratio between the number of copies of the generated reverse-transcriptase-dependent amplicon and the reference amplicon. In some embodiments, the vector copy number per diploid genome is calculated as the ratio between the number of copies of the generated reverse-transcriptase-dependent amplicon and the reference amplicon and further multiplied by two.

The provided embodiments are based on design of an improved method for detecting amplified products from SIN viral vectors, such as LVs and other retroviral vectors, particularly in connection with VCN determination. VCN determination is an important readout for any viral-based method involving a SIN viral vector, including lentiviral (LV) gene therapy. SIN viral vectors, such as lentiviral and gamma-retroviral vectors, are advantageous because they transfer genetic material so that it is integrated into the genome of a cell without subsequent transcription of the complete viral genome. Lentiviral vector (LV) and gamma-retroviral vector production residuals may be present within any viral production run. These may include, for example, one or more of vector plasmids, linear complementary DNA (cDNA), autointegrants or long terminal repeat (LTR) circles. Existing methods, however, do not accurately or consistently distinguish between integrated nucleic acid derived from a SIN viral vector and other residual plasmids derived from a SIN viral vector that may be present in a transduced cell sample, thereby resulting in an overestimation of integrated viral vector. For instance, standard production of lentiviral vectors may contain certain plasmid residual contaminants which may remain even after benzoase treatment. Due to the presence of the residual plasmids, many existing methods are not able to measure VCN that is transduction-specific versus due to residual plasmid DNA present in a lentiviral vector preparation. This is particularly a problem for existing qPCR-based methods because the qPCR amplicon is present in both the integrated vector DNA and residual plasmid. As an example, methods that utilize a WPRE amplicon, which is not specific to reverse-transcriptase dependent SIN viral vector nucleic acids, will pick up other plasmid residuals.

Whereas other downstream steps can be carried out after benzonase treatment to ensure removal of all residual plasmid DNA and other residuals, these methods require a number of additional steps which are time-consuming and may impact sample availability. These extra steps may also impact accuracy, precision and/or reproducibility of the method. For instance, other downstream methods to remove plasmid residuals prior to VCN determination include, but are not limited to, ultracentrifugation through a sucrose cushion (research-grade vector), column chromatography (GMP-grade vector) and/or treatment with nevirapine. Nevirapine is a reverse-transcriptase inhibitor that blocks RNA vector genome conversion into vector DNA, which is the target for qPCR. Methods using nevirapine thus require two assays involving the presence of nevirapine (specific for only amounts of plasmid residual DNA) and absence of nevirapine (specific for all DNA, including integrated viral DNA and plasmid residual DNA) for determination of transduction-specific DNA.

Thus, to date production residuals have made accurate and reproducible detection of transduction-specific VCN difficult due to contaminating plasmid residuals that can still be present after benzonase treatment. Having an assay that is only specific for reverse-transcriptase dependent SIN (e.g. LV and gamma-retroviral) viral vector nucleic acid products allows for more accurate determination of vector copy number without having the need for extensive downstream processing. Importantly, this method may replace existing assay methods that often utilize a WPRE amplicon, which is not specific to reverse-transcriptase dependent viral vector nucleic acids and will pick up other plasmid residuals.

The present methods are based on exploiting a new amplicon that is specific to only reverse-transcribed self-inactivating (SIN) viral vector nucleic acids. The SIN viral vector nucleic acids may include both integrated (as in the case of integration-competent LV and gamma-retroviral vectors) and unintegrated (in the case of integration-deficient LV and gamma-retroviral vectors) nucleic acids. The provided methods allow for accurate determination of SIN VCN in the presence of production residuals such as plasmid. The detection of only reverse-transcribed nucleic acid SIN viral vector (e.g. LV and gamma-retroviral) products relies on the specific orientation of primers and probes to only form an amplicon after reverse transcription has been completed. This amplicon can then be detected by PCR and quantified using qPCR-based methods, such as RT-PCR or ddPCR. This method can be applied to any SIN retroviral vector design, including HIV, SIV, and gammaretroviral vectors such as MLV.

In some embodiments, the SIN viral vector is a retroviral vector, such as a lentiviral vector. In some embodiments, the SIN viral vector is a gamma-retroviral vector, such as a MLV vector.

In some embodiments, the viral vector has a long terminal repeat sequence (LTR). In the native provirus, viral genes are flanked by the LTRs. The structure of a wild-type retrovirus genome can comprise a 5′ (LTR) and a 3′ LTR. The LTRs are involved in proviral integration and transcription. In some aspects, the LTRs can also serve as enhancer-promoter sequences and can therefore in some aspects control the expression of the viral genes. The LTR can comprise numerous regulatory signals including transcriptional control elements, polyadenylation signals and sequences for replication and integration of the viral genome.

The viral LTR is typically divided into three regions called U3, R and U5. U3 is derived from the sequence unique to the 3′ end of the RNA, while R is derived from a sequence repeated at both ends of the RNA. U5 is derived from the sequence unique to the 5′ end of the RNA. The sizes of these three regions can vary among different retroviruses. The U3 region typically contains the enhancer and promoter elements. For instance, the U3 region of a wildtype reference HIV-1 lentivirus is 454 bp in length and can also be further divided into three domains based on transcription factor binding: the modulatory, enhancer, and core promoter domains. In the case of gamma-retroviral MLV, the U3 region is 447 bp in length and similarly divided into modulatory, enhancer, and core domains. The U5 region is typically the sequence between the primer binding site and the R region and can contain the polyadenylation sequence. The R (repeat) region can be flanked by the U3 and U5 regions. In some embodiments, adjacent to the 5′ LTR are sequences for reverse transcription of the genome (the tRNA primer binding site) and for efficient packaging of viral RNA into particles (e.g. the Psi site). In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi sequence) for encapsidation of the viral genome. For instance, encapsidation of the retroviral RNAs occurs by virtue of a psi sequence located at the 5′ end of the viral genome.

Retroviral genomic RNA is plus-stranded, whereby mRNA for protein expression and further copies of genomic RNA are produced from the same strand. In some aspects, the synthesis primer for the first DNA stand (i.e., the minus strand) is a host tRNA. Also present is a primer binding site (PBS) to which the host tRNA binds, and at least two integration sites to enable integration into a host cell genome. The associated gag, pol and env genes encode the packaging components which promote the assembly of the viral vector particles (or viral particles).

The site of transcription initiation for the viral genome is typically at the boundary between U3 and R in one LTR. The site of poly (A) addition (i.e., transcriptional termination) is at the boundary between R and U5 in the other LTR. U3 contains most of the transcriptional control elements of the provirus, which include the promoter and multiple enhancer sequences responsive to cellular and in some cases, viral transcriptional activator proteins. The promoter and enhancer domains of the U3 are transferred to the 5′ LTR of progeny proviral DNA as a result of reverse-transcription and function to regulate transcription. In addition to promoters and enhancers, the U3att sequence also located within the U3 modulatory region is required for efficient interaction with IN (integrase).

In some embodiments, a SIN viral vector, such as a SIN lentiviral vector or SIN gamma-retroviral vector (i.e., MLV, SIV or HIV-1), has LTR regions which do not permit replication. In some embodiments, in a SIN retroviral vector, both LTR sequences may be modified to generate the self-inactivating vector. For instance, a SIN vector typically includes a deleted U3 (delU3) in which a large part of the U3 region is deleted, including portions containing the transcriptional enhancer and promoter. By deleting the transcriptional enhancers and/or the promoter in the U3 region of the LTR, the vector is replication limited so that following reverse transcription a full-length LTR cannot be reconstituted. In some aspects, SIN vectors have a deletion in the 3′-LTR covering the promoter/enhancer elements from the U3 region, e.g. about a 50 to about a 400 base pair deletion.

In some embodiments, a SIN viral vector contains a delU3 region that preferably includes the att sequence (e.g. set forth in any one of SEQ ID NOS: 46-48), but lacks any sequences having promoter activity, thereby causing the vector to be self-inactivating (SIN) in that viral transcription cannot go beyond the first round of replication following chromosomal integration. In some embodiments, a SIN viral vector contains a delU3 in which a U3att sequence set forth in any one of SEQ ID NOS: 46-48 is retained. In some embodiments, a SIN viral vector contains a delU3 in which only the minimal U3 att sequence is retained. In some embodiments, a SIN viral vector contains a delU3 in which the U3 att sequence and most of the U3 modulatory region is retained with deletions of the enhancer and core promoter U3 regions. In some embodiments, the SIN vector comprises a deleted U3 region, wherein said deletion includes a deletion of the TATA box. The deletion may be one that removes the TATA box, preventing transcription initiation and therefore inactivating the virus Miyoshi et al. 1998; Zuffrey et al 1998). In some aspects, this 3′-LTR deletion removes the polyadenylation signal distal to the TATA box. In some aspects, the 3′-LTR deletion removes the integrase recognition and processing site.

In some aspects, the SIN vector comprises a deletion of the U3 enhancer and/or core regions. In some aspects, the SIN vector comprises a deletion of at least 400, at least 350, at least 300, at least 250, at least 200, at a least 150, at least 100, or at least 50 base pairs within the U3, wherein said deletion includes a deletion of the TATA box. In some aspects, the SIN vector comprises a deletion of 150 base pairs within the U3 core promoter domain, wherein said deletion includes a deletion of the TATA box. In some aspects, the SIN vector comprises a deletion of 134 base pairs within the U3 core promoter domain, wherein said deletion includes a deletion of the TATA box. In some embodiments, the SIN vector comprises a deleted U3 in which TCF-1α and TATA sequences are deleted.

In some aspects, the U3 region of the 5′-LTR of the SIN viral vector has been replaced by other heterologous promoting sequences (e.g. CMV or RSV) to achieve a Tat-independent transcription and to increase genomic RNA synthesis, resulting in an increase in viral titer by SIN vectors. Because 5′-U3 region drives the expression of primary transcripts, its modifications will not be present in transduced cells (Schambach et al. 2009). In some aspects, the R region of the viral 3′ LTR of SIN vectors also include exogenous elements, such as 0-globin or SV40 polyadenylation signals or the upstream sequence element (USE) from simian virus 40 (SV40-USE) in order to decrease the transcriptional readthrough from the internal promoters or from remnants of the deleted U3 region of SIN-LV vectors (Almarza et al. 2011) preventing the potential transcriptional activation of the downstream genes. The skilled artisan is readily familiar with sequences of the modified 3′ and 5′ LTRs of SIN retroviral vectors.

In a replication-defective or self-inactivating retroviral vector genome gag, pol and env may be entirely absent or otherwise functional.

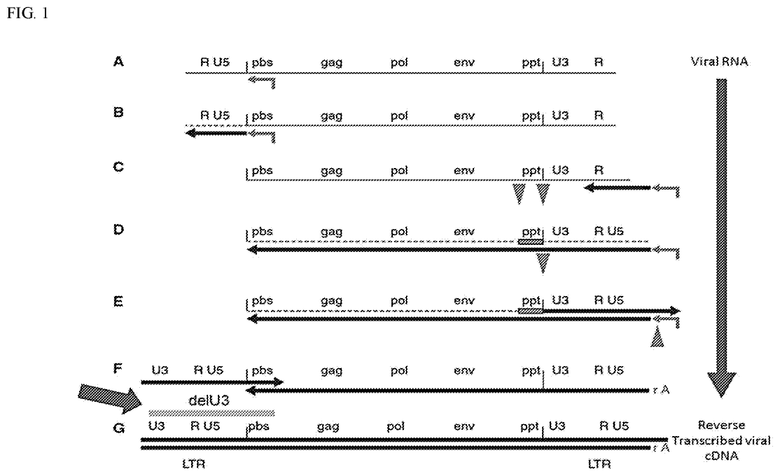

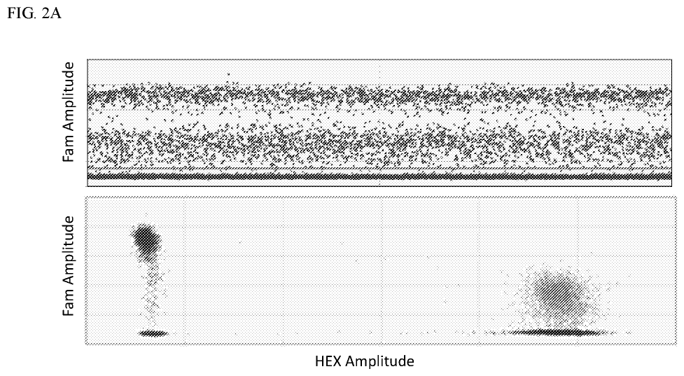

As shown in FIG. 1, following a single round of reverse transcription of the SIN nucleic acids, these alterations to the 3′ LTR (U3) are copied into both the 3′ and 5′ ends resulting in a transcriptionally inactive provirus. Specifically, during assembly, cellular tRNA, which is complementary to the PBS of the plus strand of the reverse-transcribed viral vector DNA, is used as a primer for the initiation of the reverse transcription. This results in the initial priming where single negative-stranded DNA is transferred to the 3′ end and annealed via R region so that the U5 nucleotide sequence primes the 3′ end of the viral genome. This initiates DNA synthesis of the entire minus DNA strand followed by priming for synthesis of the positive DNA strand from the polypurine tract (PPT). During reverse transcription, the R domain in the LTR is involved in strand transfer and recreates the 5′ U3 from the 3′ LTR. This mechanism allows for alternative promoters for the vector, such as any described in Section III, to remain intact as internal to the LTR sequences. Deletions in the 3′ LTR are incorporated into the 5′ LTR during reverse-transcription, and therefore an integrated provirus (i.e., an integrated vector sequence) lacks any of the enhancers necessary to regulate transcription.

The provided methods relate to aspects of SIN viral vectors that result following reverse transcription, such as may occur after integration of a viral vector into the genome of a cell. Transferring the delU3 to the 5′ end generates a unique amplicon formed by amplification of regions of the reverse-transcribed nucleic acid containing the delU3. In the provided methods, primers are designed to detect the delU3 unique amplicon that is specific to reverse-transcribed SIN viral vector nucleic acids as a result of the transfer of the delU3 to the 5′ end as occurs during reverse transcription (FIG. 1).

Exemplary SIN vectors are known in the art. In some aspects, the methods can be carried out on DNA from any cell that is known or suspected of having been transduced with a SIN viral vector. Exemplary SIN viral vectors include any containing a deletion or truncation of the 3′-LTR, such as any as described. In some aspects, the SIN vector comprises a deleted U3 region or delU3. A non-limiting list of SIN vectors known in the art is set forth below in Table 1 (see also Johnson et al., 2021, Mol. Ther: Methods & Clinical Development, 21:451-465).

TABLE 1
Exemplary SIN Vectors and Backbones

| | |
|---|---|
| Clinical Trial or Cat No | Reference |

Lentivirus

| | | |
|---|---|---|
| pHIV-7 | NCT01153646 | Johnson et al (2021). HIV- |
| | | based lentiviral vectors: Origin |
| | | and sequence differences. Mol |
| | | Therp: Methods & Clinical |
| | | Development Vol 21: 451-465 |
| pCCLsin | NCT01560182, | |
| | NCT01560182, | |
| | NCT01515462, | |
| | NCT00991224, | |
| | NCT02247843, | |
| | NCT01381003 - 2012, | |
| | NCT01545323, | |
| | NCT02247843, | |
| | NCT02234934, | |
| | NCT01380990, | |
| | NCT02757911 - 2016, | |
| | NCT02453477, | |
| | NCT03488394, NCT01560182 | |
| pHR-SIN-TRIP | Addgene; cat #12265 | |
| pTNS9 | NCT01639690 | |
| pWPXL | NCT01380990 - 2011 | |
| pRRLsin | NCT01515462, | |
| | NCT01029366 - 2009 | |
| pNL ′U3 | PMID: 10849549 | |
| pTYF | NCT03645486, | |
| | NCT03727555, | |
| | NCT03645460, NCT03217617 | |
| pCSCIGW | | Ken Cornetta (NGTB), |
| | | National Gene Vector |
| | | Biorepository |
| pLenti-III | Applied Biological Materials; | Wang et al. (2018). iNOS |
| | cat # LV587 | promotes CD24+CD133+ liver |
| | | cancer stem cell phenotype |
| | | through a TACE/ADAM17- |
| | | dependent Notch signaling |
| | | pathway. Proc. Natl. Acad. Sci. |
| | | USA 115, E10127-E10136. |
| pRSIEG | Cellecta; SVSHU6EG-L | |
| pHage | Addgene; cat #46793 | Wilson et al. (2010). |
| | | Amelioration of emphysema |
| | | in mice through lentiviral |
| | | transduction of long-lived |
| | | pulmonary alveolar |
| | | macrophages. |
| | | J. Clin. Invest. 120, 379-389. |
| pSF Lenti | Sigma; #OGS269 | |
| pLenti7.3 | Thermo Fisher Scientific; cat | |
| | #V53406 | |
| P156RRLsinppt | Addgene; cat #42795 | Zhu et al. (2011). BRCA1 |
| | | tumour suppression occurs via |
| | | heterochromatin-mediated |
| | | silencing. Nature 477, 179- |
| | | 184. |
| pLL3.7m | Addgene; cat #89362 | Zhou et al (2017). Optical |
| | | control of cell |
| | | signaling by single-chain |
| | | photoswitchable kinases. |
| | | Science 355, 836-842. |
| pLV-GFPSpark | Sinobiological; cat #LVCV-01 | |
| pLVX | Takara; cat #632164); | |

Gamma-Retrovirus

| | | |
|---|---|---|
| pMSCV mSin1.1 | Addgene; cat #12582 | |
| pMSCVsin-PGK | Addgene; cat # 35615 | |
| pSIR-neo | Addgene; cat # 51128 | Fujita et al. (2014). |
| | | Identification of Proteins |
| | | Associated with an |
| | | IFNgamma-Responsive |
| | | Promoter by a Retroviral |
| | | Expression System for enChIP |
| | | Using CRISPR. PLoS One 7. |
| ID | | Kim et al. (2010), |
| | | Development of murine |
| | | leukemia virus-based retroviral |
| | | vectors with a minimum |
| | | possibility of cis-activation. |
| | | Gene therapy 18, 240-249. |
| plU3 | | Fomin et al. (2014). The U3 |
| | | Region of Moloney Murine |
| | | Leukemia Virus Contains |
| | | Position-independent Cis- |
| | | acting Sequences Involved in |
| | | the Nuclear Export of Full- |
| | | length Viral Transcripts. JBC |
| | | 18, 20158-20169. |
| SIN-MLV.K.B3 | | Mavilio et al. (2013). Self- |
| | | inactivating MLV vectors have |
| | | a reduced genotoxic profile in |
| | | human epidermal |
| | | keratinocytes. Gene therapy 20, |
| | | 949-957. |
| SIN.CMV; SIN.CMV.W.; | | Caruso et al. (2019). |
| SIN.CMV.W.SV | | Generation of High-Titer Self- |
| | | Inactivated gamma-Retroviral |
| | | Vector Producer Cells. |
| | | Molecular therapy 14, 90-99. |
| SIN.RSV | | Caruso et al. (2019). |
| | | Generation of High-Titer Self- |
| | | Inactivated gamma-Retroviral |
| | | Vector Producer Cells. |
| | | Molecular therapy 14, 90-99. |
| SIN.CAG | | Caruso et al. (2019). |
| | | Generation of High-Titer Self- |
| | | Inactivated gamma-Retroviral |
| | | Vector Producer Cells. |
| | | Molecular therapy 14, 90-99. |

In some embodiments, the provided methods utilize droplet digital PCR (ddPCR) which is a PCR-based method that allows precise quantification and analysis of DNA. In some embodiments, the ddPCR methods employ two detectable oligonucleotide probes, one that binds to an amplified reference gene (e.g. housekeeping gene) expected to be present in all samples in the assay (e.g. with a specific forward and reverse primer) and a reverse-transcription dependent vector probe that binds to a sequence of the amplicon as described in accord with the above methods as an indicator of vector copy number. In some embodiments, a detectable signal (e.g. fluorescent signal) for the two probes can be determined, and the ratio of droplets positive for both the reference and reverse-transcription dependent vector probe versus only the reference probe indicates the amount of vector copies present in the sample. In some embodiments, an average number of copies present in the cell population from which the sample was derived also can be determined.

The provided methods thus amplify reverse-transcribed SIN viral nucleic acids and are not able to amplify plasmid residuals. In some embodiments, this ensures a reduction in false-positives and a reduction in over-estimation of non-reverse transcribed SIN viral nucleic acids. In some embodiments, the provided methods result in a high or superior accuracy, precision or reproducibility of the method for detecting or determining reverse-transcribed SIN viral nucleic acids, or determining vector copy number therefrom, in transduced cells, particularly compared to other qPCR methods based on an amplicon also present in a residual plasmid (e.g. WPRE). In some embodiments, the accuracy, precision and/or reproducibility may be tested by carrying out replicates of the assay. In some embodiments, a single assay is conducted by performing the assay on a particular sample in duplicate or triplicate. In some embodiments, the assay is performed in duplicate. In some embodiments, the assay is performed in triplicate. In some embodiments, the replicates of the assay are carried out by different users to assess the reproducibility or repeatability of the results. It is within the level of a skilled artisan to determine or assess the accuracy, precision or reproducibility of the provided methods.

In some embodiments, the provided methods result in an accuracy of the method for detection or determination of reverse-transcribed SIN viral nucleic acids, or determination of vector copy number therefrom, in transduced cells that is greater than at or about 90%, such as greater than at or about 95%, greater than at or about 96%, greater than about or about 97%, greater than at or about 98%, greater than at or about 99%, or at or about 100%. In some embodiments, the provided methods result in improved accuracy for detection or determination of reverse-transcribed SIN viral nucleic acids, or determination of vector copy number therefrom, in transduced cells, in particular compared to a standard assay involving a qPCR based method for an amplicon also present on a plasmid residual (e.g. WPRE).

In some embodiments, the provided methods result in a precision of the method for detection or determination of reverse-transcribed SIN viral nucleic acids, or determination of vector copy number therefrom, in transduced cells that is greater than at or about 90%, such as greater than at or about 95%, greater than at or about 96%, greater than about or about 97%, greater than at or about 98%, greater than at or about 99%, or at or about 100%. In some embodiments, the provided methods result in improved precision for detection or determination of reverse-transcribed SIN viral nucleic acids, or determination of vector copy number therefrom, in transduced cells, in particular compared to a standard assay involving a qPCR based method for an amplicon also present on a plasmid residual (e.g. WPRE).

In some embodiments, the provided methods result in reproducibility of the method for detection or determination of reverse-transcribed SIN viral nucleic acids, or determination of vector copy number therefrom, in transduced cells that is greater than at or about 90%, such as greater than at or about 95%, greater than at or about 96%, greater than about or about 97%, greater than at or about 98%, greater than at or about 99%, or at or about 100%. In some embodiments, the provided methods result in improved reproducibility for detection or determination of reverse-transcribed SIN viral nucleic acids, or determination of vector copy number therefrom, in transduced cells, in particular compared to a standard assay involving a qPCR based method for an amplicon also present on a plasmid residual (e.g. WPRE).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “amplicon” refers to an amplified nucleic acid product of a PCR reaction or other nucleic acid amplification process. For instance, an amplicon is produced by PCR amplification of a sample comprising nucleic acid in the presence of a nucleic acid polymerase and a specific primer pair. For purposes herein, the amplicon is composed of a nucleotide sequence derived from reverse-transcribed DNA from a self-inactivating viral nucleic acid, such as retroviral nucleic acid. For instance, the amplicon is not produced from viral vector transduction residuals, such as plasmids, that are episomal in a cell transduced with a viral vector. Typically, an amplicon is a DNA amplicon generated by PCR, such as RT-PCR.

As used herein, the term “oligonucleotide” refers to linear oligomers of natural or modified monomers or linkages, including deoxyribonucleosides, ribonucleosides, and the like, capable of specifically binding to a target polynucleotide by way of a regular pattern of monomer-to-monomer interactions, such as Watson-Crick type base pairing. For purposes herein, the term oligonucleotide includes both oligonucleotide probes and oligonucleotide primers.

As used herein, the term “primer” refers to an oligonucleotide that hybridizes to the template strand of a nucleic acid and initiates synthesis of a nucleic acid strand complementary to the template strand when placed under conditions in which synthesis of a primer extension product is induced. Such conditions include the presence of nucleotides and a polymerization-inducing agent such as a DNA or RNA polymerase and at suitable temperature, pH, metal concentration, and salt concentration. For instance, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. As employed herein, an oligonucleotide primer can be naturally occurring, as in a purified restriction digest, or can be produced synthetically. The primer is preferably single-stranded for maximum efficiency in amplification. In some cases, a primer may alternatively be double-stranded. If double-stranded, the primer can first be treated to separate its strands before being used to prepare extension products. This denaturation step is typically effected by heat, but may alternatively be carried out using alkali, followed by neutralization.

As used herein, “primer pair” refers to two primers, a forward primer and a reverse primer, that are capable of participating in PCR amplification of a segment of nucleic acid in the presence of a nucleic acid polymerase to produce a PCR amplicon. It is understood that the orientation of the primers is the direction in which the elongation of the primer in DNA synthesis occurs. Since DNA synthesis is 5′ to 3′, the 3′ ends of a PCR primer set (e.g. forward primer and reverse primer) point towards each other, when they are annealed to their template strand, and the primers anneal on opposite strands of the PCR template. For instance, the forward primer may anneal to (i.e. is complementary to) the minus template minus (−) strand and the reverse primer anneals to (i.e. is complementary to) the template (+) strand. In the case of a retrovirus, such as a lentivirus, genomic RNA is plus-stranded such that the genome and mRNA molecules are produced from the same DNA strand. In some aspects, the primer for the reverse-transcription mediated synthesis of the first DNA strand (i.e., the minus strand) is a host tRNA that is complementary to a primer binding site (PBS) sequence near the 5′ end of the viral RNA. Minus-strand transfer occurs between the R sequences at both ends of the viral genome, thereby allowing minus-strand DNA synthesis to continue accompanied by RNA degradation. In some aspects, the ppt purine rich sequence adjacent to U3 serves as the primer for the synthesis of plus-strand DNA. Plus-strand synthesis continues until RNase H removes the tRNA primer, which in turn allows for the plus-strand transfer and subsequent extension.

As used herein, a “detectable moiety” or “detectable label” refers to a molecule capable of detection, including, but not limited to, radioactive isotopes, fluorophores, chemiluminescers, chromophores, enzymes, enzyme substrates, enzyme co factors, enzyme inhibitors, semiconductor nanoparticles, dyes, metal ions, metal sols, ligands (e.g., biotin, streptavidin or haptens) and the like. The term “fluorescent moiety” refers to a substance or a portion thereof which is capable of exhibiting fluorescence in the detectable range. As used herein, “fluorophore” refers to a fluorescent reporter molecule which, upon excitation with a laser, tungsten, mercury or xenon lamp, or a light emitting diode, releases energy in the form of light with a defined spectrum. Through the process of fluorescence resonance energy transfer (FRET), the light emitted from the-fluorophore-can excite a second molecule whose excitation spectrum overlaps the emission spectrum of the fluorophore. The transfer of emission energy of the fluorophore to another molecule quenches the emission of the fluorophore. The second molecule is known as a quencher molecule. The term “fluorophore” is used interchangeably herein with the term “fluorescent reporter”.

As used herein “quencher” or “quencher molecule” refers to a molecule that, when linked to a fluorescent probe comprising a fluorophore, is capable of accepting the energy emitted by a fluorophore, thereby quenching the emission of the fluorophore. A quencher can be fluorescent, which releases the accepted energy as light, or non-fluorescent, which releases the accepted energy as heat, and can be attached at any location along the length of the probe.

As used herein, “probe” refers to an oligonucleotide that is capable of forming a duplex structure with a sequence in a target nucleic acid, due to complementarity of at least one sequence of the probe with a sequence in the target region, or region to be detected. The term “probe” includes an oligonucleotide as described above, with or without a fluorophore and a quencher molecule attached. The term “fluorescent probe” refers to a probe comprising a fluorophore and a quencher molecule.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell. Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid. Typically, a lentiviral vector contains sufficient lentiviral geneic information to allow packaging of an RNA genome, in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

The term “self-inactivating vector” or “SIN” with reference to a viral vector or retroviral vector refers to a viral vector that is self-inactivating, which is achieved by deleting a portion of the 3′ LTR in retroviral or lentiviral systems to ablate promoter and/or enhancer function. In some embodiments, the U3 is nonfunctioning, such as by deleting the transcription enhancers or the enhancers and promoter in the U3 region of the 3′ LTR. After a round of vector reverse transcription, these changes are copied into the 5′ LTRs to produce a transcriptionally inactive vector genome and thus to prevent vector transcription beyond the first round of replication, ending the life cycle of the virus. The vector is able to infect and integrate into the host genome at most only once.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 2. Amino acid substitutions may be introduced into an antibody of interest and the products screened for a desired activity, for example, retained/improved binding.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a viral vector, refers to an agent that is neither comprised by nor encoded in the corresponding wild-type virus made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### II. Detection of Amplicons

Provided herein are methods of amplifying, detecting and/or quantifying a reverse-transcribed amplicon in a sample, such as a test sample. In some embodiments, the reverse-transcribed amplicon is a reverse-transcriptase dependent amplicon. In some embodiments, the methods can be used to determine vector copy number of a viral vector that has been introduced (e.g. transduced) into a recipient or host cell. In some of any embodiments, the vector is a viral vector, such as a retroviral vector. In some embodiments, the test sample comprises a cell that has been transduced with a viral vector, such as a self-inactivating retroviral vector.

In some embodiments, the methods involve detecting and/or quantifying a viral nucleic acid in a sample, e.g., a sample comprising one or more cells suspected or being transduced and/or one or more cell comprising a viral nucleic acid. In some aspects, the methods provided herein can be used to assess a viral nucleic acid, such as comprised in a viral vector, or to assess vector copy number at various time points before, during, or after, transduction of a cell. In some embodiments, the cell is transduced with a self-inactivating vector, such as a self-inactivating retroviral vector. In some embodiments, the cell is transduced with an HIV-derived vector.

In some embodiments, the provided methods can be used to assess integration, and to detect and/or quantify a viral nucleic acid in a sample. In some embodiments, the provided methods can distinguish detection of reverse-transcribed viral DNA from residual nucleic acids, including vector plasmids, or linear complementary DNA.

In some embodiments, provided are methods for assessing genomic integration of a viral DNA from DNA isolated from one or more cells. In some embodiments, the one or more cells contain, or are suspected to contain, a viral nucleic acid. In some embodiments, the one or more cells are transduced. In some embodiments, the viral nucleic acid further comprises a nucleic acid encoding an exogenous agent. In some aspects, the exogenous agent sequence is or is to be integrated into the genome of the cell. In some embodiments, the exogenous agent includes a nucleic acid sequence encoding a recombinant protein, including regulatory elements, e.g., promoters, transcriptional and/or post-transcriptional regulatory elements or response elements, or markers, e.g., surrogate markers. In some aspects, the methods involve detecting and/or quantifying a reverse-transcriptase dependent amplicon integrated into the genome of the one or more cell, for example, by quantitative methods such as quantitative polymerase chain reaction (qPCR), digital PCR (dPCR) or droplet digital PCR (ddPCR).

In some embodiments, provided are methods for assessing genomic integration of a viral DNA from DNA isolated from a population of cells, said population of cells comprising a plurality of cells that each comprise, or are suspected of comprising, a viral DNA; and determining the average or mean copy number per diploid genome of the reverse-transcribed amplicon, such as a reverse-transcriptase dependent amplicon, sequence integrated into the genome of the plurality of cells. In some embodiments, prior to the assessing, a nucleic acid encoding an exogenous agent has been introduced into at least one of the plurality of cells.

### A. Sample

In some embodiments, the provided methods can be performed on DNA from a sample to amplify or detect a reverse-transcribed amplicon, such as a reverse-transcriptase dependent amplicon. In some embodiments, the methods provided herein also can be used to assess viral copy number in the sample. In some embodiments, the provided methods can be performed on DNA from a sample comprising one or more cells suspected of comprising a viral nucleic acid. In some embodiments, the cells of the sample have been modified (e.g. transduced) with a viral vector, such as a SIN viral vector. In some embodiments, the SIN viral vector is a recombinant viral vector, such as a recombinant retroviral (e.g. lentiviral) vector, that includes sufficient viral (e.g. lentiviral) genetic information to allow packaging of an RNA genome, in the presence of one or more packaging components, into a viral particle capable of infecting a target cell. Infection of the target call may include reverse transcription and, in some cases, integration into the target cell genome. In some embodiments, the SIN viral vector is a recombinant retroviral (e.g. lentiviral) vector that also carries non-viral coding sequences (also referred to herein as “exogenous nucleic acid”) which are to be delivered by the vector to the target cells.

In some embodiments, the methods provided herein can be performed on cells that are genetically engineered via introduction of an exogenous nucleic acid encoding an exogenous agent (i.e. transgene) by infection (e.g. transduction) with the viral vector. In some embodiments, the engineering is carried out by introducing a viral vector containing an exogenous nucleic acid or transgene for integration of the nucleic acid sequence into the genome of the cell, e.g., engineered cell. In some embodiments, the nucleic acid is comprised in a viral vector, such a SIN viral vector for the introduction of the polynucleotide into the cell.

In some embodiments, the cell is transduced with a SIN viral vector particle, e.g. a lentiviral vector. In some embodiments, the vector is replication deficient. In certain embodiments, the viral vector is pseudotyped, e.g. expression of foreign viral genes and/or proteins, for example to alter tropism and targeting and/or modulate the stability of the viral vector particle.

The methods can be carried out on any DNA from any cell that is known or suspected of containing SIN viral nucleic acids. Exemplary features of a SIN viral vector are described known to a skilled artisan. For instance, typically the 5′ LTR and/or the 3′ LTR of a SIN viral vector has been modified. Modification of the 3′ LTR can be used to render the virus replication-defective, that is to say that the virus is not capable of complete replication such that infective virions are not produced. For example the U3 region may be a modified to a non-functional U3 region. The U3 region may be modified e.g. by deletion, truncation, substitution or insertion. The U3 region may be modified by replacing the U3 region with a heterologous promoter to drive transcription of the viral genome during production of viral particles. In some embodiments, the provided methods can be performed on DNA from a sample from one or more cells suspected of comprising a viral nucleic acid from a SIN viral vector (e.g. LV). In some embodiments, the provided methods can be performed on DNA from a sample from one or more cells transduced with a SIN viral vector (e.g. LV), such as any viral vector as disclosed in Section III.

In some embodiments, the SIN viral vector is a retroviral vector. In some embodiments, the retroviral vector includes those derivable from a retrovirus including, but not limited to murine leukemia virus (MLV), human immunodeficiency virus (HIV), equine infectious anemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other Retroviridae including lentiviruses. A detailed list of retroviruses may be found in Coffin et al (“Retroviruses” 1997 Cold Spring Harbour Laboratory Press Eds: J M Coffin, SM Hughes, HE Varmus pp 758-763). In particular embodiments, the retroviral vector is derivable from a lentivirus.

Lentiviral vectors are major tools for gene delivery, providing efficient transduction of a wide variety cell types. The advantages of lentiviral vectors over other systems are the ability to infect both dividing and non-dividing cells in vivo and in vitro and their greater packaging capacity that enables the expression of larger RNA transcripts. The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). Hybrid lentiviruses can also be engineered, such as viruses which contain nucleic acid or protein sequences from each of two primate lentiviruses (i.e., HIV/SIV) (see, e.g., WO2021183761; Uchida et al., J. Virol, 2009, 9854-9862).

The provided methods can be used on any SIN viral vector, such as any retroviral vector such as any lentiviral vector, that undergo reverse transcription involving a transfer of the LTRs to duplicate the LTRs. Typically, these include any SIN viral vector that have been constructed by deleting the transcriptional enhancers or the enhancers and promoter in the U3 region of the 3′ LTR. After a round of vector reverse transcription and, in some cases integration, these changes are copied into both the 5′ and the 3′ LTRs producing a transcriptionally inactive provirus.

Viral proteins involved in early stages of replication include Reverse Transcriptase and Integrase. Reverse Transcriptase is the virally encoded RNA-dependent DNA polymerase. The enzyme uses the viral RNA genome as a template for the synthesis of a complementary DNA copy. Reverse transcriptase also has RNaseH activity for destruction of the RNA-template. Integrase binds both the viral cDNA generated by reverse transcriptase and the host DNA. Integrase processes the LTR before inserting the viral genome into the host DNA. Tat acts as a trans-activator during transcription to enhance initiation and elongation. The Rev responsive element acts post-transcriptionally, regulating mRNA splicing and transport to the cytoplasm.

In some embodiments, the SIN viral vector such as a retroviral vector is an integrating vector in which viral nucleic acids and, in some cases also an exogenous nucleic acid of interest, is integrated into the genome of a recipient cell that is transduced. In other embodiments, the retroviral vectors are non-integrating vectors, such as any as described in WO2007/071994.

In some embodiments, the provided methods can be performed on a cell or a plurality of cells that has been subject to some or all of the steps of transduction, including transduction with a viral vector. In some embodiments, the provided methods are carried out during one or more time points, such as during or after one or more of the steps of transduction. In some embodiments, the provided methods are carried out at the completion of a transduction protocol. In some embodiments, the cell that is suspected of comprising a viral nucleic acid is assessed by any of the methods provided on or at 0, 1, 2, 3, 4, 5, 6, 7, 8, 9, 10, or more days after introduction of the nucleic acids, e.g., via transduction. In some embodiments, the one or more cells suspected of being transduced is assessed by any of the methods provided on or at 0, 1, 2, 3, 4, 5, 6, 7, 8, 9, 10, or more days after introduction of the nucleic acids, e.g., via transduction.

In some embodiments, the provided methods can be performed on a cell that has been genetically modified, i.e., transduced. In some embodiments, transduction is carried out in vivo, such as by administering a viral vector (e.g., Section III) to a subject. In some embodiments, the transduction is carried out ex vivo on primary cells isolated from a subject. In some embodiments, the transduction in in vitro.

In some embodiments, the cells that are transduced and assessed according to the provided methods are primary cells, such as cells obtained or isolated from a subject. The cells can be any cell that is known to have been transduced, or is capable of or suspected of having been transduced, by a viral vector. In some embodiments, the transduction or potential transduction may be ex vivo after isolation of cells from the subject. In some embodiments, the transduction or potential transduction is in vivo in the subject and primary cells or tissues have been isolated from the subject for analysis or assessment. It is within the level of a skilled artisan to appropriately identify or isolate a source of cells that have been or are suspected of having been transduced with a viral vector. In some embodiments, the cells are neoplastic or tumor cells, cancer cells, virus-infected cells, stem cells, central nervous system (CNS) cells, hematopoietic stem cells (HSCs), or fully differentiated cells. In some embodiments, the cells that are transduced are muscle cells (e.g., skeletal muscle cells), kidney cells, liver cells (e.g. hepatocytes), or cardiac cells (e.g. cardiomyocytes). In some embodiments, the cardiac cell is a quiescent cardiomyocyte or a cardiac fibroblast. In some embodiments, the liver cell is a hepatoblast (e.g., a bile duct hepatoblast).

In some embodiments, the cells are immune cells. In some embodiments, the cells are neutrophils, eosinophils, basophils, mast cells, monocytes, macrophages, dendritic cells (DCs), natural killer (NK) cells, or lymphocytes (B cells and T cells). In some embodiments, the cells are T cells. In some embodiments, the T cells are CD4+ T cells or are CD8+ T cells. In some embodiments, the T cells are tumor-infiltrating lymphocytes (TILs). In some embodiments, the B cells are CD20+ B cells or CD19+ B cells. In some embodiments, the NK cells is a NKG2D+ natural killer cell,

In some embodiments, the cells are CD34+ hematopoietic stem cells, CD105+ hematopoietic stem cells, CD117+ hematopoietic stem cells, CD105+ endothelial cells, CD133+ cancer cells, EpCAM+ cancer cells, CD19+ cancer cells, Her2/Neu+ cancer cells, GluA2+ neurons, GluA4+ neurons, a SLC1A3+ astrocyte, a SLC7A10+ adipocyte, or a CD30+ lung epithelial cell.

In some embodiments, cells of the sample include lymphocytes (e.g. T cells and/or NK cells) and are genetically modified (i.e., transduced) with prior activation or stimulation. In some embodiments, the cells for transduction may be a T cell or an NK cell and the activation or stimulation may be achieved by incubation with a stimulatory agent(s), such as an anti-CD3 antibody. In some cases, activation or stimulation may further include incubation with a stimulatory agent that is an antibody against a costimulatory receptor, such as CD28. For instance, the stimulatory agents may include an anti-CD3 antibody and an anti-CD28 antibody. The antibody may include a full-length antibody or an antigen-binding fragment such as a Fab or an scFv, e.g., anti-CD3 Fab and/or anti-CD28 Fab. In some embodiments, the stimulatory agent(s) is present in the reaction mixture where initial contacting of a SIN viral vector and cells (e.g., lymphocytes including T and/or NK cells) occurs. For example, such stimulatory agent(s) can be in solution in the reaction mixture. For example, soluble anti-CD3 antibodies, and in some cases also anti-CD28 antibodies, can be present in the reaction mixture during the contacting and optional further incubation thereafter, at 25-200, 50-150, 75-125, or 100 ng/ml. In some embodiments, the stimulatory agent(s) element is associated with or bound to a solid surface, such as to a bead (e.g. magnetic bead) or immobilized on the surface of a plate. In still other embodiments, the stimulatory agents(s) is associated with the retroviral vector surface. Accordingly, in some embodiments, the SIN viral vector (e.g., a self-inactivating retroviral vector of Section III) can further include a stimulatory agent(s) element, which in further some embodiments is associated with the external side of the surface of the retroviral vector.

In some embodiments, activation by elements that are not present on the vector surface (i.e., self-inactivating retroviral vector) is required for modifying, genetically modifying, and/or transducing the cell. In some embodiments, at least 50%, 60%, 70%, 75%, 80%, 90%, 95%, or 99% or all of the cells (i.e., cells of the sample, e.g., T and/or NK cells) are activated when they are combined with retroviral vector, and typically are not resting when they are contacted with vector (i.e., self-inactivating retroviral vector).

In some embodiments, the activation of the cell is for no more than 15 minutes, 30 minutes, 1, 2, 4, or 8 hours before the contacting of the viral vector with cells (i.e. cells of the sample, e.g. T and/or NK cells). In some embodiments, at most 25%, 30%, 35%, 40%, 45%, or 50%, or all of the cells (i.e., cells of the sample, e.g., T and/or NK cells) are resting when they are combined with retroviral vector.

In certain embodiments, cells of the sample include lymphocytes (e.g., T cells and/or NK cells) and the cells are genetically modified (i.e., transduced) without prior activation or stimulation, whether in vivo, in vitro, or ex-vivo. In some embodiments, cells of the sample are genetically modified (i.e., transduced) without ex vivo or in vitro activation or stimulation after an initial contacting with or without an optional incubation, or without requiring ex vivo or in vitro activation or stimulation after an initial contacting with or without an optional incubation.

In some embodiments, the sample cells consist of 0-10% resting cells, such as resting T cells and/or NK cells, for example as determined by Ki-67. In some embodiments, the cells that are contacted by self-inactivating retroviral vector include between 10, 9, 8, 7, 6, and 5% resting cells on the high end of the range and 4, 3, 2, 1, or 0% resting cells on the low end of the range. In some embodiments, the cells (i.e., T and/or NK cells) include naive cells.

In some embodiments, the cell is activated during the contacting with viral vector, such as those described in Section III. In some embodiments, activation by elements that are not present on the vector surface (i.e., self-inactivating retroviral vector) is not required for modifying, genetically modifying, and/or transducing the cell. Accordingly, such activation or stimulation elements are not required other than on the vector, before, during, or after the contacting. In some embodiments, at least 25%, 50%, 60%, 70%, 75%, 80%, 90%, 95%, or 99%, or all of the cells (i.e., cells of the sample, e.g., T and/or NK cells) are resting when they are combined with retroviral vector, and typically are resting when they are contacted with vector (i.e., self-inactivating retroviral vector). For instance, in methods in which lymphocytes such as T cells and/or NK cells are primary cells from blood or a component thereof (e.g., using a sample isolated from a patient), cells can be contacted in the typically resting state they were in when present in the collected blood in vivo immediately before collection. In some embodiments, the sample cells are composed of between 95 and 100% resting cells (determined by Ki-67). In some embodiments, the cells that are contacted by self-inactivating retroviral vector include between 80, 85, 90, 91, 92, 93, 94, and 95% resting cells on the low end of the range and 96, 97, 98, 99, or 100% resting cells on the high end of the range. In some embodiments, the cells (i.e., T and/or NK cells) include naive cells.

In some aspects, a viral vector such as a SIN viral vector, e.g. as described in Section III, is contacted with cells of a sample (e.g. containing T cells and/or NK cells, with or without prior activation or stimulation) to transduce the cells. In the methods, the period of contact is sufficient to permit binding or fusion of the SIN viral vector, such as a retroviral vector (e.g., a replication incompetent self-inactivating retroviral vector of Section III) with cells of the sample. Genetic material from the SIN viral vector (e.g., a replication incompetent self-inactivating retroviral vector of Section III) enters the cells at which time the cells are “genetically modified. In some aspects, such process might occur hours or even days after the contacting is initiated, and even after non-associated retroviral vector particles are rinsed away. Then the genetic material is typically integrated into the genomic DNA of the cells (e.g., T cells and/or NK cells), at which time the cells are now “transduced.” In some aspects, integration of viral nucleic acids or genetic material therefrom into the genomic DNA of a cell results in stable transfection of the cell.

In aspects of the methods of transducing or genetically modifying cells (e.g., lymphocytes, such as T cells and/or NK cells), genetic material from a SIN viral vector may include transgene sequences that include polynucleotides encoding an exogenous agent, such as is described in Section III.C. For example, an exogenous agent can include a therapeutic agent, a chimeric antigen receptor (CAR), a lymphoproliferative element or other agents. For example, in some embodiments, lymphoproliferative elements can be delivered from the genome of the retroviral vector inside genetically modified, and/or transduced cells (e.g. T cells and/or NK cells), such that those cells have the characteristics of increased proliferation and/or survival.

In some embodiments, the contacting is carried out for between 15 minutes and 6 days, such as for between 15 minutes and 4 days, between 15 minutes and 2 days, between 15 minutes and 24 hours, between 15 minutes and 12 hours, between 15 minutes and 6 hours, between 15 minutes and 2 hours, between 15 minutes and 1 hour, or between 15 minutes and 30 minutes. In some embodiments, the contacting is carried out ex vivo or in vitro. In some embodiments, the contacting is carried out in vivo. Methods of retroviral and lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. Throughout this disclosure, a transduced, or in some embodiments a stably transfected, cell of the sample (e.g., a T cell and/or NK cell) includes progeny of ex vivo transduced cells that retain at least some of the viral nucleic acids that are incorporated into the genome of a cell during the transduction.

The contacting step of a method for transducing and/or a method for genetically modifying cells (e.g., lymphocytes, such as T cells and/or NK cells) typically includes an initial step in which the retroviral vector, typically a population of retroviral vectors, are brought into contact with cells. In some embodiments, the contacting is performed with a population of blood cells and viral vectors while in suspension in a liquid buffer and/or media to form a transduction reaction mixture. This contacting, as in other aspects provided herein, can be followed by an optional incubating period of this reaction mixture. In some embodiments, incubation can be at between 23 and 39° C., and in certain embodiments at 37° C. In certain embodiments, incubation of the transduction reaction can be carried out at 37-39° C. for faster fusion/transduction.

In some embodiments, retroviral vector that remain unassociated in suspension are removed from the reaction mixture, for example by washing the reaction mixture over a filter, such as a leukoreduction filter, that retains cells (i.e., leukocytes including T cells and NK cells) but not free, unassociated viral particles. The cells and retroviral vector when brought into contact in the transduction reaction mixture can be immediately processed to remove the retroviral vector that remain free in suspension and not associated with cells, from the cells. Optionally, the cells in suspension and retroviral vector whether free in suspension or associated with the cells in suspension, can be incubated for various lengths of time, as provided herein for a contacting step in a method provided herein. Before further steps, a wash can be performed. Such suspension can include allowing cells and retroviral vector to settle or causing such settling through application of a force, such as a centrifugal force, to the bottom of a vessel or chamber.

Illustrative methods are disclosed herein for modifying cells (i.e., lymphocytes). In some embodiments, the contacting step in any method provided herein of transducing, genetically modifying, and/or modifying a cell of the sample, including a T cell and/or an NK cell, can be performed (or can occur) for any of the time periods provided in this specification. For example, said contacting can be for less than 24 hours, for example, less than 12 hours, less than 8 hours, less than 4 hours, less than 2 hours, less than 1 hour, less than 30 minutes or less than 15 minutes. In each case there is at least an initial contacting step in which retroviral vector (i.e., a replication incompetent self-inactivating retroviral vector of Section III) and cells come into contact in suspension in a transduction reaction mixture before retroviral vector that remain in suspension not associated with a cell, are separated from cells and typically discarded. Without wishing to be bound by theory, is believed that contacting begins at the time that retroviral vector and cells are combined together, typically by adding a solution containing the retroviral vector into a solution containing cells (e.g., lymphocytes such as T cells and/or NK cells).

After initial contacting, in some embodiments there is an incubating of the reaction mixture containing cells and retroviral vector (i.e., a replication incompetent self-inactivating retroviral vector of Section III), in suspension for a specified time period without removing retroviral vector that remain free in solution and not associated with cells. Thus, in some embodiments, the contacting (including initial contacting and optional incubation) can be performed (or can occur) for between 15 minutes and 12 hours, between 15 minutes and 10 hours, or between 15 minutes and 8 hours. In certain embodiments that comprise a cold contacting step, a secondary incubation is performed by suspending cells after an optional wash step such that retroviral vector, that are not associated with a cell are washed away. In some embodiments, the secondary incubation is performed at temperatures between 32° C. and 42° C., such as at 37° C. The optional secondary incubation can be performed for any length of time discussed herein. In some embodiments, the optional secondary incubation is performed for 6 hours or less. Thus, in some embodiments, the contacting (including initial contacting and optional incubation) can be performed (or can occur) (where as indicated in general herein the low end of a selected range is less than the high end of the selected range) for between 30 seconds or 1, 2, 5, 10, 15, 30, or 45 minutes, or 1, 2, 3, 4, 5, 6, 7, or 8 hours on the low end of the range, and between 10 minutes, 15 minutes, 30 minutes, or 1, 2, 4, 6, 8, 10, 12, 18, 24, 36, 48, and 72 hours. Thus, in some embodiments, after the time when a reaction mixture is formed by adding retroviral vector to cells (i.e., lymphocytes), the reaction mixture can be incubated for between 5 minutes on the low end of the range and 10, 15, or 30 minutes or 1, 2, 3, 4, 5, 6, 8, 10 or 12 hours. In some embodiments, the reaction mixture can be incubated for between 15 minutes and 12 hours, 15 minutes and 10 hours, 15 minutes and 8 hours, 15 minutes and 6 hours, 15 minutes and 4 hours, 15 minutes and 2 hours, 15 minutes and 1 hour, 15 minutes and 45 minutes, or 15 minutes and 30 minutes. In some embodiments, the reaction mixture can be incubated for between 30 minutes and 12 hours, 30 minutes and 10 hours, 30 minutes and 8 hours, 30 minutes and 6 hours, 30 minutes and 4 hours, 30 minutes and 2 hours, 30 minutes and 1 hour, or 30 minutes and 45 minutes. In some embodiments, the reaction mixture can be incubated for between 1 hour and 12 hours, 1 hour and 8 hours, 1 hour and 4 hours, or 1 hour and 2 hours. In another embodiment, the contacting is performed for between an initial contacting step only (without any further incubating in the reaction mixture including the retroviral vector free in suspension and cells in suspension) without any further incubation in the reaction mixture, or a 5 minute, 10 minute, 15 minute, 30 minute, or 1 hour incubation in the reaction mixture.

After the indicated time period for the initial contacting and optional incubation that can be part of the contacting step, cells are separated from retroviral vector that are not associated with such cells. For example, this can be performed using a PBMC enrichment procedure (e.g., a Ficoll gradient in a Sepax unit), or in certain illustrative embodiments provided herein, by filtering the reaction mixture over a cell filter, such as a leukocyte depletion filter set assembly, and then collecting the cells (i.e., leukocytes which include T cells and NK cells). In another embodiment, this can be performed by centrifugation of the reaction mixture at a relative centrifugal force less than 500 g, for example 400 g, or between 300 and 490 g, or 350 and 450 g. Such centrifugation to separate retroviral vector from cells can be performed for example, for between 5 minutes and 15 minutes, or between 5 minutes and 10 minutes. In illustrative embodiments where centrifugal force is used to separate cells from retroviral vector that are not associated with cells, such g force is typically lower than the g forces used successfully in spinoculation procedures.

In some illustrative embodiments, a method provided herein in any aspect, does not involve performing a spinoculation. In such embodiments, the cell or cells are not subjected to a spinoculation of at least 400 g, 500 g, 600 g, 700 g, or 800 g for at least 15 minutes. In some embodiments, the cell or cells are not subjected to a spinoculation of at least 800 g for at least 10, 15, 20, 25, 30, 35, 40, or 45 minutes.

In some embodiments, spinoculation is included as part of a contacting step. In some embodiments, when spinoculation is performed there is no additional incubating as part of the contacting, as the time of the spinoculation provides the incubation time of the optional incubation discussed above. In other embodiments, there is an additional incubation after the spinoculating of between 15 minutes and 4 hours, 15 minutes and 2 hours, or 15 minutes and 1 hour. The spinoculation can be performed for example, for 30 minutes to 120 minutes, typically for at least 60 minutes, for example for 60 minutes to 180 minutes, or 60 minutes to 90 minutes. The spinoculation is typically performed in a centrifuge with a relative centrifugal force of at least 800 g, and more typically at least 1200 g, for example between 800 g and 2400 g, 800 g and 1800 g, 1200 g and 2400 g, or 1200 g and 1800 g. After the spinoculation, such methods typically involve an additional step of re-suspending the cells and retroviral vector, and then removing retroviral vector that are not associated with cells according to steps discussed above when spinoculation is not performed.

The contacting step including the optional incubation therein, and the spinoculation, in embodiments that include spinoculation, can be performed at between 4° C. and 42° C. or 20° C. and 37° C. In certain illustrative embodiments, spinoculation is not performed and the contacting and associated optional incubation are carried out at 20-25° C. for 4 hours or less, 2 hours or less, 1 hour or less, 30 minutes or less, 15 minutes or less, or 15 minutes to 2 hours, 15 minutes to 1 hour, or 15 minutes to 30 minutes.

Media that can be included in a contacting step, for example when the cells and retroviral vector (i.e., a replication incompetent self-inactivating retroviral vector of Section III) are initially brought into contact, or in any aspects provided herein, during optional incubation periods with the reaction mixture thereafter that include retroviral vector and cells in suspension in the media, or media that can be used during cell culturing and/or during various wash steps in any aspects provided herein, can include base media such as commercially available media for ex vivo cell (i.e., T cell and/or NK cell) culture. Non-limiting examples of such media include, X-VIVO™ 15 Chemically Defined, Serum-free Hematopoietic Cell Medium (Lonza) (2018 catalog numbers BE02-060F, BE02-00Q, BE-02-061Q, 04-744Q, or 04-418Q), ImmunoCulf™-XF T Cell Expansion Medium (STEMCELL Technologies) (2018 catalog number 10981), PRIME-XV® T Cell Expansion XSFM (Irvine Scientific) (2018 catalog number 91141), AIM V® Medium CTS™ (Therapeutic Grade) (Thermo Fisher Scientific (Referred to herein as “Thermo Fisher”), or CTS™ Optimizer™ media (Thermo Fisher) (2018 catalog numbers A10221-01 (basal media (bottle)), and A10484-02 (supplement), A10221-03 (basal media (bag)), A1048501 (basal media and supplement kit (bottle)) and, A1048503 (basal media and supplement kit (bag)). Such media can be a chemically defined, serum-free formulation manufactured in compliance with cGMP, as discussed herein for kit components. The media can be xeno-free and complete. In some embodiments, the base media has been cleared by regulatory agencies for use in ex vivo cell processing, such as an FDA 510(k) cleared device.

In some embodiments, the media is the basal media with the supplied T cell expansion supplement of 2018 catalog number A1048501 (CTS™ OpTmizer™ T Cell Expansion SFM, bottle format) or A1048503 (CTS™ OpTmizer™ T Cell Expansion SFM, bag format) both available from Thermo Fisher (Waltham, MA). Additives such as human serum albumin, human AB+ serum, and/or serum derived from the subject can be added to the transduction reaction mixture. In some embodiments, supportive cytokines can be added to the transduction reaction mixture, such as IL2, IL7, or IL 15, or those found in human sera. dGTP can be added to the transduction reaction.

In some embodiments of any method herein that includes a step of genetically modifying cells (e.g., T cells and/or NK cells), the cells have not been incubated on a substrate that adheres to monocytes for more than 4 hours in one embodiment, or for more than 6, hours in another embodiment, or for more than 8 hours in another embodiment before the transduction. In one embodiment, the cells are lymphocytes and a sample containing lymphocytes (e.g., T cells and/or NK cells) have been incubated overnight on an adherent substrate to remove monocytes before the transduction. In another embodiment, the method can include incubating the cells on an adherent substrate that binds monocytes for no more than 30 minutes, 1 hour, or 2 hours before the transduction. In another embodiment, the cells are lymphocytes and a sample containing lymphocytes (e.g. T cells and/or NK cells) are exposed to no step of removing monocytes by an incubation on an adherent substrate before said transduction step. In another embodiment, the cells (e.g. T cells and/or NK cells) are not incubated with or exposed to a bovine serum, such as a cell culturing bovine serum, for example fetal bovine serum before or during a contacting step and/or a modifying and/or a genetically modifying and/or transduction step.

Some or all of the steps of the methods for modifying provided herein, or uses of such methods, are performed in a closed system. Thus, reaction mixtures formed in such methods, and genetically modified, and/or transduced cells (e.g., lymphocytes, such as T cells and/or NK cells) made by such methods, can be contained within such a closed system. A closed system is a cell processing system that is generally closed or fully closed to an environment, such as an environment within a room or even the environment within a hood, outside of the conduits such as tubes, and chambers, of the system in which cells are processed and/or transported. Such a process is designed and can be operated such that the product is not exposed to the outside environment. Material transfer occurs via sterile connections, such as sterile tubing and sterile welded connections. Air for gas exchange can occur via a gas permeable membrane, via 0.2 pm filter to prevent environmental exposure. In some embodiments, the methods are performed on cells, for example to provide modified and in illustrative embodiments genetically modified T cells.

Such closed system methods can be performed with commercially available devices. Different closed system devices can be used at different steps within a method and the cells can be transferred between these devices using tubing and connections such as welded, luer, spike, or clave ports to prevent exposure of the cells of the sample or media to the environment. For example, the sample comprising cells can comprise blood to be collected into an IV bag or syringe, optionally including an anticoagulant, and in some aspects, transferred to a Sepax 2 device (Biosafe) for PBMC enrichment and isolation. In other embodiments, whole blood can be filtered to collect cells (i.e., leukocytes) using a leukoreduction filter assembly. The isolated cells be transferred to a chamber of a G-Rex device for an optional activation, a transduction and optional expansion. Alternatively, collected blood can be transduced in a blood bag, for example, the bag in which it was collected. Finally, the cells can be harvested and collected into another bag using a Sepax 2 device. The methods can be carried out in any device or combination of devices adapted for closed system cell (i.e., T cell and/or NK cell) production. Non-limiting examples of such devices include G-Rex devices (Wilson Wolf), GatheRex (Wilson Wolf), Sepax 2 (Biosafe), WAVE Bioreactors (General Electric), a CuItiLife Cell Culture bag (Takara), a PermaLife bag (OriGen), CliniMACS Prodigy (Miltenyi Biotec), and VueLife bags (Saint-Gobain). In some embodiments, the optional activating, the transducing, and optional expanding can be performed in the same chamber or vessel in the closed system.

In some embodiments, DNA is isolated from cells of the sample, such as cells known or suspected of containing viral nucleic acids from a SIN viral vector. In some embodiments, DNA is isolated from cells that have been transduced with a SIN viral vector, e.g. as described above. Methods of isolation of genomic DNA are known in the art. In particular, kits for isolation of genomic DNA are commercially available (for example Purelink™ Genomic Kit from Invitrogen or Wizard® Genomic DNA Purification Kit from Promega).

### B. Amplicon Detection

In some of any embodiments, the methods use oligonucleotide reagents (e.g. oligonucleotide primers and probes) or a combination of reagents capable of detecting a reverse-transcriptase dependent amplicon (hereinafter also called a “target amplicon) from a viral nucleic acid present in a sample. In particular embodiments, the methods of detection are carried out in a single assay. In one format, primer pairs and probes capable of detecting reverse-transcribed deoxyribonucleic acid (DNA) from one or more cells suspected of comprising a viral nucleic acid, such as from cells transduced with a viral vector (e.g. SIV), e.g. any as described in Section II.A.

In some embodiments, the primer and/or probe sequence can specifically detect a reverse-transcriptase dependent region containing viral nucleic acid that is heterologous to cells of the sample, thereby producing a reverse-transcriptase dependent amplicon or target amplicon. In some embodiments, the primer and/or probe sequence that can specifically amplify a reverse-transcriptase dependent amplicon sequence is composed of a nucleotide sequence derived from reverse-transcribed DNA from a retroviral nucleic acid that is integrated into the cell genome. For instance, certain primers and probes are from regions that are capable of specifically detected reverse-transcribed viral DNA but not capable of detecting certain RNA that has not been reverse-transcribed in the cell, such as episomal residual plasmids used in connection with methods of generating vector viral particles.

In some embodiments, the primer and/or probe are specific for amplifying nucleic acids of a reverse-transcriptase dependent amplicon include those specific to viral nucleic acid sequences, particularly those present in a retroviral vector, such as a SIV. By way of example, the primers and probes can be specific to a region of viral nucleic acid that has undergone reverse-transcription when integrated into the genome of a cell, such as a region including retroviral long terminal repeat sequences. In some embodiments, the methods can be used to amplify viral nucleic acid within regions that include one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a polypurine tract (PPT), a primer-binding site (PBS), a packaging site (e.g., psi (Ψ)), or RRE (e.g., to bind to Rev and promote nuclear export). In some embodiments, primers and probes are composed of sequences from LTR regions, such as the retroviral 3′ U3 long terminal repeat sequence and the retroviral long terminal repeat sequence that is located downstream of the 5′ U5 sequence. Oligonucleotides for use in the assays described herein can be derived from the 3′ U3 long terminal repeat sequence and retroviral long terminal repeat sequence located downstream of the 5′ U5 sequence. Representative sequences are listed herein.

An amplification method such as PCR can be used to amplify polynucleotides from cDNA derived from genomic RNA after reverse transcription. Thus, in provided aspects if viral nucleic acid is present (e.g., integrated) in the genome of a cell and is reverse transcribed it can be amplified and detected by the provided methods. In some embodiments, the probe and/or primer can be used for exemplary amplification methods, such as quantitative PCR (qPCR) or droplet digital PCR (ddPCR) described below.

### 1. Primers and Probes

Provided herein are primers specific for a region of reverse-transcribed viral DNA to produce a target amplicon. In some embodiments, the target amplicon is detected and/or quantified using the one or more oligonucleotide primers in combination with a probe. In some embodiments, the provided primers and probes are useful for detecting a target viral sequence associated with a SIN viral vector, such as a lentiviral vector, known or suspected of being present in a sample, such as in cells transduced with the SIN viral vector. In some embodiments, the one or more oligonucleotide primers are specific for a portion of a target sequence, i.e. a region of viral nucleic acid that is subject to reverse-transcription when integrated into the genome of a cell. In some embodiments, the provided probes and primers are useful for detecting viral nucleic acid sequences present in reverse-transcribed viral DNA after the process of reverse transcription of the retroviral genome (see e.g. FIG. 1).

In some embodiments, the primers include a forward primer and a reverse primer. In some embodiments, the forward primer and reverse primer are each specific for a region of viral nucleic acid that is known to undergo reverse transcription, such as regions within or near the 3′ or 5′ LTR. In some embodiments, the forward primer and reverse primer are specific for different regions of a reverse-transcribed retroviral nucleic acid sequence.

In some of any provided embodiments, the forward oligonucleotide primer is a sequence of at least 15 contiguous nucleotides (base pairs) on the plus strand present in a first region of the reverse-transcribed SIN viral vector DNA. In some embodiments, the forward oligonucleotide primer has a length of 15-25, 15-22, 15-18, 18-25, 18-22, or 22-25 nucleotides on the plus strand present in a first region of the reverse-transcribed SIN viral vector. In some embodiments, the forward oligonucleotide primer has a length of 15, 16, 17, 18, 19, 20, 21, 22, 23, 24, 25, 26, 27, 28, 29, or 30 contiguous nucleotides on the plus strand present in a first region of the reverse-transcribed SIN viral vector DNA.

In some embodiments, the forward oligonucleotide primer is a plus-stranded sequence in a first region that is within the deleted U3 (delU3) of the reverse-transcribed SIN viral vector DNA. In some embodiments, the deleted U3 region comprises a deletion in any of the U3 modulatory, enhancer, and core promoter domains. It is within the level of a skilled artisan to choose an appropriate primer that is complementary to such a first region within the delU3 depending on the particular SIN viral vector (see e.g. Table 1). In some embodiments, the first region is a contiguous sequence within the delU3 of the SIN viral vector.

In some embodiments, for example, the PCR primers may be a length, such as 18 to 30 nucleotides, that provides for an optimal melting temperature (Tm), annealing temperature (Ta) and/or specificity. In some aspects, the optimal melting temperatures for a primer pair are between 60-64°. It is within the level of a skilled artisan to determine the optimum temperature for enzymatic function based on typical cycling and reaction conditions. In some embodiments, the primers provide for an annealing temperature that ensures specificity of annealing. For instance, annealing temperatures that are too low may result in off target binding (i.e., internal single-base mismatches or partial annealing may be observed). Additionally, the GC content of primers should be considered, as consecutive G residues may result in non-specific binding.

In some embodiment, the first region is a delU3 sequence set forth in any one of SEQ ID NOS: 1 and 14-28 or a sequence having at least or about 85, 90, 91, 92, 93, 94, 95, 96, 97, 98, or 99% sequence identity with any one of SEQ ID NOS: 1 and 14-28.

In some embodiments, the forward primer is a contiguous sequence of nucleotides of the sequence set forth in any one of SEQ ID NOS: 1 and 14-28 or a sequence having at least or about 85, 90, 91, 92, 93, 94, 95, 96, 97, 98, or 99% sequence identity with any one of SEQ ID NOS: 1 and 14-28. In some embodiments, the forward primer is a contiguous sequence of nucleotides of the sequence set forth in any one of SEQ ID NOS: 1 and 14-28. In some embodiments, the forward primer is a contiguous sequence of nucleotides of the sequence set forth in SEQ ID NO: 1 or a sequence having at least or about 85, 90, 91, 92, 93, 94, 95, 96, 97, 98, or 99% sequence identity with such a sequence or portion of such a sequence. In some embodiments, the forward primer is a contiguous sequence of nucleotides of the sequence set forth in SEQ ID NO:1.

In some embodiments, the forward oligonucleotide primer is a nucleotide sequence on the plus strand in a first region that includes a retroviral long terminal repeat sequence that is or includes at least a portion of the conserved U3 attachment (U3 att) sequence. In some embodiments, the forward oligonucleotide primer includes, or includes at least a portion of, the sequence set forth in SEQ ID NO: 46, 47, or 48 or a sequence having at least or about 85, 90, 91, 92, 93, 94, 95, 96, 97, 98, or 99% identity with such a sequence or portion of such a sequence. In some embodiments, the forward oligonucleotide primer includes, or includes at least a portion of, the sequence set forth in SEQ ID NO: 46, 47, or 48. In some embodiments, the forward oligonucleotide primer includes the U3 attachment sequence set forth in SEQ ID NO: 46 or a sequence having at least or about 85, 90, 91, 92, 93, 94, 95, 96, 97, 98, or 99% identity with such a sequence. In some embodiments, the forward oligonucleotide primer includes the U3 attachment sequence set forth in SEQ ID NO: 47 or a sequence having at least or about 85, 90, 91, 92, 93, 94, 95, 96, 97, 98, or 99% identity with such a sequence. In some embodiments, the forward oligonucleotide primer includes the U3 attachment sequence set forth in SEQ ID NO: 48 or a sequence having at least or about 85, 90, 91, 92, 93, 94, 95, 96, 97, 98, or 99% identity with such a sequence.

In some of any provided embodiments, the reverse oligonucleotide primer is a sequence of at least 15 contiguous nucleotides (base pairs) on the minus strand present in a second region of the reverse-transcribed SIN viral vector DNA. In some embodiments, the reverse oligonucleotide primer has a length of 15-25, 15-22, 15-18, 18-25, 18-22, or 22-25 contiguous nucleotides on the minus strand present in a second region of the reverse-transcribed SIN viral vector DNA. In some embodiments, the reverse oligonucleotide primer has a length of 15, 16, 17, 18, 19, 20, 21, 22, 23, 24, 25, 26, 27, 28, 29, or 30 nucleotides that is complementary to a contiguous sequence of base pairs present in a second region of the reverse-transcribed SIN viral vector.

In some embodiments, the reverse oligonucleotide primer is a minus-stranded sequence in a second region that is downstream of the first region in the reverse-transcribed SIN viral vector DNA to make a replicable reverse-transcriptase-dependent amplicon. In some embodiments, the reverse-transcriptase-dependent amplicon is an amplicon that is between 50 and 500 base pairs in length, such as between 50 and 450 base pairs, between 50 and 400 base pairs, between 50 and 350 base pairs, between 50 and 300 base pairs, between 50 and 250 base pairs, between 50 and 200 base pairs, 100 and 500 base pairs in length, such as between 100 and 450 base pairs, between 100 and 400 base pairs, between 100 and 350 base pairs, between 100 and 300 base pairs, between 100 and 250 base pairs, between 100 and 200 base pairs, between 200 and 500 base pairs, between 200 and 450 base pairs, between 200 and 400 base pairs, between 200 and 350 base pairs, between 200 and 300 base pairs, between 200 and 250 base pairs, between 250 and 500 base pairs, between 250 and 450 base pairs, between 250 and 400 base pairs, between 250 and 350 base pairs, between 250 and 300 base pairs, between 300 and 500 base pairs, between 300 and 450 base pairs, between 300 and 400 base pairs, between 300 and 350 base pairs, between 350 and 500 base pairs, between 350 and 450 base pairs, between 350 and 400 base pairs, between 400 and 500 base pairs, between 400 and 450 base pairs or between 450 and 500 base pairs. In some embodiments, the reverse-transcriptase-dependent amplicon is an amplicon that is between 200 and 400 base pairs in length. In some embodiments, the reverse-transcriptase-dependent amplicon is an amplicon that is between 200 and 300 base pairs in length. In some embodiments, the reverse-transcriptase-dependent amplicon is at or about 150, 175, 200, 225, 250, 275, 300, 325, 350, 375 or 400 nucleotides in length or any value between any of the foregoing.

In some embodiments, the reverse oligonucleotide primer is a minus-stranded sequence in a second region that is within at least a portion of the primer binding site (PBS), the IFN-stimulated response element (ISRE), and/or the Psi (Ψ) packaging sequence of the SIN viral vector. In some embodiments, the reverse oligonucleotide primer is a minus-stranded sequence in a second region within at least a portion of the PBS. In some embodiments, the reverse oligonucleotide primer is a minus-stranded sequence in a second region within at least a portion of the ISRE. In some embodiments, the reverse oligonucleotide primer is a minus-stranded sequence in a second region within at least a portion of the Psi (Ψ) packaging sequence. It is within the level of a skilled artisan to choose an appropriate primer that is a minus-stranded sequence in such a second region depending on the particular SIN viral vector (see e.g. Table 1).

In some embodiments, the second region is set forth in any one of SEQ ID NOS: 2 and 29-45 or a sequence having at least or about 85, 90, 91, 92, 93, 94, 95, 96, 97, 98, or 99% sequence identity with any one of SEQ ID NOS: 2 and 29-45.

In some embodiments, the reverse primer is a contiguous sequence of nucleotides of the sequence set forth in any one of SEQ ID NOS: 2 and 29-45 or a sequence having at least or about 85, 90, 91, 92, 93, 94, 95, 96, 97, 98, or 99% sequence identity with any one of SEQ ID NOS: 2 and 29-45. In some embodiments, the reverse primer is a contiguous sequence of nucleotides of the sequence set forth in any one of SEQ ID NOS: 2 and 29-45. In some embodiments, the reverse primer is a contiguous sequence of nucleotides of the sequence set forth in SEQ ID NO: 2 or a sequence having at least or about 85, 90, 91, 92, 93, 94, 95, 96, 97, 98, or 99% sequence identity with such a sequence or portion of such a sequence. In some embodiments, the reverse primer is a contiguous sequence of nucleotides within the sequence set forth in SEQ ID NO:2.

In some embodiments, the forward primer is a contiguous sequence of nucleotides within a first region having the sequence set forth in SEQ ID NO:14 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:29 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:14 and the reverse primer is a contiguous sequence of nucleotides within a second region having the sequence set forth in SEQ ID NO:29. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:15 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:30 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:15 and the reverse primer is a contiguous sequence of nucleotides within a second region having the sequence set forth in SEQ ID NO:30. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:16 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:31 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:16 and the reverse primer is a contiguous sequence of nucleotides within a second region having the sequence set forth in SEQ ID NO:31. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:17 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:32 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:17 and the reverse primer is a contiguous sequence of nucleotides within a second region having the sequence set forth in SEQ ID NO:32. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:18 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:33 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:18 and the reverse primer is a contiguous sequence of nucleotides within a second region having the sequence set forth in SEQ ID NO:33. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:19 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:35 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:19 and the reverse primer is a contiguous sequence of nucleotides within a second region having the sequence set forth in SEQ ID NO:35. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:20 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:36 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:20 and the reverse primer is a contiguous sequence of nucleotides within a second region having the sequence set forth in SEQ ID NO:36. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:21 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:38 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:21 and the reverse primer is a contiguous sequence of nucleotides within a second region having the sequence set forth in SEQ ID NO:38. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:22 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:39 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:22 and the reverse primer is a contiguous sequence of nucleotides within a second region having the sequence set forth in SEQ ID NO:39. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:23 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:40 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:23 and the reverse primer is a contiguous sequence of nucleotides within a second region having the sequence set forth in SEQ ID NO:40. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:24 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:41 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:24 and the reverse primer is a contiguous sequence of nucleotides within a second region having the sequence set forth in SEQ ID NO:41. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:25 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:42 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:25 and the reverse primer is a contiguous sequence of nucleotides within a second region having the sequence set forth in SEQ ID NO:42. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:26 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:43 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:26 and the reverse primer is a contiguous sequence of nucleotides within a second region having the sequence set forth in SEQ ID NO:43. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:27 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:44 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:27 and the reverse primer is a contiguous sequence of nucleotides within a second region having the sequence set forth in SEQ ID NO:44. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:28 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:45 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:28 and the reverse primer is a contiguous sequence of nucleotides within a second region having the sequence set forth in SEQ ID NO:45. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the forward primer is a contiguous sequence of nucleotides within a first region having the sequence set forth in SEQ ID NO:1 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:2 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:1 and the reverse primer is a contiguous sequence of nucleotides within a second region having the sequence set forth in SEQ ID NO:2. In some embodiments, the forward and reverse primer are each independently at least 15 nucleotides in length, such as 15-30 nucleotides in length. In some embodiments, the forward and reverse primers are each between 18-25 nucleotides in length. In some embodiments, the forward and reverse primers are each between 18-22 nucleotides in length.

In some embodiments, the reverse-transcribed amplicon, such as a reverse-transcriptase-dependent amplicon, is one that: (i) has the sequence set forth in SEQ ID NO:10, (ii) is a portion of the sequence of SEQ ID NO:10 that is a replicable amplicon of at least 100, at least 150, at least 200 or at least 250 nucleotides in length, or (ii) is a sequence that has at least 80%, at least 85%, at least 90% or at least 95% sequence identity to (i) or (ii). In some embodiments, the reverse-transcribed amplicon, such as reverse-transcriptase-dependent amplicon, has the sequence set forth in SEQ ID NO:10 or a sequence that has at least 80%, at least 85%, at least 90% or at least 95% sequence identity to SEQ ID NO:10. In some embodiments, the reverse-transcriptase-dependent amplicon is set forth in SEQ ID NO:10.

In some embodiments, the forward oligonucleotide primer comprises the sequence set forth in SEQ ID NO 3, or a sequence having at least or about 85, 90, 91, 92, 93, 94, 95, 96, 97, 98, or 99% identity with a sequence as set forth in SEQ ID NO 3. In some embodiments, the forward oligonucleotide primer comprises the sequence set forth in SEQ ID NO 3 and is at least 15, at least 18, at least 22, at least 25, or at least 30 nucleotides in length. In some embodiments, the forward oligonucleotide primer comprises a sequence having at least or about 85, 90, 91, 92, 93, 94, 95, 96, 97, 98, or 99% identity with a sequence as set forth in SEQ ID NO 3 and is at least 15, at least 18, at least 22, at least 25, or at least 30 nucleotides in length. In some embodiments, the forward primer has the sequence set forth in SEQ ID NO:3.

In some embodiments, the forward oligonucleotide primer comprises the sequence set forth in SEQ ID NO 4, or a sequence having at least or about 85, 90, 91, 92, 93, 94, 95, 96, 97, 98, or 99% identity with a sequence as set forth in SEQ ID NO 4. In some embodiments, the forward oligonucleotide primer comprises the sequence set forth in SEQ ID NO 4 and is at least 15, at least 18, at least 22, at least 25, or at least 30 nucleotides in length. In some embodiments, the forward oligonucleotide primer comprises a sequence having at least or about 85, 90, 91, 92, 93, 94, 95, 96, 97, 98, or 99% identity with a sequence as set forth in SEQ ID NO 4 and is at least 15, at least 18, at least 22, at least 25, or at least 30 nucleotides in length. In some embodiments, the forward primer has the sequence set forth in SEQ ID NO:4.

In some embodiments, the forward oligonucleotide primer comprises the sequence set forth in SEQ ID NO 5, or a sequence having at least or about 85, 90, 91, 92, 93, 94, 95, 96, 97, 98, or 99% identity with a sequence as set forth in SEQ ID NO 5. In some embodiments, the forward oligonucleotide primer comprises the sequence set forth in SEQ ID NO 5 and is at least 15, at least 18, at least 22, at least 25, or at least 30 nucleotides in length. In some embodiments, the forward oligonucleotide primer comprises a sequence having at least or about 85, 90, 91, 92, 93, 94, 95, 96, 97, 98, or 99% identity with a sequence as set forth in SEQ ID NO 5 and is at least 15, at least 18, at least 22, at least 25, or at least 30 nucleotides in length. In some embodiments, the forward primer has the sequence set forth in SEQ ID NO:5.

In some embodiments, the reverse oligonucleotide primer comprises the sequence set forth in SEQ ID NO 6, or a sequence having at least or about 85, 90, 91, 92, 93, 94, 95, 96, 97, 98, or 99% identity with a sequence as set forth in SEQ ID NO 6. In some embodiments, the reverse oligonucleotide primer comprises the sequence set forth in SEQ ID NO 6 and is at least 15, at least 18, at least 22, at least 25, or at least 30 nucleotides in length. In some embodiments, the reverse oligonucleotide primer comprises a sequence having at least or about 85, 90, 91, 92, 93, 94, 95, 96, 97, 98, or 99% identity with a sequence as set forth in SEQ ID NO 6 and is at least 15, at least 18, at least 22, at least 25, or at least 30 nucleotides in length. In some embodiments, the reverse primer has the sequence set forth in SEQ ID NO:6.

In some embodiments, the reverse oligonucleotide primer comprises the sequence set forth in SEQ ID NO 7, or a sequence having at least or about 85, 90, 91, 92, 93, 94, 95, 96, 97, 98, or 99% identity with a sequence as set forth in SEQ ID NO 7. In some embodiments, the reverse oligonucleotide primer comprises the sequence set forth in SEQ ID NO 7 and is at least 15, at least 18, at least 22, at least 25, or at least 30 nucleotides in length. In some embodiments, the reverse oligonucleotide primer comprises a sequence having at least or about 85, 90, 91, 92, 93, 94, 95, 96, 97, 98, or 99% identity with a sequence as set forth in SEQ ID NO 7 and is at least 15, at least 18, at least 22, at least 25, or at least 30 nucleotides in length. In some embodiments, the reverse primer has the sequence set forth in SEQ ID NO:7.

In some embodiments, (i) the forward oligonucleotide primer is as set forth in SEQ ID NO 3, or a sequence having at least or about 85, 90, 91, 92, 93, 94, 95, 96, 97, 98, or 99% identity with a sequence as set forth in SEQ ID NO 3 and (ii) the reverse oligonucleotide primer is as set forth in SEQ ID NO 6, or a sequence having at least or about 85, 90, 91, 92, 93, 94, 95, 96, 97, 98, or 99% identity with a sequence as set forth in SEQ ID NO 6. In some embodiments the forward oligonucleotide primer is as set forth in SEQ ID NO 3 and the reverse oligonucleotide primer is as set forth in SEQ ID NO 6.

In some embodiments, (i) the forward oligonucleotide primer is as set forth in SEQ ID NO 3, or a sequence having at least or about 85, 90, 91, 92, 93, 94, 95, 96, 97, 98, or 99% identity with a sequence as set forth in SEQ ID NO 3 and (ii) the reverse oligonucleotide primer is as set forth in SEQ ID NO 7, or a sequence having at least or about 85, 90, 91, 92, 93, 94, 95, 96, 97, 98, or 99% identity with a sequence as set forth in SEQ ID NO 7. In some embodiments the forward oligonucleotide primer is as set forth in SEQ ID NO 3 and the reverse oligonucleotide primer is as set forth in SEQ ID NO 7.

In some embodiments, (i) In some embodiments, the forward oligonucleotide primer is as set forth in SEQ ID NO 4, or a sequence having at least or about 85, 90, 91, 92, 93, 94, 95, 96, 97, 98, or 99% identity with a sequence as set forth in SEQ ID NO 4 and (ii) the reverse oligonucleotide primer is as set forth in SEQ ID NO 6, or a sequence having at least or about 85, 90, 91, 92, 93, 94, 95, 96, 97, 98, or 99% identity with a sequence as set forth in SEQ ID NO 6. In some embodiments the forward oligonucleotide primer is as set forth in SEQ ID NO 4 and the reverse oligonucleotide primer is as set forth in SEQ ID NO 6.

In some embodiments, (i) In some embodiments, the forward oligonucleotide primer is as set forth in SEQ ID NO 4, or a sequence having at least or about 85, 90, 91, 92, 93, 94, 95, 96, 97, 98, or 99% identity with a sequence as set forth in SEQ ID NO 4 and (ii) the reverse oligonucleotide primer is as set forth in SEQ ID NO 7, or a sequence having at least or about 85, 90, 91, 92, 93, 94, 95, 96, 97, 98, or 99% identity with a sequence as set forth in SEQ ID NO 7. In some embodiments the forward oligonucleotide primer is as set forth in SEQ ID NO 4 and the reverse oligonucleotide primer is as set forth in SEQ ID NO 7.

In some embodiments, the forward oligonucleotide primer is as set forth in SEQ ID NO 5, or a sequence having at least or about 85, 90, 91, 92, 93, 94, 95, 96, 97, 98, or 99% identity with a sequence as set forth in SEQ ID NO 5 and (ii) the reverse oligonucleotide primer is as set forth in SEQ ID NO 6, or a sequence having at least or about 85, 90, 91, 92, 93, 94, 95, 96, 97, 98, or 99% identity with a sequence as set forth in SEQ ID NO 6. In some embodiments the forward oligonucleotide primer is as set forth in SEQ ID NO 5 and the reverse oligonucleotide primer is as set forth in SEQ ID NO 6.

In some embodiments, the forward oligonucleotide primer is as set forth in SEQ ID NO 5, or a sequence having at least or about 85, 90, 91, 92, 93, 94, 95, 96, 97, 98, or 99% identity with a sequence as set forth in SEQ ID NO 5 and (ii) the reverse oligonucleotide primer is as set forth in SEQ ID NO 7, or a sequence having at least or about 85, 90, 91, 92, 93, 94, 95, 96, 97, 98, or 99% identity with a sequence as set forth in SEQ ID NO 7. In some embodiments the forward oligonucleotide primer is as set forth in SEQ ID NO 5 and the reverse oligonucleotide primer is as set forth in SEQ ID NO 7.

Also provided herein is an oligonucleotide probe for use in the detection of a reverse-transcriptase dependent amplicon. In some embodiments, the provided method including amplification methods such as is described in Section II.B.2. In some embodiments, these methods include systems of quantitative PCR using an oligonucleotide probe comprising a fluorophore. Here, excitation of the fluorophore results in the release of a fluorescent signal by the fluorophore which is quenched by the quencher. In some embodiments, amplicons can be detected by the 5′-3′ exonuclease activity of the TAQ DNA polymerase, which degrades double-stranded DNA encountered during extension of the PCR primer, thus releasing the fluorophore from the probe. Thereafter, the fluorescent signal is no longer quenched and accumulation of the fluorescent signal, which is directly correlated with the amount of target DNA, can be detected in real-time with an automated fluorometer.

In some embodiments, the oligonucleotide probe for detecting the reverse-transcriptase dependent amplicon is specific for the product obtained by carrying out PCR in the presence any of the provided forward and reverse primer sets as described. In some embodiments, the oligonucleotide probe is specific for (i.e. complementary to) a contiguous sequence within a third region that is between the first region and the second region of the SIN viral vector. In some embodiments, the oligonucleotide probe is specific for a contiguous sequence present within the viral LTR. In some embodiments, the oligonucleotide probe is specific for third region that includes a sequence present in the R and/or U5 region of the viral LTR. It is within the level of a skilled artisan to choose an appropriate probe sequence.

In some embodiments, the oligonucleotide probe has a length of at least 10 nucleotides. In some embodiments, the oligonucleotide probe is between 10-25, 10-20, 10-18, 10-15, 15-25, 15-22, 15-18, 18-25, 18-22, or 22-25 nucleotides in length. In some of any provided embodiments, the oligonucleotide probe has a sequence that is 10, 11, 12, 13, 14, 15, 16, 17, 18, 19, 20, 21, 22, 23, 24, 25, 26, 27, 28, 29, or 30 nucleotides in length.

In some embodiments, the oligonucleotide probe has (i) the sequence set forth in SEQ ID NO 8, (ii) a sequence having at least or about 85, 90, 91, 92, 93, 94, 95, 96, 97, 98, or 99% identity with a sequence as set forth in SEQ ID NO 8; or (ii) a sequence that is a complement of (i) or (ii). In some embodiments, the oligonucleotide probe has the sequence set forth in SEQ ID NO:8 or the complement thereof. In some embodiments, the oligonucleotide probe is set forth in SEQ ID NO:8.

In some embodiments, the oligonucleotide probe has (i) the sequence set forth in SEQ ID NO 9, (ii) a sequence having at least or about 85, 90, 91, 92, 93, 94, 95, 96, 97, 98, or 99% identity with a sequence as set forth in SEQ ID NO 9; or (ii) a sequence that is a complement of (i) or (ii). In some embodiments, the oligonucleotide probe has the sequence set forth in SEQ ID NO:9 or the complement thereof. In some embodiments, the oligonucleotide probe is set forth in SEQ ID NO:9.

Various methods and tools are available for choosing and designing primers and probes including, but not limited to Primer-BLAST, Primer3, Primer3Plus, PrimerQuest, OligoPerfect, PerlPrimer, OLIGO, AutoPrime, RExPrimer, and BatchPrimer3.

The provided probes for detecting the reverse-transcriptase dependent amplicon may be labeled with different detectable means. This detectable means refers to compounds, biomolecules or biomimetics that can be conjugated, connected, or attached to probes to provide quantitative indices such as density, concentration, quantity, etc. Examples of the detectable means include fluorescent markers, luminescents, bioluminescents, and radio isotopes, but are not limited thereto. If used together, two or more fluorescent markers may be different in color. Details and selection of the fluorescent markers are known to those skilled in the art (See e.g., Bao et al., Annual Review of Biomedical Engineering (11): 25-47, 2009).

In some embodiments, the oligonucleotide probe is labeled at the 5′ end with a fluorescent marker selected from the group consisting of FAM, VIC, TET, JOE, HEX, CY3, CY5, ROX, RED610, TEXAS RED, RED670, and NED. In some embodiments, the oligonucleotide probe is labeled at the 3′ end with a fluorescent marker selected from the group consisting of FAM, VIC, TET, JOE, HEX, CY3, CY5, ROX, RED610, TEXAS RED, RED670, and NED.

In some embodiments, the oligonucleotide probe is labeled at the 3′ end with a fluorescence quencher. In some embodiments, the quencher is part of a fluorescent probe comprising a fluorophore and is capable of accepting the energy emitted by the fluorophore, thereby quenching the emission of the fluorophore. A quencher can be fluorescent, which releases the accepted energy as light, or non-fluorescent, which releases the accepted energy as heat, and can be attached at any location along the length of the probe. In some embodiments, the oligonucleotide probe is labeled at the 5′ end with a fluorescence quencher. In some embodiments, the quencher is selected from the group consisting of 6-TAMRA, BHQ-1, 2, 3 and MGBNFQ.

In some embodiments, the oligonucleotide probe for detecting the reverse-transcriptase dependent amplicon (i.e., SEQ ID NOs 8-9), is specific for the product obtained by carrying out PCR in the presence of a primer set specific for the reverse-transcriptase dependent amplicon which comprises a forward primer and a reverse primer having nucleotides sequences of SEQ ID NOS: 3-5 or 6-7, respectively. In particular embodiments, the forward primer is set forth in SEQ ID NO:3, the reverse primer is set forth in SEQ ID NO:6 and the oligonucleotide probe is set forth in SEQ ID NO:8.

Oligonucleotide probes and primers provided herein can be synthesized by a number of methods. See, e.g., Ozaki et al., Nucleic Acids Research 20: 5205-5214 (1992); Agrawal et al., Nucleic Acids Research 18: 5419-5423 (1990). Among the methods are methods involving solid phase synthesis via phosphoramidite chemistry, such as disclosed in U.S. Pat. Nos. 4,458,066 and 4,415,732, incorporated herein by reference; Beaucage et al, Tetrahedron (1992) 48:2223-2311; and Applied Biosystems User Bulletin No. 13 (1 Apr. 1987). Other chemical synthesis methods include, for example, the phosphotriester method described by Narang et al, Meth. Enzymol. (1979) 68:90 and the phosphodiester method disclosed by Brown et al, Meth. Enzymol. (1979) 68: 109. Poly(A) or poly(C), or other non-complementary nucleotide extensions may be incorporated into oligonucleotides using these same methods. Hexaethylene oxide extensions may be coupled to the oligonucleotides by methods known in the art. Cload et al, J. Am. Chem. Soc. (1991) 113:6324-6326; U.S. Pat. No. 4,914,210 to Levenson et al; Durand et al, Nucleic Acids Res. (1990) 18:6353-6359; and Horn et al, Tet. Lett. (1986) 27:4705-4708. In an example, oligonucleotide probes can be synthesized on an automated DNA synthesizer such as the ABI 3900 DNA Synthesizer (Applied Biosystems, Foster City, CA). Alternative chemistries, e.g. resulting in non-natural backbone groups, such as phosphorothioate, phosphoramidate, and the like, may also be employed provided that the hybridization efficiencies of the resulting oligonucleotides are not adversely affected.

### 2. Amplification Methods

In some embodiments, determining and/or quantifying reverse-transcribed DNA can be performed using methods for determining and/or quantifying a particular sequence of reverse-transcribed DNA. In some aspects, methods used to detect or quantitate nucleic acid sequences, such as quantitative polymerase chain reaction (qPCR), can be used in detecting a particular sequence of reverse-transcribed DNA (i.e., a particular amplicon).

In some embodiments, detecting a viral nucleic acid can be accomplished using a probe or primer that specifically binds or recognizes all or a portion of the amplicon sequence. In some embodiments, the probe can specifically detect (bind or recognize) at least a portion of the amplicon. In some embodiments, the primers can specifically amplify at least a portion of the amplicon. In some embodiments, the primers and/or probe are specific for an amplicon that is reverse-transcriptase dependent.

In some embodiments, other methods of nucleic acid amplification known to a skilled artisan can be used to amplify or detect viral nucleic acid in a sample and/or quantify reverse-transcribed DNA in a sample. In some embodiments, methods for DNA amplification include isothermal methods. In some embodiments, the detecting of viral nucleic acid in a sample and/or quantifying reverse-transcribed DNA in a sample is completed by an isothermal method that includes but is not limited to LAMP, Nucleic Acid Sequence Based Amplification (NASBA), Strand Displacement Amplification (SDA), Multiple Displacement Amplification (MDA), Rolling Circle Amplification (RCA), Ligase Chain Reaction (LCR), Helicase Dependent Amplification (HDA), Ramification Amplification Method (RAM), Recombinase Polymerase Amplification (RPA), or any other method known in the art. For example, LAMP is a method which relies on the auto-cycling strand displacement DNA synthesis which is carried out in the presence of B. stearothermophylus (Bst) DNA polymerase, deoxyribonucleotide triphosphate (dNTPs), specific primers and the target DNA template. Further examples include the RPA reaction, in which recombinase enzymes form complexes with oligonucleotide primers and with the homologous sequences in duplex DNA. A single-stranded DNA binding (SSB) protein binds to the displaced DNA strand and DNA amplification by polymerase is then initiated from the primer.

In some embodiments, the detecting of viral nucleic acid in a sample and/or quantifying reverse-transcribed DNA in a sample is completed by quantitative polymerase chain reaction (qPCR; also known as real-time PCR). In some embodiments, qPCR enables detection of amplification product (i.e., amplicon) in real time, and after each cycle of amplification. In some embodiments, qPCR uses a reporter molecule, such as a fluorescent reporter molecule, in each reaction thereby yielding a direction relationship between the reporter and amplicon quantities. In some aspects, the amplicon specific primers and/or probes are fluorescently labeled. In some embodiments, the probe is a fluorescent probe and the measured fluorescence following each amplification cycle is proportional to the amount of amplicon. In some embodiments, change in fluorescent intensity over the repeated cycles is used to quantify amplicon produced in each cycle.

In some embodiments, the detecting of viral nucleic acid in a sample and/or quantifying reverse-transcribed DNA in a sample is completed by a combination of PCR and non-PCR isothermal methods. Such combinatorial methods include PCR-LAMP and PCDR (PCR-SDA). For example, PCR-LAMP combines a thermocycling mode at the initial stage of amplification with an isothermal LAMP amplification during the following stage.

In some embodiments, digital PCR amplification methods are carried out. Digital amplification (e.g., digital PCR or dPCR) is a highly sensitive quantitation method for nucleic acids. The method can detect and quantify nucleic acids by directly measuring the number of target molecules without relying on any normalization standard or external standards. In this manner, the absolute number of target molecules can be determined, with a lower limit being a single copy of the molecule.

In some embodiments, for digital amplification a sample can be first diluted and divided into thousands to tens of thousands of micro reaction chambers, so that most reaction chambers (e.g., at least 50%, 75%<90%, 95 or 99%) contains only either zero or one copy of the target sequence (a small number of reaction chambers may contain multiple copies). By counting the number of reaction chambers with positive amplification results, the absolute number of target molecules in the original sample can be determined.

In some embodiments, the distribution of the target molecules across the partitions can be seen as a Poisson process (the targets end up in partitions independently and with a fixed rate). Poisson statistics thus allow a more accurate calculation of the initial number of targets from the number of positive and negative partitions taking into account that some reaction chambers receive multiple copies.

In some aspects, in comparison to traditional PCR technology, dPCR or other digital amplification method is considered to have multiple advantages, including low required sample amount, reduced consumption of reagents, absolute quantification of nucleic acid molecules, reduced interference among different copies within a sample, and superb sensitivity and specificity. Furthermore, the standard division process of the reaction system in digital amplification can greatly reduce the concentration of background sequences that could compete with the target sequence. In some aspects, dPCR is generally linear and are sensitive, capable of detecting or quantifying very small amounts of DNA. In some aspects, dPCR permits absolute quantification of a DNA sample using a single molecule counting method without a standard curve, and absolute quantification can be obtained from PCR for a single partition per well (see Pohl et al., (2004) Expert Rev. Mol. Diagn. 4(1), 41-47).

In some aspects, dPCR methods can be used to generate a plurality of partitions, such as thousands of partitions, to carry out a plurality of individual PCR reactions in parallel. In some aspects, a sample is partitioned so that individual nucleic acid molecules within the sample are localized and concentrated within many separate regions. Micro well plates, capillaries, micro- or nanofluidics, oil emulsion, emulsion chemistry and/or arrays of miniaturized chambers with nucleic acid binding surfaces can be used to partition the samples. Exemplary compositions for carrying out dPCR can include template nucleic acid (e.g., isolated DNA from cells known or suspected of containing a reverse transcribed viral nucleic acid), fluorescence-quencher probes, primers, and a PCR master mix, which contains DNA polymerase, dNTPs, MgCl₂, and reaction buffers at optimal concentrations. The PCR solution is divided into smaller reactions and are then made to run PCR individually. The partitioning of the sample allows one to estimate the number of different molecules by assuming that the molecule population follows the Poisson distribution, thus accounting for the possibility of multiple target molecules inhabiting a single partition.

In some aspects, dPCR involves analyzing the results by a digital method (because the resultant signal has a binary value: “0” or “1”). In some aspects, dPCR can be used to analyze a large volume, analyze various samples at the same time, and multiple assessments can be performed at the same time. In some aspects, for digital PCR, each partition comprising a sample sequence template (e.g., DNA from a cell suspected of containing a reverse transcribed viral nucleic acid) prepared so as to be diluted to an average copy number of the sequence is 0.5-1. The dilution is important to obtain reliable results for quantification, to signals that appear in a Poisson distribution. In each well, amplification primers specific for the target sequence (e.g., reverse-transcriptase dependent amplicon) and a fluorescent probe, is dispensed and emulsion PCR is performed. In some aspects, a well exhibiting a fluorescent signal is counted as a value of “1”, because a sample having a sequence copy number of 1 is dispensed into the well and shows the signal after amplification, and a well showing no signal is counted as “0”, because a sample not containing a copy of the sequence is dispensed into the well and shows no signal due to no amplification. Using Poisson's law of small numbers, the distribution of target molecule within the sample can be accurately approximated, permitting an absolute quantification of the target sequences in the PCR product.

Exemplary commercially available apparatuses or systems for dPCR include Raindrop™ Digital PCR System (Raindance™ Technologies); QX200™ Droplet Digital™ PCR System (Bio-Rad); BioMark™ HD System and qdPCR 37K™ IFC (Fluidigm Corporation) and QuantStudio™ 3D Digital PCR System (Life Technologies™) (see, e.g., Huggett et al. (2013) Clinical Chemistry 59: 1691-1693; Shuga, et al. (2013) Nucleic Acids Research 41(16): e159; Whale et al. (2013) PLoS One 3: e58177).

In some embodiments, the dPCR can be a microfluidic-chip-based dPCR. In some embodiments, the dPCR is a Droplet-based dPCR. Depending on the application a skilled artisan can choose an appropriate dPCR platform. For example, a microfluidic-chip-based dPCR can have up to several hundred partitions per panel. Droplet-based dPCR usually has approximately 20,000 partitioned droplets, but it can have up to 10,000,000.

In some embodiments, the detecting of viral nucleic acid in a sample and/or quantifying reverse-transcribed DNA in a sample is completed by digital droplet polymerase chain reaction (ddPCR). ddPCR is a type of qPCR wherein the entire PCR solution, containing at least the sample, primers, and probes, is separated into a plurality of reaction droplets through water-oil emulsion chemistry, to generate numerous droplets. In some embodiments, droplets are generated through the use of surfactants (see, e.g., Hindson et al., (2011) Anal Chem 83(22): 8604-8610; Pinheiro et al., (2012) Anal Chem 84, 1003-1011). In some aspects, the PCR sample is partitioned into nanoliter-size samples and encapsulated into oil droplets. In some embodiments, droplets are generated via a droplet generator which applies vacuum pressure to the reaction solution in wells. In an exemplary case, approximately 20,000 oil droplets for individual reactions can be made from a 20 μL sample volume.

In some embodiments, each droplet is subsequently run as an individual reaction. In some aspects, following PCR, each droplet is analyzed or read to determine the fraction of PCR-positive droplets (e.g., binary “0” or “1” assigned in each droplet based on the detectable signal, such as fluorescence signal) in the original sample. Data from the droplet distribution is analyzed via Poisson distribution, wherein the copies of target amplicon per droplet (CPD) can be mathematically determined using the fraction of “1” fluorescent droplets (p), using the equation CPD=ln(1−p). In some embodiments, ddPCR can be used for absolute quantification of the target amplicon.

Exemplary commercially available apparatuses or systems for ddPCR include QX100 and QX200 from Bio-Rad®

### C. Determining Vector Copy Number

In some embodiments, the provided methods are used to quantify vector copy number as present in a sample. In some embodiments, the sample contains or is derived from a cell that has been transduced with a SIN viral vector. In some embodiments, the cell has been transduced with a retroviral vector. In some embodiments, the cell has been transduced with a gammaretroviral vector. In particular embodiments, the cell has been transduced with a lentiviral vector.

Provided herein are methods of detecting and quantifying vector copy number in a sample, such methods may include assessing the presence of a reverse-transcriptase-dependent amplicon after the PCR. In some embodiments, the SIN viral vector also contains non-viral nucleic acid, such as a nucleic acid sequence encoding an exogenous agent, such as a recombinant protein. In some embodiments, ability to detect or quantify vector copy number based on the reverse-transcriptase-dependent amplicon is indicative of the integration of, and copy number of, the nucleic acid encoding the exogenous agent in the genome of the cell.

In some embodiments, the reverse-transcriptase-dependent amplicon is only present after reverse-transcription. In some embodiments, the reverse-transcriptase-dependent amplicon is not present on residual and/or bystander molecules (i.e., plasmids including production plasmids, free nucleotides, or other secondary structures which may result from cellular transduction). In some embodiments, the reverse-transcriptase-dependent amplicon is present following integration of viral (and also transgene) nucleic acids present in the SIN viral vector. In some embodiments, the reverse-transcriptase-dependent amplicon, i.e., target amplicon, comprises the sequence set forth in SEQ ID NO: 10, or a sequence having at least or about 85, 90, 91, 92, 93, 94, 95, 96, 97, 98, or 99% identity with SEQ ID NO:10.

In some embodiments, the detecting and quantifying comprises determining and/or quantifying reverse-transcribed DNA, such as determining the copy number of the reverse-dependent amplicon sequences, in one or more cells or in a sample, wherein the one or more cells has been transduced or is suspected of comprising a viral DNA. In some embodiments, the determining of the copy number of the reverse-dependent amplicon sequences can be performed in a portion of a population of cells, and can be normalized, averaged, and/or extrapolated to determine the copy number in the entire sample or entire population of cells. In some embodiments, the amount of the reverse-dependent amplicon sequence can include the concentration or copy number of the reverse-dependent amplicon sequence. In some embodiments, the concentration or copy number is an average concentration or copy number. In some embodiments, the concentration or copy number is an average concentration or copy number per a unit, such as per cell, per diploid genome, per volume, per mass or equivalent thereof, or otherwise normalized, extrapolated or averaged to be per a unit.

In some embodiments, detecting and quantifying of the reverse-dependent amplicon sequence comprises determining the concentration or copy number of the reverse-dependent amplicon sequence per diploid genome or per cell in the one or more cells. In some embodiments, the one or more cell comprises a population of cells in which a plurality of cells of the population comprise the reverse-dependent amplicon sequence. In some of any of the provided embodiments, the one or more cell comprises a population of cells in which a plurality of cells of the population is suspected of being transduced, i.e., comprising the reverse-dependent amplicon sequence. In some embodiments, the copy number is an average or mean copy number per diploid genome or per cell among the population of cells.

In some embodiments, determining the copy number comprises determining the number of copies of the reverse-dependent amplicon sequences present in one or more cells. In some embodiments, the copy number can be expressed as an average or mean copy number. In some embodiments, the copy number of a particular integrated reverse-dependent amplicon includes the number of integrants (containing reverse-dependent amplicon sequences) per cell. In some aspects, the copy number of a particular integrated reverse-dependent amplicon includes the number of integrants (containing reverse-dependent amplicon sequences) per diploid genome. In some aspects, the copy number of reverse-dependent amplicon sequence is expressed as the number of integrated reverse-dependent amplicon sequences per cell. In some aspects, the copy number of reverse-dependent amplicon sequence is expressed as the number of integrated reverse-dependent amplicon sequences per a particular type of cell, e.g., per cell expressing a particular phenotypic marker, or optionally per cell that expresses or produces an exogenous agent encoded by the introduced viral nucleic acid.

In some embodiments, in addition to quantification of the target amplicon sequence the level of a reference sequence, i.e. reference amplicon, is also assessed in order to confirm the validity, including sensitivity and/or specificity, of the assay. In some aspects, assessment of the amplicon of the reference gene is separate from the target amplicon, i.e. reverse-transcriptase-dependent amplicon. In some aspects, assessment of the amplicon of the reference gene is multiplexed with the target amplicon. In some instances, assessment of the amplicon of the reference gene controls for DNA quality and/or presence in the assay. In some embodiments, presence of the reference amplicon in a reaction, e.g., well or tube or droplet, confirms that DNA is present. In some embodiments, presence of the reference amplicon in a reaction, e.g., well or tube or droplet, of sufficient quality to be capable of undergoing PCR amplification, such as in digital droplet PCR. In some embodiments, the reference amplicon is known to be present in the genome of the cells in one or two copies and thus can be used to normalize the target amplicon.

In some embodiments, the determining the copy number of the reverse-dependent amplicon sequence comprises assessing the concentration or copy number of the reverse-dependent amplicon sequence and normalizing the concentration or copy number to the concentration or copy number of a reference gene. In other cases, the determining the copy number of the reverse-dependent amplicon sequence comprises assessing the concentration or copy number of the reverse-dependent amplicon sequence and normalizing the concentration or copy number to the concentration or copy number of a standard curve. For instance, a standard curve may be based on samples containing a known concentration or copy number of reverse-dependent amplicon sequences.

In some embodiments, the copy number is expressed as a normalized value. In some embodiments, the copy number is quantified as a number of copies of the reverse-dependent amplicon sequence per genome or per cell. In some aspects, the per genome value is expressed as copy of the reverse-dependent amplicon sequence per diploid genome, as a typical human somatic cell, such as a T cell, contains a diploid genome. In some embodiments, the copy number can be normalized against the copy number of a known reference gene in the genome of the cell, such as determined by PCR amplification of a reference amplicon of the gene. In some embodiments, the reference gene is an endogenous gene known to be present in the genome of the cells in the sample. In some embodiments, the reference gene is known to be present in one or two copies in a diploid genome.

In some embodiments, the reference gene is a housekeeping gene. In some embodiments, the reference gene and/or housekeeping gene is RRP30 (encoding ribonuclease P protein subunit p30), 18S rRNA (18S ribosomal RNA), 28S rRNA (28S ribosomal RNA), TUBA (α-tubulin), ACTB (β-actin), 02M (β2-microglobulin), ALB (albumin), RPL32 (ribosomal protein L32), TBP (TATA sequence binding protein), CYCC (cyclophilin C), EF1A (elongation factor 1α), GAPDH (glyceraldehyde-3-phosphate dehydrogenase), HPRT (hypoxanthine phosphoribosyl transferase), RPII (RNA polymerase II) or human telomerase reverse transcriptase (hTERT). In some embodiments, the reference gene is hTERT.

In some embodiments, the copy number is quantified as copy of the reverse-dependent amplicon sequence per microgram of DNA.

In some embodiments, the copy number is an average, mean, mode, or median copy number from a plurality or population of cells, such as a plurality or population of transduced cells or cells suspected of comprising a viral DNA. In some embodiments, the average or mean copy number is determined from a plurality or population of cells, such as a plurality or population of cells undergoing one or more steps of the transduction process. In some embodiments, a normalized average copy number is determined. For example, an average or mean copy number of the reverse-dependent amplicon sequences normalized to a reference or housekeeping gene, such as a known gene that is present in two copies in a diploid genome (e.g. hTERT). In some embodiments, normalization to a reference or housekeeping gene, can correspond to the copy number in a cell, such as a diploid cell. Thus, in some aspects, the normalized average or mean copy number can correspond to the average or mean copy number of the detected reverse-dependent amplicon sequences among a plurality and/or a population of cells.

In some embodiments, the determining vector copy number is carried out by polymerase chain reaction (PCR). In some embodiments, the PCR is quantitative polymerase chain reaction (qPCR) or droplet digital PCR, such as any described above. In some embodiments, the vector copy number and/or copy number of the reverse-dependent amplicon sequence is determined by droplet digital PCR. In some embodiments, the PCR is carried out using at least one primer that is complementary to at least a portion of the reverse-dependent amplicon sequence, and in some cases, at least one primer that is complementary to at least a portion of a reference or housekeeping gene.

### III. Viral Vector Particles

The provided methods can be carried out on cells that have been transduced with a SIN viral vector. In some of any of the provided embodiments, the viral vector is replication defective. In some of any of the provided embodiments, the viral vector is a retrovirus, or a lentivirus (i.e., HIV-vectors). In some embodiments, the viral vector contains nucleic acid for a transgene sequence encoding an exogenous agent.

Biological methods for introducing an exogenous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the viral particles bilayer of amphipathic lipids is or comprises lipids derived from an infected host cell. In some embodiments, the lipid bilayer is a viral envelope. In some embodiments, the viral particles envelope is obtained from a host cell. In some embodiments, the viral particles envelope is obtained by the viral capsid from the source cell plasma membrane. In some embodiments, the lipid bilayer is obtained from a membrane other than the plasma membrane of a host cell. In some embodiments, the viral particles envelope lipid bilayer is embedded with viral proteins, including viral glycoproteins. The viral envelope may comprise a fusogen, that is endogenous to the virus or is a pseudotyped fusogen.

In some embodiments, the viral vector's lumen or cavity comprises a viral nucleic acid, e.g., a retroviral nucleic acid, e.g., a lentiviral nucleic acid. The viral nucleic acid may be a viral genome. In some embodiments, the viral vector may further comprises one or more viral non-structural proteins, e.g., in its cavity or lumen.

In some aspects, the viral vector particle is limited in the number of polynucleotides that can be packaged. In some embodiments, nucleotides encoding polypeptides to be packaged can be modified such that they retain functional activity with fewer nucleotides in the coding region than that which encodes for the wild-type peptide. Such modifications can include truncations, or other deletions. In some embodiments, more than one polypeptide can be expressed from the same promoter, such that they are fusion polypeptides. In some embodiments, the insert size to be packaged (i.e., viral genome, or portions thereof; or heterologous polynucleotides as described) can be between 500-1000, 1000-2000, 2000-3000, 3000-4000, 4000-5000, 5000-6000, 6000-7000, or 7000-8000 nucleotides in length. In some embodiments, the insert can be over 8000 nucleotides, such as 9000, 10,000, or 11,000 nucleotides in length.

In some embodiments, the viral vector particle packages nucleic acids from host cells carrying one or more viral nucleic acids (e.g. retroviral nucleic acids) during the expression process. In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

In some embodiments, the nucleic acids do not encode any genes involved in virus replication. In particular embodiments, the viral vector particle, e.g. retrovirus particle such as a lentivirus particle, that is replication defective.

In some aspects, provided herein is a replication incompetent (also referred to herein as replication defective) viral vector particle, that cannot participate in replication in the absence of the packaging cell (i.e., viral vector particles are not produced from the transduced cell). In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, because the right (3′) LTR U3 region can be used as a template for the left (5′) LTR U3 region during viral replication and, therefore, absence of the U3 enhancer-promoter inhibits viral replication. In embodiments, the 3′ LTR is modified such that the U5 region is removed, altered, or replaced, for example, with an exogenous poly(A) sequence. The 3′ LTR, the 5′ LTR, or both 3′ and 5′ LTRs, may be modified LTRs. Other modifications to the viral vector, i.e., retroviral or lentiviral vector, to render said vector replication incompetent are known in the art. For example, in a replication-defective retroviral vector genome gag, pol and env may be absent or not functional.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, the viral vector particle, such as retroviral vector particle, further comprises one or more of gag polyprotein, polymerase (e.g., pol), integrase (e.g., a functional or non-functional variant), protease, and a fusogen. In some embodiments, the lipid particle further comprises rev. In some embodiments, one or more of the aforesaid proteins are encoded in the retroviral genome (i.e., the insert as described above), and in some embodiments, one or more of the aforesaid proteins are provided in trans, e.g., by a helper cell, helper virus, or helper plasmid.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein. The vector may be configured as a split-intron vector, e.g., as described in PCT patent application WO 99/15683, which is herein incorporated by reference in its entirety.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

A viral vector can comprise a nucleic acid molecule (e.g., a transfer plasmid) that includes virus-derived nucleic acid elements that typically facilitate transfer of a nucleic acid molecule (e.g. including nucleic acid encoding an exogenous agent) or integration into the genome of a cell or to a viral particle that mediates nucleic acid transfer. Viral vector particles will typically include various viral components and sometimes also host cell components in addition to nucleic acid(s). A viral vector can comprise a virus or viral particle capable of transferring a nucleic acid into a cell (e.g. nucleic acid encoding an exogenous agent), or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′)R-U5-one or more first nucleotide sequences-U3-R(3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or in combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used or combined, and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene and can be inserted as one or multiple copies.

In some embodiments, expression of heterologous sequences (e.g. nucleic acid encoding an exogenous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE.

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone polyA sequence (BGHpA), a rabbit b-globin polyA sequence (rPgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi packaging signal, RRE), and/or other elements that increase exogenous gene expression (e.g., poly (A) sequences), and may optionally comprise a WPRE or HPRE.

### A. Methods of Generating Viral Vector Particles

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, viral vector particles may be produced in multiple cell culture systems including bacteria, mammalian cell lines, insect cell lines, yeast and plant cells. Exemplary methods for producing viral vector particles are described.

In some embodiments, elements for the production of a viral vector, i.e., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line that is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles (i.e., a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments, expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

Typically, modern retroviral vector systems include viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

Generally, for viral vector particles as described in Section III, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The VLP could be used to deliver therapeutic RNA, in which case functional integrase and/or reverse transcriptase is not required. These VLPs could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U 1 small nuclear ribonucleoprotein particle, a Nova protein, a TF111 A protein, a TIS 11 protein, a trp RNA-binding attenuation protein (TRAP) or a pseudouridine synthase.

In some embodiments, formation of viral vector particles as described above in Section III can be detected by any suitable technique known in the art. Examples of such techniques include, e.g., electron microscopy, dynamic light scattering, selective chromatographic separation and/or density gradient centrifugation.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

### B. Pseudotyped Vector

Provided herein are viral vector particles, including targeting viral vector particles. In some aspects, such viral vector particles contain viral nucleic acid, such as retroviral nucleic acid, for example lentiviral nucleic acid. In particular embodiments, any provided targeted viral vector particles, is replication defective. In some embodiments, the viral vector particle is a lentiviral vector, in which the lentiviral vector is pseudotyped. In some embodiments, the viral vector particle, such as a lentiviral vector, is pseudotyped with a Paramyxovirus (e.g., henipavirus or morbillivirus) protein.

In some embodiments, the viral vector (i.e., SIN viral vector) further comprises a vector-surface targeting moiety which specifically binds to a target ligand. In some embodiments, the vector-surface targeting moiety is a polypeptide. In some embodiments, the polypeptide is a fusogen.

In some embodiments, the provided viral vector contains one or more fusogens. In some embodiments, the viral vector contains an exogenous or overexpressed fusogen. In some embodiments, the fusogen is disposed in the lipid bilayer. In some embodiments, the fusogen facilitates the fusion of the lipid particle to a membrane. In some embodiments, the membrane is a plasma cell membrane. In some embodiments, the viral vector comprising the fusogen integrates into the membrane into a lipid bilayer of a target cell. In some embodiments, the fusogen results in mixing between lipids in the lipid particle and lipids in the target cell. In some embodiments, the fusogen results in formation of one or more pores between the interior of the non-cell particle and the cytosol of the target cell.

In some embodiments, fusogens are protein based, lipid based, and chemical based fusogens. In some embodiments, the viral vector contains a first fusogen that is a protein fusogen and a second fusogen that is a lipid fusogen or chemical fusogen. In some embodiments, the fusogen binds a fusogen binding partner on a target cell surface. In some embodiments, the viral vector is pseudotyped with the fusogen. In some embodiments, retroviral envelope proteins, e.g. lentiviral envelope proteins, are pseudotyped with a fusogen.

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), Baboon endogenous retrovirus envelope glycoprotein (BaEV), and Borna disease virus (BDV) glycoprotein (BDV G).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a canine distemper virus F protein, a tupaia paramyxovirus F protein, a paramyxovirus F protein, a Hendra virus F protein, a Henipavirus F protein, a Morbillivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein or a biologically active portion thereof.

In some embodiments, the vector-surface targeting moiety comprises a protein with a hydrophobic fusion peptide domain. In some embodiments, the vector-surface targeting moiety comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein or a bat Paramyxovirus F protein or a biologically active portion thereof.

In particular embodiments, the F protein or the functionally active variant or biologically active portion thereof retains fusogenic activity in conjunction with a Henipavirus G protein, such as a G protein set forth above (e.g. NiV-G or HeV-G). Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L

In particular embodiments, the G protein or functionally active variant or biologically active portion is a protein that retains fusogenic activity in conjunction with a Henipavirus F protein, e.g. NiV-F or HeV-F. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

It will further be recognized by those skilled in the art that many viruses such as paramyxoviruses bind to sialic acid receptors, and hence the corresponding derivative vehicles can deliver their contents generically to nearly any kind of cell that expresses sialic acid receptors. Other viruses such as Nipah virus (NiV) and HIV bind to protein receptors, and hence the corresponding vehicles have a specificity that matches the natural tropisms for each virus and its surface proteins.

Furthermore, it will be recognized that technology exists to “re-target” attachment proteins, making it so that the viral vector only interact with particular cells or cell types that express a marker protein of interest (Msaouel et al., Meths Mol Biol 797: 141-162, 2012). Thus, viral vector surface glycoproteins proteins can be supplemented with or replaced by other targeting proteins, including but not necessarily limited to antibodies and antigen binding fragments thereof, receptor ligands, and other approaches that will be apparent to those skilled in the art given the benefit of the present disclosure. In some embodiments, the vector-surface targeting moiety is a polypeptide. In some embodiments, the polypeptide is a fusogen.

In some embodiments, the vector-surface targeting moiety binds a target ligand. In some embodiments, the target ligand can be expressed in an organ or cell type of interest. In particular embodiments, the fusogen (e.g. a G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred.

In some embodiments, protein fusogens may be re-targeted by covalently conjugating a targeting-moiety to the fusion protein. In some embodiments, the fusogen and targeting moiety are covalently conjugated by expression of a chimeric protein comprising the fusogen linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein.

In some embodiments, vector-surface targeting moiety is a peptide. In some embodiments, vector-surface targeting moiety is an antibody, such as a single domain antibody. In some embodiments, the antibody can be human or humanized. In some embodiments, antibody or portion thereof is naturally occurring. In some embodiments, the antibody or portion thereof is synthetic.

In some embodiments, the C-terminus of the vector-surface targeting moiety is attached to the C-terminus of the G protein (e.g., fusogen) or biologically active portion thereof. In some embodiments, the N-terminus of the vector-surface targeting moiety is exposed on the exterior surface of the lipid bilayer. In some embodiments, the N-terminus of the vector-surface targeting moiety binds to a cell surface molecule of a target cell. In some embodiments, the vector-surface targeting moiety specifically binds to a cell surface molecule present on a target cell. In some embodiments, the vector-surface targeting moiety is a protein, glycan, lipid or low molecular weight molecule.

### C. Exogenous Agent

In some embodiments, the viral vector particle comprising same described herein contains an exogenous agent. In some embodiments, the viral vector particle comprising same described herein contains a nucleic acid that encodes an exogenous agent. In some embodiments, the viral vector particle contains the exogenous agent. In some embodiments, the lipid particle contains a nucleic acid that encodes an exogenous agent. Reference to the coding sequence of the nucleic acid encoding the exogenous agent also is referred to herein as a payload gene. In some embodiments, the exogenous agent or the nucleic acid encoding the exogenous agent are present in the lumen of the viral vector particle.

In particular embodiments, an exogenous agent, such as a polynucleotide or polypeptide, is encapsulated within the lumen of a viral vector particles. Embodiments of provided viral vector particles may have various properties that facilitate delivery of a payload, such as, e.g., a desired transgene or exogenous agent, to a target cell. The exogenous agent may be a polynucleotide or a polypeptide. In some embodiments, a viral vector particles provided herein is administered to a subject, e.g., a mammal, e.g., a human. In such embodiments, the subject may be at risk of, may have a symptom of, or may be diagnosed with or identified as having, a particular disease or condition. In one embodiment, the subject has cancer. In some embodiments, the subject has a metabolic disorder. In one embodiment, the subject has an infectious disease. In some embodiments, the lipid particle contains nucleic acid sequences (polynucleotide) encoding an exogenous agent or a polypeptide exogenous agent for treating the disease or condition.

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor (e.g., a chimeric receptor or CAR), or an antibody; a nucleic acid, e.g., DNA, a chromosome (e.g. a human artificial chromosome), RNA, mRNA, siRNA, or miRNA; or a small molecule.

In some embodiments, the exogenous agent is not expressed naturally in the cell from which the lipid particle is derived. In some embodiments, the exogenous agent is expressed naturally in the cell from which the lipid particle is derived. In some embodiments, the exogenous agent is loaded into the lipid particle via expression in the cell from which the lipid particle is derived (e.g. expression from DNA or mRNA introduced via transfection, transduction, or electroporation). In some embodiments, the exogenous is expressed from DNA integrated into the genome or maintained episomally. In some embodiments, expression of the exogenous agent is constitutive. In some embodiments, expression of the exogenous agent is induced. In some embodiments, expression of the exogenous agent is induced immediately prior to generating the lipid particle. In some embodiments, expression of the exogenous agent is induced at the same time as expression of the fusogen.

In some embodiments, the exogenous agent may include one or more nucleic acid sequences, one or more polypeptides, a combination of nucleic acid sequences and/or polypeptides, and any combination thereof.

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells

In some embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In some embodiments, a viral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target.

In some embodiments, the lipid particle contains a nucleic acid that encodes a protein exogenous agent (also referred to as a “payload gene encoding an exogenous agent.”). In some embodiments, a lipid particle described herein comprises an exogenous agent which is or comprises a protein. In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), CRISPR-Cas proteins and homologs thereof), recombinases, and any combination thereof. In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

In some embodiments, a payload gene described herein encodes a chimeric antigen receptor (CAR) comprising an antigen binding domain. In some embodiments, an exogenous agent described herein comprises a chimeric antigen receptor (CAR) comprising an antigen binding domain. In some embodiments, the payload is or comprises a chimeric antigen receptor (CAR) comprising an antigen binding domain. In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, the exogenous agent is a nuclease for use in gene editing methods. In some embodiments, the nuclease is a zinc-finger nuclease (ZFN), transcription-activator like effector nuclease (TALEN), or a CRISPR-associated protein-nuclease (Cas). In some embodiments, the Cas is Cas9 from Streptococcus pyogenes. In some embodiments, the Cas is a Cas12a (also known as Cpf1) from a Prevotella or Francisella bacteria, or the Cas is a Cas12b from a Bacillus, optionally Bacillus hisashii. In some embodiments, the nuclease is MAD7 or CasX. In some embodiments, delivery of the nuclease is by a provided vector encoding the nuclease (e.g. Cas).

In some embodiments, the provided viral vector particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided viral vector particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the viral vector particle (e.g. lentiviral vector particle). For instance, a chimeric Cas9-protein fusion with the structural GAG protein can be packaged inside a lentiviral vector particle. In some embodiments, the fusion protein is a cleavable fusion protein between (i) a viral structural protein (e.g. GAG) and (ii) a nuclease protein (e.g. Cas protein, such as Cas 9).

In some embodiments, the exogenous agent includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

### IV. Kits and Articles of Manufacture

Provided herein are kits and articles of manufacture, such as those containing reagents for performing the methods herein (i.e., detecting a viral nucleic acid, quantifying reverse-transcribed DNA, and/or determining viral vector copy number in a sample).

In some embodiments, the kit can contain consumables in addition to reagents required for isolating DNA from the sample, such as reagents and consumables for PCR. In some embodiments, the provided kits comprise one or more probes and at least two primers (i.e., a primer pair). In some embodiments, the primer pair comprises a reverse primer and forward primer specific for all or a portion of the target amplicon. In some embodiments, the probe and/or primer can specifically bind (i.e., recognize) at least a portion of the target amplicon.

Also provided herein are primer pairs containing a reverse primer and a forward primer, such as for use in amplification of a reverse-transcriptase-dependent target amplicon. In some embodiments, the forward oligonucleotide primer is complementary to a contiguous sequence of nucleotides of a first region present within the deleted U3 (delU3) of the SIN viral vector, and the reverse oligonucleotide primer is complementary to a contiguous sequence of nucleotides of a second region downstream of the first region to make a replicable reverse-transcriptase-dependent amplicon. In some embodiments, the reverse-transcriptase-dependent amplicon is 100 base pairs to 500 base pairs in size. In some embodiments, the reverse-transcriptase-dependent amplicon is 200 base pairs to 400 base pairs in size.

In some embodiments, the first region comprises the U3 attachment sequence. In some embodiments, the first region (i.e., plus strand) comprises the sequence set forth in any one of SEQ ID NOS: 46-48. In some embodiments, the forward primer comprises at least a contiguous portion of any one of SEQ ID NOS: 46-48. In some embodiments, the first region comprises the sequence set forth in any one of SEQ ID NOS: 1 and 14-28 or a sequence having at least or about 85, 90, 91, 92, 93, 94, 95, 96, 97, 98, or 99% sequence identity to any one of SEQ ID NOS: 1 and 14-28. In some embodiments, the first region is set forth in SEQ ID NO:1.

In some embodiments, the second region comprises at least a portion of the primer binding site (PBS), the IFN-stimulated response element (ISRE), and/or the Psi (Ψ) packaging sequence of the SIN viral vector. In some embodiments, the second region (i.e., on the minus strand) comprises the sequence set forth in any one of SEQ ID NOS: 2 and 29-45 or a sequence having at least or about 85, 90, 91, 92, 93, 94, 95, 96, 97, 98, or 99% sequence identity to any one of SEQ ID NOS: 2 and 29-45.

In some embodiments, the forward primer is a contiguous sequence of nucleotides within a first region having the sequence set forth in SEQ ID NO:1 or a sequence having at least or about 85, 90, 91, 92, 93, 94, 95, 96, 97, 98, or 99% sequence identity thereto, and the reverse primer is a contiguous sequence of nucleotides within a second region having the sequence set forth in SEQ ID NO:2 or a sequence having at least or about 85, 90, 91, 92, 93, 94, 95, 96, 97, 98, or 99% sequence identity thereto. In some embodiments, the forward primer is a contiguous sequence of nucleotides within a first region (i.e., plus strand) having the sequence set forth in SEQ ID NO:1 and the reverse primer is complementary to a contiguous sequence of nucleotides within a second region (i.e., minus strand) having the sequence set forth in SEQ ID NO:2.

In some embodiments, the forward primer is a contiguous sequence of nucleotides within a first region having the sequence set forth in SEQ ID NO:14 r a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:29 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:14 and the reverse primer is a contiguous sequence of nucleotides within a second region having the sequence set forth in SEQ ID NO:29.

In some embodiments, the forward primer a contiguous sequence of nucleotides within a first region having the sequence set forth in SEQ ID NO:15 or a sequence having at least or about 85, 90, 91, 92, 93, 94, 95, 96, 97, 98, or 99% sequence identity thereto. and the reverse primer is a contiguous sequence of nucleotides within a second region having the sequence set forth in SEQ ID NO:30 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:15 and the reverse primer is a contiguous sequence of nucleotides within a second region having the sequence set forth in SEQ ID NO:30.

In some embodiments, the forward primer a contiguous sequence of nucleotides within a first region having the sequence set forth in SEQ ID NO:16 or a sequence having at least or about 85, 90, 91, 92, 93, 94, 95, 96, 97, 98, or 99% sequence identity thereto. and the reverse primer is a contiguous sequence of nucleotides within a second region having the sequence set forth in SEQ ID NO:31 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:16 and the reverse primer is a contiguous sequence of nucleotides within a second region having the sequence set forth in SEQ ID NO:31.

In some embodiments, the forward primer a contiguous sequence of nucleotides within a first region having the sequence set forth in SEQ ID NO:17 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:32 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:17 and the reverse primer is a contiguous sequence of nucleotides within a second region having the sequence set forth in SEQ ID NO:32.

In some embodiments, the forward primer is a contiguous sequence of nucleotides within a first region having the sequence set forth in SEQ ID NO:18 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:33 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:18 and the reverse primer is a contiguous sequence of nucleotides within a second region having the sequence set forth in SEQ ID NO:33.

In some embodiments, the forward primer is a contiguous sequence of nucleotides within a first region having the sequence set forth in SEQ ID NO:19 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:35 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:19 and the reverse primer is a contiguous sequence of nucleotides within a second region having the sequence set forth in SEQ ID NO:35.

In some embodiments, the forward primer is a contiguous sequence of nucleotides within a first region having the sequence set forth in SEQ ID NO:20 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:36 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:20 and the reverse primer is a contiguous sequence of nucleotides within a second region having the sequence set forth in SEQ ID NO:36.

In some embodiments, the forward primer is a contiguous sequence of nucleotides within a first region having the sequence set forth in SEQ ID NO:21 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:38 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:21 and the reverse primer is a contiguous sequence of nucleotides within a second region having the sequence set forth in SEQ ID NO:38.

In some embodiments, the forward primer is a contiguous sequence of nucleotides within a first region having the sequence set forth in SEQ ID NO:22 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:39 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:22 and the reverse primer is a contiguous sequence of nucleotides within a second region having the sequence set forth in SEQ ID NO:39.

In some embodiments, the forward primer is a contiguous sequence of nucleotides within a first region having the sequence set forth in SEQ ID NO:23 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:40 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:23 and the reverse primer is a contiguous sequence of nucleotides within a second region having the sequence set forth in SEQ ID NO:40.

In some embodiments, the forward primer is a contiguous sequence of nucleotides within a first region having the sequence set forth in SEQ ID NO:24 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:41 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:24 and the reverse primer is a contiguous sequence of nucleotides within a second region having the sequence set forth in SEQ ID NO:41.

In some embodiments, the forward primer is a contiguous sequence of nucleotides within a first region having the sequence set forth in SEQ ID NO:25 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:42 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:25 and the reverse primer is a contiguous sequence of nucleotides within a second region having the sequence set forth in SEQ ID NO:42.

In some embodiments, the forward primer is a contiguous sequence of nucleotides within a first region having the sequence set forth in SEQ ID NO:26 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:43 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:26 and the reverse primer is a contiguous sequence of nucleotides within a second region having the sequence set forth in SEQ ID NO:43.

In some embodiments, the forward primer is a contiguous sequence of nucleotides within a first region having the sequence set forth in SEQ ID NO:27 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:44 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:27 and the reverse primer is a contiguous sequence of nucleotides within a second region having the sequence set forth in SEQ ID NO:44.

In some embodiments, the forward primer is a contiguous sequence of nucleotides within a first region having the sequence set forth in SEQ ID NO:28 or a sequence having at least or about 85, 90, 91, 92, 93, 94, 95, 96, 97, 98, or 99% sequence identity thereto and the reverse primer is a contiguous sequence of nucleotides within a second region having the sequence set forth in SEQ ID NO:45 or a sequence having at least or about 85, 90, 91, 92, 93, 94, 95, 96, 97, 98, or 99% sequence identity thereto. In some embodiments, the forward primer is a contiguous sequence of nucleotides within a first region having the sequence set forth in SEQ ID NO:28 and the reverse primer is a contiguous sequence of nucleotides within a second region having the sequence set forth in SEQ ID NO:45.

In some embodiments, the forward and reverse oligonucleotide primer are each independently at least 15 nucleotides in length. In some embodiments, the forward and reverse oligonucleotide primer are each independently 15-30 nucleotides in length. In some embodiments, the forward and reverse oligonucleotide primer are each independently 18-25 nucleotides in length. In some embodiments, the forward and reverse oligonucleotide primer are each independently 18-22 nucleotides in length.

In some embodiments, the forward oligonucleotide primer comprises a sequence that has at least 80%, at least 85%, at least 90% or at least 95% sequence identity to any one of SEQ ID NOS: 3, 4 or 5. In some embodiments, the forward oligonucleotide primer comprises the sequence set forth in any of SEQ ID NOS: 3, 4 or 5. In some embodiments, the reverse oligonucleotide primer comprises a sequence that has at least 80%, at least 85%, at least 90% or at least 95% sequence identity to any one of SEQ ID NOS: 6 or 7. In some embodiments, the reverse oligonucleotide primer comprises the sequence set forth in any of SEQ ID NOS: 6 or 7. In some embodiments, the forward oligonucleotide primer is set forth in SEQ ID NO:3 and the reverse oligonucleotide primer is set forth in SEQ ID NO:6.

In some embodiments, the oligonucleotide probe is specific for a third region between the first region and the second region of the SIN viral vector. In some embodiments, the oligonucleotide probe is specific for a sequence present in the R and/or U5 region of the viral LTR. In some embodiments, the oligonucleotide probe comprises a sequence that has at least 80%, at least 85%, at least 90% or at least 95% sequence identity to any one of SEQ ID NOS: 8 or 9 or is a complement thereof. In some embodiments, the oligonucleotide probe comprises the sequences set forth in SEQ ID NOs 8 or 9 or is a complement thereof. In some embodiments, the oligonucleotide probe comprises the sequence as set forth in SEQ ID NO 8.

In some embodiments, probe further comprises a detectable moiety In some embodiments, the detectable moiety is fluorescent. In some embodiments, the detectable moiety is selected from FAM™ (Applera Corp.), HEX™ (Applera Corp.), FITC™ (Life Technologies, Inc), Texas Red™ (Molecular Probes, Inc.), TET™ (Life Technologies, Inc), VIC™ (Life Technologies, Inc), TAMRA™ (Applera Corp.), ROX™ (Applera Corp.), LIZ™ (Gene Scan, Inc), Cy3™ (Amersham Pharmacia Biotech), or Cy5™ (Amersham Pharmacia Biotech).

In some aspects, the kit can contain reagents and consumables required for polymerase chain reaction (PCR) or reverse-transcription PCR, such as for quantitative PCR (qPCR; also known as RT-PCR) or droplet digital PCR (ddPCR). In some embodiments, the kit can optionally contain other components including PCR regents such as polymerase, buffers, and free nucleotides.

In some embodiments, provided are kits that comprise at least one probe and/or one or more primers, such as a pair of primers, specific for at least a portion of the reverse-transcriptase dependent amplicon sequence. In some embodiments, the probes and/or primer can specifically bind to or recognize or detect all or a portion of the reverse-transcriptase dependent amplicon sequence. In some embodiments, the kit can contain reagents and consumables required for polymerase chain reaction (PCR), such as for quantitative PCR (qPCR) or droplet digital PCR (ddPCR).

In some embodiments, the kits optionally contain other components, for example: reagents such as polymerase enzymes, buffer, and/or nucleotides. The various components of the kit may be present in separate containers or certain compatible components may be precombined into a single container. In some embodiments, the kits further contain instructions for using the components of the kit to practice the provided methods.

### V. Exemplary Embodiments

Among provided embodiments are:

1. A method of amplifying a reverse-transcribed amplicon, the method comprising incubating DNA from a sample with (i) a forward oligonucleotide primer and a reverse oligonucleotide primer, wherein:
the primers are specific for a region of reverse-transcribed viral DNA of a self-inactivating (SIN) viral vector, and (ii) a DNA polymerase; and the incubating is under conditions sufficient for amplification of a reverse-transcriptase-dependent amplicon if the amplicon is present in the sample.

2. A method of detecting a reverse-transcribed amplicon, the method comprising:
(a) incubating DNA from a sample with (i) a forward oligonucleotide primer and a reverse oligonucleotide primer, wherein:
the primers are specific for a region of reverse-transcribed viral DNA of a self-inactivating (SIN) viral vector, and (ii) a DNA polymerase; and
the incubating is under conditions sufficient for amplification of a reverse-transcriptase-dependent amplicon if the amplicon is present in the sample; and
(b) detecting the generated reverse-transcriptase-dependent amplicon.

3. The method of embodiment 1 or 2, wherein the reverse-transcribed amplicon is reverse-transcriptase dependent.

4. The method of embodiment 1 or 2, wherein the incubation is performed by polymerase chain reaction (PCR).

5. The method of embodiment 4, wherein the PCR is quantitative PCR.

6. The method of any of embodiments 1-5, wherein the incubating is further with an oligonucleotide probe specific for the region of the reverse-transcribed viral DNA, wherein the oligonucleotide probe comprises a detectable moiety.

7. The method of embodiment 6, further comprising detecting a signal from the detectable moiety.

8. The method of embodiment 7, further comprising quantifying an amount of the generated reverse-transcriptase-dependent amplicon from the detected signal.

9. The method of any of embodiments 1-8, wherein the sample is known or suspected of containing viral nucleic acid from the SIN viral vector.

10. The method of embodiments 1-9, wherein the sample further comprises a known positive control, optionally wherein said positive control is a viral nucleic acid.

11. A method of quantifying a reverse-transcribed amplicon in a sample, the method comprising:
(a) performing quantitative PCR of DNA from a sample known or suspected of containing viral nucleic acid from a self-inactivating (SIN) viral vector by incubation of DNA from the sample with (i) a forward oligonucleotide primer and a reverse oligonucleotide primer, wherein the primers are specific for a region of reverse-transcribed viral DNA from the SIN viral vector, (ii) an oligonucleotide probe specific for the region of the reverse-transcribed viral DNA, wherein the oligonucleotide probe comprises a detectable moiety; and (iii) a DNA polymerase, wherein:
the quantitative PCR is performed under conditions sufficient for amplification of a reverse-transcriptase-dependent amplicon if the amplicon is present in the sample; and
(b) quantifying an amount of the generated reverse-transcriptase-dependent amplicon by detection of a detectable signal from the detectable moiety.

12. A method of determining viral vector copy number in a sample, the method comprising:
(a) performing quantitative PCR of DNA from a sample known or suspected of containing a viral nucleic acid from a self-inactivating viral vector by incubation of DNA from the sample with (i) a forward oligonucleotide primer and a reverse oligonucleotide primer, wherein the primers are specific for a regions of reverse-transcribed viral DNA from the SIN viral vector, (ii) an oligonucleotide probe specific for the region of the reverse-transcribed viral DNA, wherein the oligonucleotide probe comprises a detectable moiety, and (iii) a DNA polymerase, wherein:
the quantitative PCR is performed under conditions sufficient for amplification of a reverse-transcriptase-dependent amplicon if the amplicon is present in the sample;
(b) quantifying an amount of the generated reverse-transcriptase-dependent amplicon by detection of a detectable signal from the detectable moiety; and
(c) determining viral vector copy number in the sample.

13. The method of embodiment 11, wherein the reverse-transcribed amplicon is reverse-transcription dependent.

14. The method of any of embodiments 5-13, wherein the quantitative PCR is real-time PCR.

15. The method of any of embodiments 5-13, wherein the quantitative PCR is a digital PCR amplification.

16. The method of any of embodiments 5-13, wherein the quantitative PCR is digital droplet PCR (ddPCR).

17. The method of any of embodiments 1-10, wherein the SIN viral vector is a retroviral vector.

18. The method of any of embodiments 1-10, wherein the SIN viral vector is a gamma-retroviral vector.

19. The method of embodiment 17, wherein the retroviral vector is a lentiviral vector.

20. The method of embodiment 19, wherein the lentiviral vector is derived from HIV-1.

21. The method of any embodiments 1-20, wherein the forward oligonucleotide primer comprises a contiguous sequence of nucleotides of the plus strand of a first region that is within the deleted U3 (delU3) of the reverse-transcribed SIN viral vector, and the reverse oligonucleotide primer comprises a contiguous sequence of nucleotides of the minus strand of a second region downstream of the first region in the reverse-transcribed viral vector DNA to make a replicable reverse-transcriptase-dependent amplicon.

22. The method of embodiment 21, wherein the reverse-transcriptase-dependent amplicon is 50 base pairs to 500 base pairs in size.

23. The method of embodiment 21 or embodiment 22, wherein the reverse-transcriptase-dependent amplicon is 100 base pairs to 200 base pairs in size.

24. The method of any of embodiments 21-23, wherein the first region comprises a U3 attachment sequence.

25. The method of any of embodiments 21-24, wherein the first region comprises a sequence set forth in any one of SEQ ID NOS: 46-48.

26. The method of any of embodiments 21-25, wherein the forward primer comprises at least a contiguous portion of any one of SEQ ID NOS: 46-48.

27. The method of any of embodiments 21-26, wherein the first region comprises the sequence set forth in any one of SEQ ID NOS: 1 and 14-28 or a sequence having at least or about 85, 90, 91, 92, 93, 94, 95, 96, 97, 98, or 99% sequence identity to any one of SEQ ID NOS: 1 and 14-28.

28. The method of any of embodiments 21-27, wherein the first region is set forth in SEQ ID NO:1.

29. The method of any of embodiments 21-28, wherein the second region comprises at least a portion of a primer binding site (PBS), an IFN-stimulated response element (ISRE), and/or a Psi (Ψ) packaging sequence of the SIN viral vector.

30. The method of any of embodiments 21-29, wherein the second region comprises the sequence set forth in any one of SEQ ID NOS: 2 and 29-45 or a sequence having at least or about 85, 90, 91, 92, 93, 94, 95, 96, 97, 98, or 99% sequence identity to any one of SEQ ID NOS: 2 and 29-45.

31. The method of embodiments 21-30, wherein:
(i) the forward primer comprises a contiguous sequence of nucleotides within the sequence set forth in SEQ ID NO:1 or a sequence having at least or about 85, 90, 91, 92, 93, 94, 95, 96, 97, 98, or 99% sequence identity thereto, and the reverse primer comprises a contiguous sequence of nucleotides within the sequence set forth in SEQ ID NO:2 or a sequence having at least or about 85, 90, 91, 92, 93, 94, 95, 96, 97, 98, or 99% sequence identity thereto;
(ii) the forward primer comprises a contiguous sequence of nucleotides within g the sequence set forth in SEQ ID NO:14 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:29 or a sequence having at least or about 85, 90, 91, 92, 93, 94, 95, 96, 97, 98, or 99% sequence identity thereto;
(iii) the forward primer comprises a contiguous sequence of nucleotides within a the sequence set forth in SEQ ID NO:15 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:30 or a sequence having at least or about 85, 90, 91, 92, 93, 94, 95, 96, 97, 98, or 99% sequence identity thereto;
(iv) the forward primer comprises a contiguous sequence of nucleotides within the sequence set forth in SEQ ID NO:16 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:31 or a sequence having at least or about 85, 90, 91, 92, 93, 94, 95, 96, 97, 98, or 99% sequence identity thereto;
(v) the forward primer comprises a contiguous sequence of nucleotides within the sequence set forth in SEQ ID NO:17 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:32 or a sequence having at least or about 85, 90, 91, 92, 93, 94, 95, 96, 97, 98, or 99% sequence identity thereto;
(vi) the forward primer comprises a contiguous sequence of nucleotides within the sequence set forth in SEQ ID NO:18 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:33 or a sequence having at least or about 85, 90, 91, 92, 93, 94, 95, 96, 97, 98, or 99% sequence identity thereto;
(vii) the forward primer comprises a contiguous sequence of nucleotides within the sequence set forth in SEQ ID NO:19 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:35 or a sequence having at least or about 85, 90, 91, 92, 93, 94, 95, 96, 97, 98, or 99% sequence identity thereto;
(viii) the forward primer comprises a contiguous sequence of nucleotides within the sequence set forth in SEQ ID NO:20 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:36 or a sequence having at least or about 85, 90, 91, 92, 93, 94, 95, 96, 97, 98, or 99% sequence identity thereto;
(ix) the forward primer comprises a contiguous sequence of nucleotides within the sequence set forth in SEQ ID NO:21 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:38 or a sequence having at least or about 85, 90, 91, 92, 93, 94, 95, 96, 97, 98, or 99% sequence identity thereto;
(x) the forward primer comprises a contiguous sequence of nucleotides within the sequence set forth in SEQ ID NO:22 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:39 or a sequence having at least or about 85, 90, 91, 92, 93, 94, 95, 96, 97, 98, or 99% sequence identity thereto;
(xi) the forward primer comprises a contiguous sequence of nucleotides within the sequence set forth in SEQ ID NO:23 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:40 or a sequence having at least or about 85, 90, 91, 92, 93, 94, 95, 96, 97, 98, or 99% sequence identity thereto;
(xii) the forward primer comprises a contiguous sequence of nucleotides within the sequence set forth in SEQ ID NO:24 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:41 or a sequence having at least or about 85, 90, 91, 92, 93, 94, 95, 96, 97, 98, or 99% sequence identity thereto;
(xiii) the forward primer comprises a contiguous sequence of nucleotides within the sequence set forth in SEQ ID NO:25 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:42 or a sequence having at least or about 85, 90, 91, 92, 93, 94, 95, 96, 97, 98, or 99% sequence identity thereto;
(xiv) the forward primer comprises a contiguous sequence of nucleotides within the sequence set forth in SEQ ID NO:26 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:43 or a sequence having at least or about 85, 90, 91, 92, 93, 94, 95, 96, 97, 98, or 99% sequence identity thereto;
(xv) the forward primer comprises a contiguous sequence of nucleotides within the sequence set forth in SEQ ID NO:27 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:44 or a sequence having at least or about 85, 90, 91, 92, 93, 94, 95, 96, 97, 98, or 99% sequence identity thereto; or
(xvi) the forward primer comprises a contiguous sequence of nucleotides within the sequence set forth in SEQ ID NO:28 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:45 or a sequence having at least or about 85, 90, 91, 92, 93, 94, 95, 96, 97, 98, or 99% sequence identity thereto.

32. The method of any of embodiments 1-31, wherein the forward and reverse oligonucleotide primer are each independently at least 14 nucleotides in length.

33. The method of any of embodiments 1-32, wherein the forward and reverse oligonucleotide primer are each independently 14-30 nucleotides in length.

34. The method of any of embodiments 1-33, wherein the forward and reverse oligonucleotide primer are each independently 18-25 nucleotides in length.

35. The method of any of embodiments 1-34, wherein the forward and reverse oligonucleotide primer are each independently 18-22 nucleotides in length.

36. The method of any of embodiments 1-35, wherein the reverse-transcriptase-dependent amplicon comprises the sequence set forth in SEQ ID NO:10 or a sequence that has at least 80%, at least 85%, at least 90% or at least 95% sequence identity to SEQ ID NO:10.

37. The method of any of embodiments 1-36, wherein the reverse-transcriptase-dependent amplicon is set forth in SEQ ID NO:10.

38. The method of any of embodiments 1-37, wherein the forward oligonucleotide primer comprises a sequence that has at least 80%, at least 85%, at least 90% or at least 95% sequence identity to any one of SEQ ID NOS: 3, 4 or 5.

39. The method of any of embodiments 1-38, wherein the forward oligonucleotide primer comprises the sequence set forth in any of SEQ ID NOS: 3, 4 or 5.

40. The method of any of embodiments 1-39, wherein the reverse oligonucleotide primer comprises a sequence that has at least 80%, at least 85%, at least 90% or at least 95% sequence identity to any one of SEQ ID NOS: 6 or 7.

41. The method of any of embodiments 1-40, wherein the reverse oligonucleotide primer comprises the sequence set forth in any of SEQ ID NOS: 6 or 7.

42. The method of any of embodiments 1-41, wherein the forward oligonucleotide primer is set forth in SEQ ID NO:3 and the reverse oligonucleotide primer is set forth in SEQ ID NO:6.

43. The method of any of embodiments 4-42, wherein the oligonucleotide probe is specific for a third region between the first region and the second region of the reverse-transcribed SIN viral vector DNA.

44. The method of any of embodiments 6-43, wherein the oligonucleotide probe is specific for a sequence present in an R and/or U5 region of the viral LTR of the reverse-transcribed SIN viral vector DNA.

45. The method of any of embodiments 6-44, wherein the oligonucleotide probe comprises a sequence that has at least 80%, at least 85%, at least 90% or at least 95% sequence identity to any one of SEQ ID NOS: 8 or 9 or is a complement thereof.

46. The method of any of embodiments 6-45, wherein the oligonucleotide probe comprises the sequences as set forth in SEQ ID NOs 8 or 9 or is a complement thereof.

47. The method of any embodiments 6-46, wherein the oligonucleotide probe comprises the sequence as set forth in SEQ ID NO 8.

48. The method of any of embodiments 6-47, wherein the probe further comprises a detectable moiety.

49. The method of any of embodiments 6-48, wherein the detectable moiety is fluorescent.

50. The method of any of embodiments 6-49, wherein the detectable moiety is selected from FAM, HEX, FITC, Texas Red, TET, JOE, VIC, NED, TAMRA, ROX, ABY, PET, JUN, LIZ, Cy3, or Cy5.

51. The method of any of embodiments 1-50, wherein the one or more regions of reverse-transcribed viral vector DNA are not present in a non-self-inactivating viral vector.

52. The method of any of embodiments 1-51, wherein the one or more regions of reverse-transcribed viral vector DNA are not present in a wild-type virus or the wild-type from which the viral vector is derived.

53. The method of any of embodiments 1-52, wherein the one or more regions of reverse-transcribed viral vector DNA is only present after reverse-transcription.

54. The method of any of embodiments 1-53, wherein the reverse-transcriptase dependent amplicon is not produced from viral vector transduction residuals that are episomal in a cell transduced with the viral vector, optionally wherein said residuals are plasmids.

55. The method of any of embodiments 1-54, wherein the sample comprises one or more cells suspecting of comprising viral nucleic acid integrated in the genome.

56. The method of any of embodiments 1-55, wherein the sample comprises one more cells transduced with a viral vector.

57. The method of any of embodiments 4-56, wherein the PCR is a multiplex PCR and further comprises producing a reference amplicon of a reference gene in the same reaction.

58. The method of embodiment 57, wherein the reference gene is an endogenous gene known to be present in the genome of the cells in the sample.

59. The method of embodiment 58, wherein the reference gene is known to be present in one or two copies in a diploid genome.

60. The method of any of embodiments 57-59, wherein the reference gene is selected from hTert, β-actin, GAPDH, ribonuclease P/MRP subunit 30 (RPP30).

61. The method of any of embodiments 12-40, wherein the viral vector copy number is the copy number or the average copy number of the generated reverse-transcriptase-dependent amplicon per cell in the sample.

62. The method of any of embodiments 12-40, wherein the viral vector copy number is the copy number or the average copy number of the generated reverse-transcriptase-dependent amplicon per diploid genome.

63. The method of embodiment 62, wherein the vector copy number per diploid genome is calculated as the ratio between the number of copies of the generated reverse-transcriptase-dependent amplicon and the reference amplicon, optionally multiplied by two.

64. A composition comprising (i) a forward oligonucleotide primer that comprises a contiguous sequence of nucleotides of the plus strand of a first region present within the deleted U3 (delU3) of a reverse-transcribed self-inactivating (SIN) viral vector DNA, and (ii) a reverse oligonucleotide primer that comprises a contiguous sequence of nucleotides of the minus strand of a second region downstream of the first region in the reverse-transcribed viral vector DNA to make a replicable reverse-transcriptase-dependent amplicon.

65. The composition of embodiment 64, wherein the reverse-transcriptase-dependent amplicon is 100 base pairs to 500 base pairs in size.

66. The composition of embodiment 64 or embodiment 65, wherein the reverse-transcriptase-dependent amplicon is 200 base pairs to 400 base pairs in size.

67. The composition of any of embodiments 64-66, wherein the first region comprises the U3 attachment sequence.

68. The composition of any of embodiments 64-66, wherein the first region comprises the sequence set forth in any one of SEQ ID NOS: 46-48.

69. The composition of any of embodiments 64-68, wherein the forward primer comprises at least a contiguous portion of any one of SEQ ID NOS: 46-48.

70. The composition of any of embodiments 64-69, wherein the first region comprises the sequence set forth in any one of SEQ ID NOS: 1 and 14-28 or a sequence having at least or about 85, 90, 91, 92, 93, 94, 95, 96, 97, 98, or 99% sequence identity to any one of SEQ ID NOS: 1 and 14-28.

71. The composition of any of embodiments 64-70, wherein the first region is set forth in SEQ ID NO:1.

72. The composition of any of embodiments 64-71, wherein the second region comprises at least a portion of the primer binding site (PBS), the IFN-stimulated response element (ISRE), and/or the Psi (Ψ) packaging sequence of the SIN viral vector.

73. The composition of any of embodiments 64-72, wherein the second region comprises the sequence set forth in any one of SEQ ID NOS: 2 and 29-45 or a sequence having at least or about 85, 90, 91, 92, 93, 94, 95, 96, 97, 98, or 99% sequence identity to any one of SEQ ID NOS: 2 and 29-45.

74. The composition of any of embodiments 64-73, wherein:
(i) the forward primer comprises a contiguous sequence of nucleotides within the sequence set forth in SEQ ID NO:1 or a sequence having at least or about 85, 90, 91, 92, 93, 94, 95, 96, 97, 98, or 99% sequence identity thereto, and the reverse primer comprises a contiguous sequence of nucleotides within the sequence set forth in SEQ ID NO:2 or a sequence having at least or about 85, 90, 91, 92, 93, 94, 95, 96, 97, 98, or 99% sequence identity thereto;
(ii) the forward primer comprises a contiguous sequence of nucleotides within the sequence set forth in SEQ ID NO:14 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:29 or a sequence having at least or about 85, 90, 91, 92, 93, 94, 95, 96, 97, 98, or 99% sequence identity thereto;
(iii) the forward primer comprises a contiguous sequence of nucleotides within the sequence set forth in SEQ ID NO:15 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:30 or a sequence having at least or about 85, 90, 91, 92, 93, 94, 95, 96, 97, 98, or 99% sequence identity thereto;
(iv) the forward primer comprises a contiguous sequence of nucleotides within the sequence set forth in SEQ ID NO:16 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:31 or a sequence having at least or about 85, 90, 91, 92, 93, 94, 95, 96, 97, 98, or 99% sequence identity thereto;
(v) the forward primer comprises a contiguous sequence of nucleotides within the sequence set forth in SEQ ID NO:17 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:32 or a sequence having at least or about 85, 90, 91, 92, 93, 94, 95, 96, 97, 98, or 99% sequence identity thereto;
(vi) the forward primer comprises a contiguous sequence of nucleotides within the sequence set forth in SEQ ID NO:18 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:33 or a sequence having at least or about 85, 90, 91, 92, 93, 94, 95, 96, 97, 98, or 99% sequence identity thereto;
(vii) the forward primer comprises a contiguous sequence of nucleotides within the sequence set forth in SEQ ID NO:19 or a sequence having at least or about 85, 90, 91, 92, 93, 94, 95, 96, 97, 98, or 99% sequence identity thereto and the reverse primer comprises a contiguous sequence of nucleotides within a the sequence set forth in SEQ ID NO:35 or a sequence having at least or about 85, 90, 91, 92, 93, 94, 95, 96, 97, 98, or 99% sequence identity thereto;
(viii) the forward primer comprises a contiguous sequence of nucleotides within the sequence set forth in SEQ ID NO:20 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:36 or a sequence having at least or about 85, 90, 91, 92, 93, 94, 95, 96, 97, 98, or 99% sequence identity thereto;
(ix) the forward primer comprises a contiguous sequence of nucleotides within the sequence set forth in SEQ ID NO:21 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:38 or a sequence having at least or about 85, 90, 91, 92, 93, 94, 95, 96, 97, 98, or 99% sequence identity thereto;
(x) the forward primer comprises a contiguous sequence of nucleotides within the sequence set forth in SEQ ID NO:22 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:39 or a sequence having at least or about 85, 90, 91, 92, 93, 94, 95, 96, 97, 98, or 99% sequence identity thereto;
(xi) the forward primer comprises a contiguous sequence of nucleotides within the sequence set forth in SEQ ID NO:23 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:40 or a sequence having at least or about 85, 90, 91, 92, 93, 94, 95, 96, 97, 98, or 99% sequence identity thereto;
(xii) the forward primer comprises a contiguous sequence of nucleotides within the sequence set forth in SEQ ID NO:24 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:41 or a sequence having at least or about 85, 90, 91, 92, 93, 94, 95, 96, 97, 98, or 99% sequence identity thereto;
(xiii) the forward primer comprises a contiguous sequence of nucleotides within the sequence set forth in SEQ ID NO:25 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:42 or a sequence having at least or about 85, 90, 91, 92, 93, 94, 95, 96, 97, 98, or 99% sequence identity thereto;
(xiv) the forward primer comprises a contiguous sequence of nucleotides within the sequence set forth in SEQ ID NO:26 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:43 or a sequence having at least or about 85, 90, 91, 92, 93, 94, 95, 96, 97, 98, or 99% sequence identity thereto;
(xv) the forward primer comprises a contiguous sequence of nucleotides within the sequence set forth in SEQ ID NO:27 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:44 or a sequence having at least or about 85, 90, 91, 92, 93, 94, 95, 96, 97, 98, or 99% sequence identity thereto; or
(xvi) the forward primer comprises a contiguous sequence of nucleotides within the sequence set forth in SEQ ID NO:28 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:45 or a sequence having at least or about 85, 90, 91, 92, 93, 94, 95, 96, 97, 98, or 99% sequence identity thereto.

75. The composition of any of embodiments 64-74, wherein the forward and reverse oligonucleotide primer are each independently at least 15 nucleotides in length.

76. The composition of any of embodiments 64-75, wherein the forward and reverse oligonucleotide primer are each independently 15-30 nucleotides in length.

77. The composition of any of embodiments 64-76, wherein the forward and reverse oligonucleotide primer are each independently 18-25 nucleotides in length.

78. The composition of any of embodiments 64-77, wherein the forward and reverse oligonucleotide primer are each independently 18-22 nucleotides in length.

79. The composition of any of embodiments 64-78, wherein the reverse-transcriptase-dependent amplicon comprises the sequence set forth in SEQ ID NO:10 or a sequence that has at least 80%, at least 85%, at least 90% or at least 95% sequence identity to SEQ ID NO:10.

80. The composition of any of embodiments 64-79, wherein the reverse-transcriptase-dependent amplicon is set forth in SEQ ID NO:10.

81. The composition of any of embodiments 64-80, wherein the forward oligonucleotide primer comprises a sequence that has at least 80%, at least 85%, at least 90% or at least 95% sequence identity to any one of SEQ ID NOS: 3, 4 or 5.

82. The composition of any of embodiments 64-81, wherein the forward oligonucleotide primer comprises the sequence set forth in any of SEQ ID NOS: 3, 4 or 5.

83. The composition of any of embodiments 64-82, wherein the reverse oligonucleotide primer comprises a sequence that has at least 80%, at least 85%, at least 90% or at least 95% sequence identity to any one of SEQ ID NOS: 6 or 7.

84. The composition of any of embodiments 64-83, wherein the reverse oligonucleotide primer comprises the sequence set forth in any of SEQ ID NOS: 6 or 7.

85. The composition of any of embodiments 64-84, wherein the forward oligonucleotide primer is set forth in SEQ ID NO:3 and the reverse oligonucleotide primer is set forth in SEQ ID NO:6.

86. The composition of any of embodiments 64-85, wherein the composition further comprises a polymerase enzyme.

87. The composition of embodiment 86, wherein the polymerase enzyme is a DNA polymerase, optionally a Taq DNA polymerase, Hot Start DNA polymerase and/or a high fidelity DNA polymerase.

88. The composition of any of embodiments 64-87, wherein the composition further comprises an oligonucleotide probe.

89. The composition of embodiment 88, wherein the oligonucleotide probe is specific for a sequence present in an R and/or U5 region of the viral LTR.

90. The composition of embodiment 88 or embodiment 89, wherein the oligonucleotide probe comprises a sequence that has at least 80%, at least 85%, at least 90% or at least 95% sequence identity to any one of SEQ ID NOS: 8 or 9 or is a complement thereof.

91. The composition of any of embodiments 88-90, wherein the probe comprises the nucleic acid sequence set forth in any of SEQ ID NOs 8 or 9.

92. The composition of any of embodiments 88-91, wherein the probe further comprises a detectable moiety, optionally wherein said moiety is fluorescent.

93. The composition of embodiment 92, wherein the detectable moiety is selected from FAM, HEX, FITC, Texas Red, TET, VIC, TAMRA, ROX, LIZ, Cy3, or Cy5.

94. A probe, comprising a nucleic acid sequence set forth in any of SEQ ID NOs. 8 or 9.

95. The probe of embodiment 94, wherein the probe further comprises a detectable moiety.

96. The probe of embodiment 95, wherein the detectable moiety is fluorescent.

97. The probe of embodiment 95 or embodiment 96, wherein the detectable moiety is selected from FAM, HEX, FITC, Texas Red, TET, VIC, TAMRA, ROX, LIZ, Cy3, or Cy5.

98. A kit, comprising the composition of any one of embodiments 64-93 and/or any one of the probes of embodiments 94-97.

99. A kit, comprising (i) a forward oligonucleotide primer comprising a nucleic acid sequence set forth in any of SEQ ID NOs 3-5, (ii) a reverse oligonucleotide primer comprising a nucleic acid sequence set forth in any of SEQ ID NOs 6-7, and (iii) a probe comprising a nucleic acid sequence set forth in any of SEQ ID NOs 8 or 9.

100. The kit of embodiment 99, wherein (i) the forward oligonucleotide primer comprises a nucleic acid sequence set forth in SEQ ID NO. 3, (ii) the reverse oligonucleotide primer comprises a nucleic acid sequence set forth in SEQ ID NO. 7, and (iii) the probe comprises a nucleic acid sequence set forth in SEQ ID NO. 8.

101. A reaction mixture, comprising the composition of any of embodiments 64-93, wherein the mixture further comprises a sample.

102. The reaction mixture of embodiment 101, wherein the sample comprises DNA from one more cells transduced with a self-inactivating (SIN) viral vector.

103. The reaction mixture of embodiment 101 or embodiment 102, wherein the sample comprises DNA from one or more cells suspecting of comprising SIN viral nucleic acid integrated in the genome.

104. The reaction mixture of any of embodiments 101-103, wherein the reaction mixture further comprises all four deoxyribonucleoside triphosphates, and/or an appropriate buffer, optionally wherein said buffer comprises Mn+2.

### VI. Examples

The following examples are included for illustrative purposes only and are not intended to limit the scope of the invention.

### Example 1 Optimization of Reverse-Transcription Dependent Amplicon for Detection of Vector Copy Number

This Example describes design of a reverse-transcription (RT) dependent amplicon and corresponding methods of amplification and detection of reverse-transcribed (R-T) self-inactivating (SIN) viral vector nucleic acids. The amplicon and related methods can be used for determination of vector copy number (VCN) of SIN lentiviral vectors (LV) even in the presence of potential LV production residuals such as plasmids.

### A. Design of R-T Specific Amplicons

A series of primers were first constructed so as to be specific to only reverse-transcribed self-inactivating (SIN) viral vector nucleic acids. Unique to the replication strategy of SIN viral vectors, such as HIV-based LVs, is an initial priming by a tRNA such that the 5′ U5 region is used to prime the 3′ end of the genome following the first strand transfer (Coffin, J. M., Hughes, S. H. and Varmus, H. Cold Spring Harbor Laboratory Press, 1997.). This strand transfer of the U3 to the 5′ end results in duplicate repeats. Here, forward primers were designed within a region of the 3′ U3 and reverse primers within the 5′ SIN sequence to be specific for a unique “DelU3” amplicon which can only exist after this R-T dependent strand transfer and would not be expected to be present on any bystander molecules or production residuals. Probes also were designed that were complementary a region of the same strand or the reverse complement strand and that did not overlap with a primer-binding site on the same strand, however, in some cases the probe also may be designed to target in the R and U5 region of the LTR. Thus the detection of only reverse-transcribed nucleic acid lentiviral vector products relies on the specific orientation of primers and probes to only form an amplicon after reverse transcription has been completed.

Specifically, exemplary primers and probes were designed for detection of a DelU3 amplicon (SEQ ID NO:10) in an exemplary HIV1-based lentivirus expression vector.

| | |
|---|---|
| DelU3 | CTGGAAGGGCTAATTCACTCCCAACGAAGACAAGATC |
| Amplicon | TGCTTTTTGCTTGTACTGGTCTCTCTGGTTAGACCAGA |
| | TCTGAGCCTGGGAGCTCTCTGGCTAACTAGGGAACCC |
| | ACTGCTTAAGCCTCAATAAAGCTTGCCTTGAGTGCTTC |
| | AAGTAGTGTGTGCCCGTCTGTTGTGTGACTCTGGTAAC |
| | TAGAGATCCCTCAGACCCTTTTAGTCAGTGTGGAAAAT |
| | CTCTAGCAGTGGCGCCCGAACAGGGACCTGAAAGCGA |
| | AAGGGAAACC (SED ID NO: 10) |

Candidate forward primers for the exemplary amplicon include any 18-25 base pair primer complementary to a region containing a 3′ U3 sequence set forth in SEQ ID NO:1 (TGGAAGGGCTAATTCACTCCCAACGAAGACAAGATCTGCTTTTTGCTTGTACTGG) and candidate reverse primers include any 18-25 base pair primer complementary to a region containing the 5′ sequence set forth in SEQ ID NO:2 (CTGAAAGCGAAAGGGAAACCAGAGCTCTCTCGAC). Exemplary forward and reverse primers and probes are as set forth in Table E1 below. The probes contained a fluorescein (FAM) dye label for detection.

TABLE E1
Exemplary Primers and Probes for a R-T
Specific DelU3 Amplicon
Exemplary Forward Primers

| | |
|---|---|
| FWD 1 | GGAAGGGCTAATTCACTCCC (SED ID NO: 3) |
| FWD 2 | CCCAACGAAGACAAGATCTGC (SED ID NO: 4) |
| FWD 3 | GAGCTCGGTACCTTTAAGACC (SED ID NO: 5) |

Exemplary Reverse Primer

| | |
|---|---|
| REV 1 | CCTGAAAGCGAAAGGGAAACC (SED ID NO: 6) |
| REV 2 | GGTTTCCCTTTCGCTTTCAGG (SED ID NO: 7) |

Exemplary Probes

| | |
|---|---|
| Probe 1 | TGCCCGTCTGTTGTGTGACTCTG (SED ID NO: 8) |
| (FAM) | |
| Probe 2 | TGCTTGTACTGGGTCTCTCTGG (SED ID NO: 9) |
| (FAM) | |

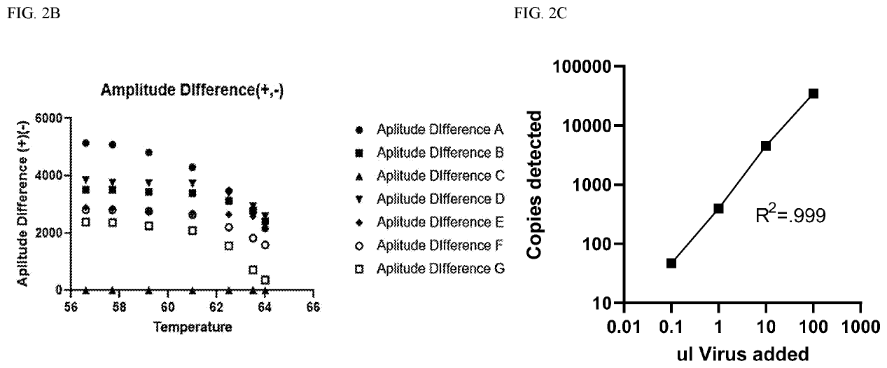

Exemplary forward and reverse primer and probes were then randomly paired via a matrix and used to assess recovery and separation of seven alternative amplicons of the DelU3 amplicon using digital droplet PCR (ddPCR) (Table E2). For normalization, a reference primer probe set targeting the exemplary reference gene hTert also was amplified in the reaction, in which the target amplicon was detected by the FAM probe and the reference amplicon was detected by a HEX probe. DdPCR converts a liquid sample into many tens of thousands individual droplets in oil emersion, allowing each droplet to represent a separate amplification reaction. Following PCR amplification of the nucleic acid in the droplets, the plate containing the droplets was placed into a droplet reader for analysis using a two-color detection system (set to detect FAM and HEX). Exemplary results are shown in FIG. 2A, in which droplets positive for the FAM signal are shown in the top panel and in which droplets that are single or double positive for the target amplicon and the reference amplicon are shown in the bottom panel.

TABLE E2
Amplicon A-G Primer/Probe Conditions

| Primer Set | Primer and Probes |
|---|---|
| Amplicon A | FWD 1 + REV 1 + Probe 2 |
| Amplicon B | FWD 1 + REV 1 + Probe 1 |
| Amplicon C | FWD 2 + REV 1 + Probe 2 |
| Amplicon D | FWD 2 + REV 1 + Probe 1 |
| Amplicon E | FWD 3 + REV 1 + Probe 2 |
| Amplicon F | FWD 3 + REV 1 + Probe 1 |
| Amplicon G | FWD 1 + REV 1 + Probe 1 + Probe 2 |

Random primer pairs A-G were tested for amplitude differences yielded across a thermogradient. As shown in FIG. 2B, all of the randomly paired primers, except Primer Pair C, yielded amplitude differences that were quantifiable (i.e., difference in amplitude between a droplet negative for the target, and one positive for the target). It was expected that Primer Pair C would not yield detectable amplification, since here the probe was upstream of the forward primer. Primer set B was observed to amplify an Amplicon B with acceptable recovery and separation of positive and negative droplets across the temperature gradient.

Primer set B was further tested for amplification of the delU3 amplicon by ddPCR at a temperature of 60.3° C. across an LV dosage range standardly used for transduction. As shown in FIG. 2C, detection of the amplicon following amplification with primer set B was linear across a usual vector dosage range.

### B. Detection of DelU3 Amplicon Compared to WPRE Amplicon

To assess if detection of DelU3 amplicon was specific to integrated vector, the method of amplification described above was compared to methods that rely on amplification of a Woodchuck Hepatitis Virus (WHV) Posttranscriptional Regulatory Element (WPRE) amplicon. WPRE is a DNA sequence commonly used in vector construction to increase expression of the transgene, and is therefore present on production residuals like plasmids. This stands in contrast to the strategy as disclosed herein whereby the DelU3 amplicon is specific to sequences which have undergone reverse transcription (i.e., not free nucleic acid or nucleic acid as is present on a plasmid).

VSV-G pseudotyped lentiviral vector (VG) was used to transduce cells and ddPCR of nucleic acid from transduced cells was carried out by amplification of the WPRE amplicon or the DelU3 amplicon (also called LTR). For detection of the WPRE amplicon, the forward and reverse primers were as set forth in SEQ ID NO. 11 and 12, respectively, and the corresponding probe was as is set forth in SEQ ID NO. 13. As a comparison, the DelU3 amplicon was amplified and detected using primer set B and probes as described above. Two conditions for amplification of each amplicon were tested, a condition including 30 μM of the drug Nevirapine and an untreated control. Nevirapine (Nev) is a non-nucleoside reverse transcriptase inhibitor (NNRTI) and works to prevent the action of the reverse transcriptase enzyme. Vector copy number was determined by assessing the number of copies of the detected amplicon per diploid genome.

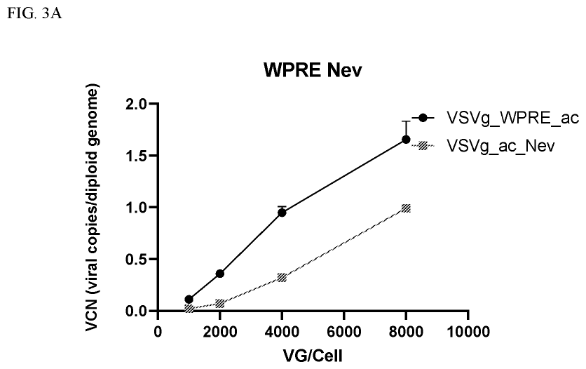

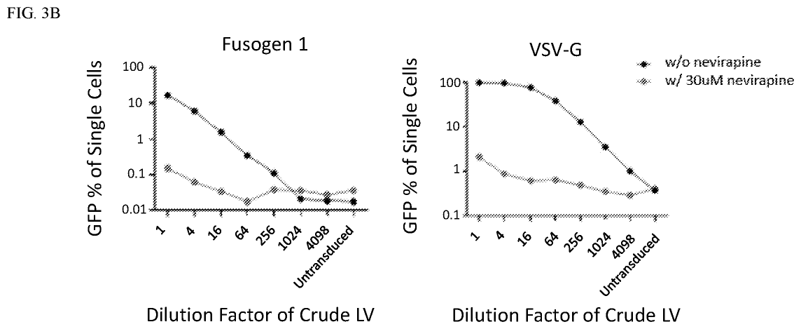

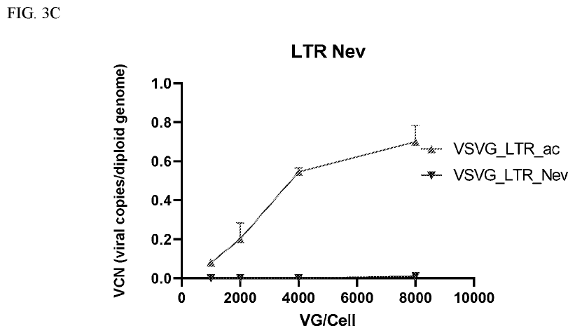

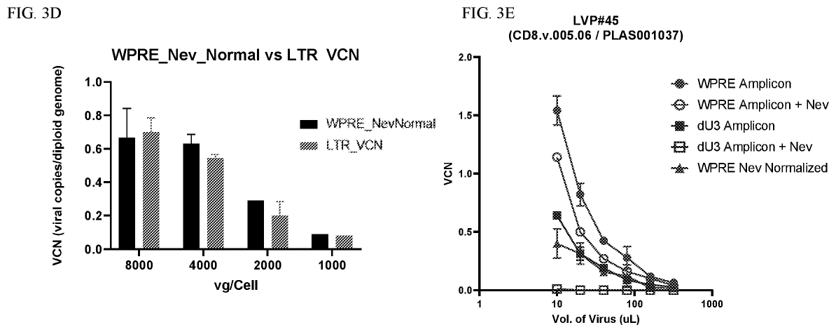

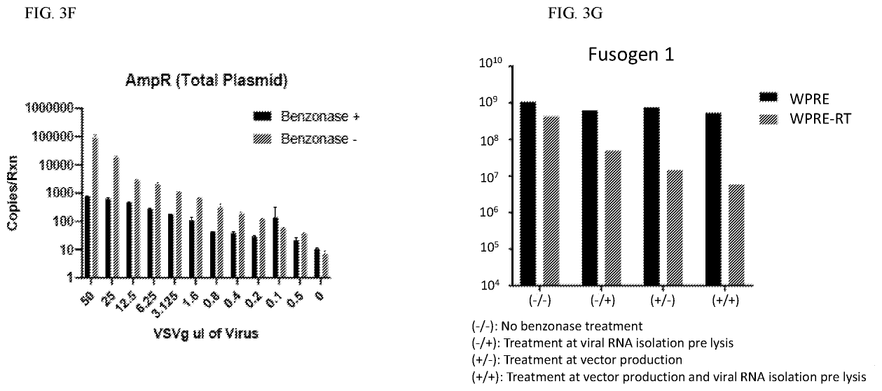

As shown in FIG. 3A determination of VCN based on assay of the WPRE amplicon was not substantially impacted in the presence of Nevirapine treatment, indicating this amplicon is not specific for reverse transcribed nucleic acids and is reporting presence of production residuals which also comprise the WPRE sequence. These data are further supported by analysis of GFP expression as a result of successful expression of the GFP reporter transgene present on the exemplary integrated vector, as shown for a VSV-G pseudotyped lentiviral vector as well as for a lentiviral vector pseudotyped with a CD8 re-targeted Paramyxovirus fusogen (designated “fusogen 1”) (FIG. 3B). In contrast, as shown in FIG. 3C, determination of VCN based on assay of the DelU3 amplicon with Nevirapine treatment completely ablated detection of VCN. These results demonstrate that the delU3 amplicon is Nevirapine independent and reverse-transcription dependent.

Since the WPRE amplicon may detect residual plasmid, to confirm true vector copy integration into the genome it is necessary to perform Nevirapine normalization by subtracting copies detected with a Nevirapine control treatment from copied detected in samples that has not been treated with Nevirapine. This normalization is shown in FIG. 3D for the conditions labeled WPRE_NevNormal. Strikingly, FIG. 3C further shows that the delU3 amplicon resulted in a similar VCN in samples that were not treated with Nevirapine. Similar results are shown in FIG. 3E in which VCN was calculated to be substantially the same using an assay detecting the WPRE amplicon followed by Nev normalization (WPRE Nev normalization) or using an assay detecting the delU3 amplicon without any need for normalization. These results further confirm that the delU3 amplicon is detecting only integrated vector nucleic acid, and that assaying for integrated VCN does not require normalization.

In some aspects, another method that can be used to reduce nucleic acid impurity (i.e., residual plasmids and nucleic acids) is via treatment with a benzonase enzyme. Benzonase is a bacterial enzyme which cleaves all forms of DNA and RNA (e.g., single-stranded, double-stranded, linear, and circular) without sequence specificity. Benzonase digests via hydrolysis of nucleic acid sequences into smaller oligonucleotides, in some aspects these smaller nucleotides are of <10 base pairs in length. As shown in FIG. 3F, there is still significant plasmid residual observed following benzonase treatment, including during isolation of the viral RNA or production of the vector (FIG. 3G).

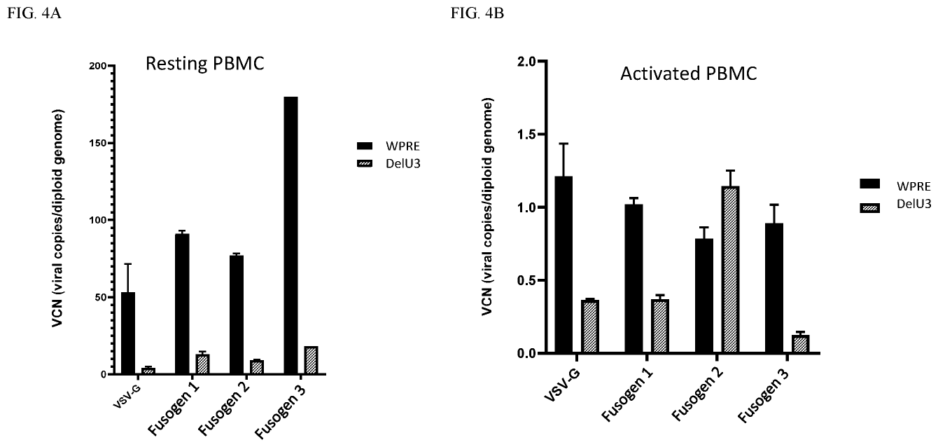

To assess the utility of the delU3 amplicon assay in primary T cells, resting PBMC or activated PBMCs were transduced with different pseudotyped lentiviral vectors and VCN was determined using an assay to detect the WPRE amplicon or DelU3 amplicon as described above. In both assays, Nevirapine treatment was not used. As shown in FIG. 4A (resting PBMCs) and FIG. 4B (activated PBMCs) a higher VCN of transduced cells was generally observed by detection of the WPRE amplicon compared to the DelU3 amplicon. This result further confirms that non-RT dependent amplification from residual plasmids or free nucleotides results in an overestimation of VCN of 50-150x as compared to detection of the DelU3 amplicon.

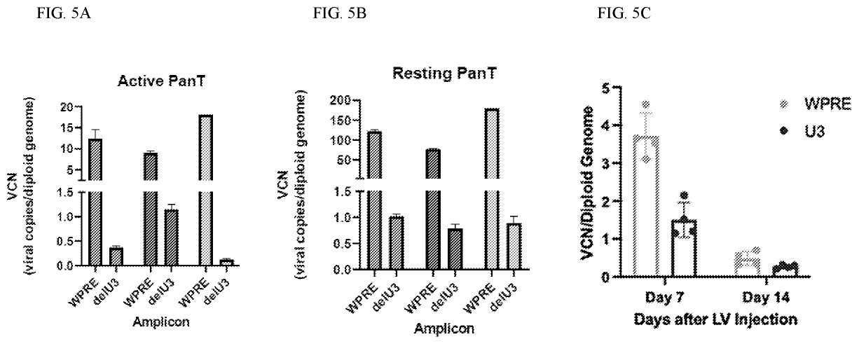

In a similar experiment, Pan T cells or activated Pan T cells expressing CD3+/CD4+ and/or CD3+/CD8+ were transduced with different pseudotyped lentiviral vectors and VCN was determined using an assay to detect the WPRE amplicon or DelU3 amplicon as described above. Vectors were pseudotyped with one of three exemplary CD8-retargeted fusogens. As shown in FIG. 5A (Active Pan T) and FIG. 5B (resting Pan T), an overestimated VCN of transduced cells was generally observed on Day 6 post transduction by detection of the WPRE amplicon compared to the DelU3 amplicon. These data are further supported by FIG. 5C, wherein the VCN of isolated murine hepatocytes was also observed to be overestimated on Day 7 post IV administration of the lentiviral vector. Taken together these data support that the DelU3 amplicon is specific for reverse transcribed sequences where typical amplification of WPRE is not.

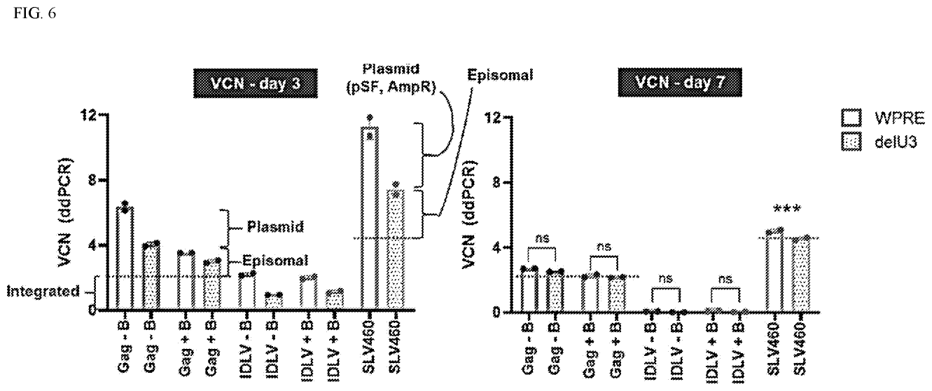

Next, the longevity of detection of DelU3 was assessed using SupT1 cells transduced with one of two VSV-G pseudotyped viruses, one which comprised a wild type integrase and one which was integration deficient (IDLV). Stable signal of the DelU3 amplicon was detected by gel electrophoresis over at least a 13-day period in cells transduced with integration-capable virus, and was lost by day 6 post transduction with integration-deficient virus. As expected, episomal (i.e., not genomically integrated) signal was substantially lost over the two week study period. A graph of VCN as determined by ddPCR is also shown in FIG. 6. A VSVg pseudotyped lentiviral vector with the same transfer genome, known as SLV, was used as a viral production assay control. VCN determined on Day 3 (left panel) was observed to include nucleic acid that was integrated, as well as those present as residuals (i.e., plasmid and episomal). VCN determination by Day 7 (right panel) showed no significant difference between WPRE or delU3 utilization, as nonintegrated nucleic acids are lost over time.

In sum, these data support the use of a reverse-transcription dependent amplicon DelU3 and corresponding methods of detection for VCN.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

VII. SEQUENCES

| SEQ | | |
|---|---|---|
| ID | | |
| NO | SEQUENCE | Annotation |
| 1 | TGGAAGGGCTAATTCACTCCCAACGAAGACAAGATCTGCTT | Region for Forward |
| | TTTGCTTGTACTGG | Primers |
| 2 | CTGAAAGCGAAAGGGAAACCAGAGCTCTCTCGAC | Region for Reverse |
| | | Primers |
| 3 | GGAAGGGCTAATTCACTCCC | FWD 1 |
| 4 | CCCAACGAAGACAAGATCTGC | FWD 2 |
| 5 | GAGCTCGGTACCTTTAAGACC | FWD 3 |
| 6 | CCTGAAAGCGAAAGGGAAACC | REV 1 |
| 7 | GGTTTCCCTTTCGCTTTCAGG | REV 2 |
| 8 | TGCCCGTCTGTTGTGTGACTCTG | Probe 1 |
| 9 | TGCTTGTACTGGGTCTCTCTGG | Probe 2 |
| 10 | CTGGAAGGGCTAATTCACTCCCAACGAAGACAAGATCTGC | DelU3 Amplicon |
| | TTTTTGCTTGTACTGGTCTCTCTGGTTAGACCAGATCTGAG | |
| | CCTGGGAGCTCTCTGGCTAACTAGGGAACCCACTGCTTAA | |
| | GCCTCAATAAAGCTTGCCTTGAGTGCTTCAAGTAGTGTGT | |
| | GCCCGTCTGTTGTGTGACTCTGGTAACTAGAGATCCCTCA | |
| | GACCCTTTTAGTCAGTGTGGAAAATCTCTAGCAGTGGCGC | |
| | CCGAACAGGGACCTGAAAGCGAAAGGGAAACC | |
| 11 | ACTTTCGCTTTCCCCCTCC | WPRE FWD |
| 12 | CGGAATTGTCAGTGCCCAAC | WPRE REV |
| 13 | CCACGGCGGAACTCATCG | WPRE Probe |
| 14 | TGGAAGGGCTAATTCACTCCCAAAGAAGACAAGAT | pHIV-7 (Forward) |
| 15 | TGGAAGGGCTAATTCACTCCCAACGAAGACAAGATATC | pTNS9 (Forward) |
| | CTTGATCTGTGGATCTA | |
| 16 | TGGAAGGGCTAATTCACTCCCAACGAAGACAAGAT | pWPXL (Forward) |
| 17 | TGGAAGGGCTAATTCACTCCCAACGAAGACAAGAT | pRRL (Forward) |
| 18 | TGGAAGGGCTAATTCACTCCCAACGAAGACAAGAT | pCCL (Forward) |
| 19 | TGGAAGGGCTAATTCACTCCCAACGAAGACAAGATATCC | pNL 'U3 (Forward) |
| | TTGATCTGTGGATCTA | |
| 20 | TGGAAGGGCTAATTCACTCCCAAAGAAGACAA | pCSCIGW (Forward) |
| 21 | TGGAAGGGCTAATTCACTCCCAACGAAGATAAGAT | pRSIEG (Forward) |
| 22 | TGGAAGGGCTAATTCACTCCCAACGAAGACAAGAT | pHage (Forward) |
| 23 | TGGAAGGGCTAATTCACTCCCAACGAAGACAAGAT | pSF Lenti (Forward) |
| 24 | TGGAAGGGCTAATTCACTCCCAACGAAGACAAGAT | pLenti7.3 (Forward) |
| 25 | TGGAAGGGCTAGCTCACTCCCAACGAAGACAAGAT | P156RRLsinppt |
| | | (Forward) |
| 26 | TGGAAGGGCTAATTCACTCCCA | pLL3.7m (Forward) |
| 27 | TGGAAGGGCTAATTCACTCCCAACGAAGACAAGATATC | pLV-GFPSpark |
| | CTTGATCTGTGGATCTA | (Forward) |
| 28 | TGGAAGGGCTAATTCACTCCCAACGAAGACAAGATATC | pLVX (Forward) |
| | CTTGATCTGTGGATCTA | |
| 29 | CTGAAAGCGAAAGGGAAAGCAGAGGAGCTCTCTCGAC | pHIV-7 (Group VI) |
| | | (Reverse) |
| 30 | CTGAAAGCGAAAGGGAAAGCAGAGGAGCTCTCTCGAC | pTNS9 (Group III) |
| | | (Reverse) |
| 31 | CTGAAAGCGAAAGGGAAAGCAGAGGAGCTCTCTCGAC | pWPXL (Group II) |
| | | (Reverse) |
| 32 | CCGAAAGCGAAAGGGAAAGCAGAGCTCTCTCGAC | pRRL(Group I) |
| | | (Reverse) |
| 33 | CCGAAAGCGAAAGGGAAAGCAGAGCTCTCTCGAC | pCCL (Group I) |
| | | (Reverse) |
| 34 | CTGAAAGCGAAAGTAAAAGCAGAGGAGATCTCTCGAC | pTYF (Group VII) |
| | | (Reverse) |
| 35 | CTGAAAGCGAAAGTAAAAGCAGAGGAGATCTCTCGAC | pNL ′U3 (Group V) |
| | | (Reverse) |
| 36 | CTGAAAGCGAAAGGGAAAGCAGAGGAGCTCTCTCGAC | pCSCIGW (Group II) |
| | | (Reverse) |
| 37 | CTGAAAGCGAAAGGGAAAGCAGAGGAGCTCTCTCGAC | pLenti-III (Group A) |
| | | (Reverse) |
| 38 | CTGAAAGCGAAAGGGAAAGCAGAGGAGCTCTCTCGAC | pRSIEG (Group C) |
| | | (Reverse) |
| 39 | CTGAAAGCGAAAGGGAAAGCAGAGGAGCTCTCTCGAC | pHage (Group A) |
| | | (Reverse) |
| 40 | CTGAAAGCGAAAGGGAAAGCAGAGGAGCTCTCTCGAC | pSF Lenti (Group C) |
| | | (Reverse) |
| 41 | CTGAAAGCGAAAGGGAAAGCAGAGGAGCTCTCTCGAC | pLenti7.3 (Group C) |
| | | (Reverse) |
| 42 | CTGAAAGCGAAAGGGAAAGCAGAGGAGCTCTCTCGAC | P156RRLsinppt (Group |
| | | C) (Reverse) |
| 43 | CTGAAAGCGAAAGGGAAAGCAGAGGAGCTCTCTCGAC | pLL3.7m (Group A) |
| | | (Reverse) |
| 44 | CTGAAAGCGAAAGGGAAAGCAGAGGAGCTCTCTCGAC | pLV-GFPSpark (Group |
| | | A) (Reverse) |
| 45 | CTGAAAGCGAAAGGGAAAGCAGAGGAGCTCTCTCGAC | pLVX (Group A) |
| | | (Reverse) |
| 46 | TGGAAGGGCTAATTCACTCCCAAAGAA | U3 att seq 1 |
| 47 | TGGAAGGGCTAATTCACTCCCAACGAA | U3 att seq 2 |
| 48 | TGGAAGGGCTAGCTCACTCCCAACGAA | U3 att seq 3 |

What is claimed:

1. A method of amplifying a reverse-transcribed amplicon, the method comprising incubating DNA from a sample with (i) a forward oligonucleotide primer and a reverse oligonucleotide primer, wherein the primers are specific for a region of reverse-transcribed viral DNA of a self-inactivating (SIN) viral vector, and (ii) a DNA polymerase; and
the incubating is under conditions sufficient for amplification of a reverse-transcriptase-dependent amplicon if the amplicon is present in the sample.

2. A method of detecting a reverse-transcribed amplicon, the method comprising:
(a) incubating DNA from a sample with (i) a forward oligonucleotide primer and a reverse oligonucleotide primer, wherein the primers are specific for a region of reverse-transcribed viral DNA of a self-inactivating (SIN) viral vector, and (ii) a DNA polymerase; and
the incubating is under conditions sufficient for amplification of a reverse-transcriptase-dependent amplicon if the amplicon is present in the sample; and
(b) detecting the generated reverse-transcriptase-dependent amplicon.

3. The method of claim 1 or 2, wherein the reverse-transcribed amplicon is reverse-transcriptase dependent.

4. The method of claim 1 or 2, wherein the incubation is performed by polymerase chain reaction (PCR).

5. The method of claim 4, wherein the PCR is quantitative PCR.

6. The method of any of claims 1-5, wherein the incubating is further with an oligonucleotide probe specific for the region of the reverse-transcribed viral DNA, wherein the oligonucleotide probe comprises a detectable moiety.

7. The method of claim 6, further comprising detecting a signal from the detectable moiety.

8. The method of claim 7, further comprising quantifying an amount of the generated reverse-transcriptase-dependent amplicon from the detected signal.

9. The method of any of claims 1-8, wherein the sample is known or suspected of containing viral nucleic acid from the SIN viral vector.

10. The method of claims 1-9, wherein the sample further comprises a known positive control, optionally wherein said positive control is a viral nucleic acid.

11. A method of quantifying a reverse-transcribed amplicon in a sample, the method comprising:
(a) performing quantitative PCR of DNA from a sample known or suspected of containing viral nucleic acid from a self-inactivating (SIN) viral vector by incubation of DNA from the sample with (i) a forward oligonucleotide primer and a reverse oligonucleotide primer, wherein the primers are specific for a region of reverse-transcribed viral DNA from the SIN viral vector, (ii) an oligonucleotide probe specific for the region of the reverse-transcribed viral DNA, wherein the oligonucleotide probe comprises a detectable moiety; and (iii) a DNA polymerase, wherein:
the quantitative PCR is performed under conditions sufficient for amplification of a reverse-transcriptase-dependent amplicon if the amplicon is present in the sample; and
(b) quantifying an amount of the generated reverse-transcriptase-dependent amplicon by detection of a detectable signal from the detectable moiety.

12. A method of determining viral vector copy number in a sample, the method comprising:
(a) performing quantitative PCR of DNA from a sample known or suspected of containing a viral nucleic acid from a self-inactivating viral vector by incubation of DNA from the sample with (i) a forward oligonucleotide primer and a reverse oligonucleotide primer, wherein the primers are specific for a regions of reverse-transcribed viral DNA from the SIN viral vector, (ii) an oligonucleotide probe specific for the region of the reverse-transcribed viral DNA, wherein the oligonucleotide probe comprises a detectable moiety, and (iii) a DNA polymerase, wherein:
the quantitative PCR is performed under conditions sufficient for amplification of a reverse-transcriptase-dependent amplicon if the amplicon is present in the sample;
(b) quantifying an amount of the generated reverse-transcriptase-dependent amplicon by detection of a detectable signal from the detectable moiety; and
(c) determining viral vector copy number in the sample.

13. The method of claim 11, wherein the reverse-transcribed amplicon is reverse-transcription dependent.

14. The method of any of claims 5-13, wherein the quantitative PCR is real-time PCR.

15. The method of any of claims 5-13, wherein the quantitative PCR is a digital PCR amplification.

16. The method of any of claims 5-13, wherein the quantitative PCR is digital droplet PCR (ddPCR).

17. The method of any of claims 1-10, wherein the SIN viral vector is a retroviral vector.

18. The method of any of claims 1-10, wherein the SIN viral vector is a gamma-retroviral vector.

19. The method of claim 17, wherein the retroviral vector is a lentiviral vector.

20. The method of claim 19, wherein the lentiviral vector is derived from HIV-1.

21. The method of any claims 1-20, wherein the forward oligonucleotide primer comprises a contiguous sequence of nucleotides of the plus strand of a first region that is within the deleted U3 (delU3) of the reverse-transcribed SIN viral vector, and the reverse oligonucleotide primer comprises a contiguous sequence of nucleotides of the minus strand of a second region downstream of the first region in the reverse-transcribed viral vector DNA to make a replicable reverse-transcriptase-dependent amplicon.

22. The method of claim 21, wherein the reverse-transcriptase-dependent amplicon is 50 base pairs to 500 base pairs in size.

23. The method of claim 21 or claim 22, wherein the reverse-transcriptase-dependent amplicon is 100 base pairs to 200 base pairs in size.

24. The method of any of claims 21-23, wherein the first region comprises a U3 attachment sequence.

25. The method of any of claims 21-24, wherein the first region comprises a sequence set forth in any one of SEQ ID NOS: 46-48.

26. The method of any of claims 21-25, wherein the forward primer comprises at least a contiguous portion of any one of SEQ ID NOS: 46-48.

27. The method of any of claims 21-26, wherein the first region comprises the sequence set forth in any one of SEQ ID NOS: 1 and 14-28 or a sequence having at least or about 85, 90, 91, 92, 93, 94, 95, 96, 97, 98, or 99% sequence identity to any one of SEQ ID NOS: 1 and 14-28.

28. The method of any of claims 21-27, wherein the first region is set forth in SEQ ID NO:1.

29. The method of any of claims 21-28, wherein the second region comprises at least a portion of a primer binding site (PBS), an IFN-stimulated response element (ISRE), and/or a Psi (Ψ) packaging sequence of the SIN viral vector.

30. The method of any of claims 21-29, wherein the second region comprises the sequence set forth in any one of SEQ ID NOS: 2 and 29-45 or a sequence having at least or about 85, 90, 91, 92, 93, 94, 95, 96, 97, 98, or 99% sequence identity to any one of SEQ ID NOS: 2 and 29-45.

31. The method of claims 21-30, wherein:
(i) the forward primer comprises a contiguous sequence of nucleotides within the sequence set forth in SEQ ID NO:1 or a sequence having at least or about 85, 90, 91, 92, 93, 94, 95, 96, 97, 98, or 99% sequence identity thereto, and the reverse primer comprises a contiguous sequence of nucleotides within the sequence set forth in SEQ ID NO:2 or a sequence having at least or about 85, 90, 91, 92, 93, 94, 95, 96, 97, 98, or 99% sequence identity thereto;
(ii) the forward primer comprises a contiguous sequence of nucleotides within g the sequence set forth in SEQ ID NO:14 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:29 or a sequence having at least or about 85, 90, 91, 92, 93, 94, 95, 96, 97, 98, or 99% sequence identity thereto;
(iii) the forward primer comprises a contiguous sequence of nucleotides within a the sequence set forth in SEQ ID NO:15 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:30 or a sequence having at least or about 85, 90, 91, 92, 93, 94, 95, 96, 97, 98, or 99% sequence identity thereto;
(iv) the forward primer comprises a contiguous sequence of nucleotides within the sequence set forth in SEQ ID NO:16 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:31 or a sequence having at least or about 85, 90, 91, 92, 93, 94, 95, 96, 97, 98, or 99% sequence identity thereto;
(v) the forward primer comprises a contiguous sequence of nucleotides within the sequence set forth in SEQ ID NO:17 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:32 or a sequence having at least or about 85, 90, 91, 92, 93, 94, 95, 96, 97, 98, or 99% sequence identity thereto;
(vi) the forward primer comprises a contiguous sequence of nucleotides within the sequence set forth in SEQ ID NO:18 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:33 or a sequence having at least or about 85, 90, 91, 92, 93, 94, 95, 96, 97, 98, or 99% sequence identity thereto;
(vii) the forward primer comprises a contiguous sequence of nucleotides within the sequence set forth in SEQ ID NO:19 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:35 or a sequence having at least or about 85, 90, 91, 92, 93, 94, 95, 96, 97, 98, or 99% sequence identity thereto;
(viii) the forward primer comprises a contiguous sequence of nucleotides within the sequence set forth in SEQ ID NO:20 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:36 or a sequence having at least or about 85, 90, 91, 92, 93, 94, 95, 96, 97, 98, or 99% sequence identity thereto;
(ix) the forward primer comprises a contiguous sequence of nucleotides within the sequence set forth in SEQ ID NO:21 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:38 or a sequence having at least or about 85, 90, 91, 92, 93, 94, 95, 96, 97, 98, or 99% sequence identity thereto;
(x) the forward primer comprises a contiguous sequence of nucleotides within the sequence set forth in SEQ ID NO:22 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:39 or a sequence having at least or about 85, 90, 91, 92, 93, 94, 95, 96, 97, 98, or 99% sequence identity thereto;
(xi) the forward primer comprises a contiguous sequence of nucleotides within the sequence set forth in SEQ ID NO:23 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:40 or a sequence having at least or about 85, 90, 91, 92, 93, 94, 95, 96, 97, 98, or 99% sequence identity thereto;
(xii) the forward primer comprises a contiguous sequence of nucleotides within the sequence set forth in SEQ ID NO:24 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:41 or a sequence having at least or about 85, 90, 91, 92, 93, 94, 95, 96, 97, 98, or 99% sequence identity thereto;
(xiii) the forward primer comprises a contiguous sequence of nucleotides within the sequence set forth in SEQ ID NO:25 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:42 or a sequence having at least or about 85, 90, 91, 92, 93, 94, 95, 96, 97, 98, or 99% sequence identity thereto;
(xiv) the forward primer comprises a contiguous sequence of nucleotides within the sequence set forth in SEQ ID NO:26 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:43 or a sequence having at least or about 85, 90, 91, 92, 93, 94, 95, 96, 97, 98, or 99% sequence identity thereto;
(xv) the forward primer comprises a contiguous sequence of nucleotides within the sequence set forth in SEQ ID NO:27 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:44 or a sequence having at least or about 85, 90, 91, 92, 93, 94, 95, 96, 97, 98, or 99% sequence identity thereto; or
(xvi) the forward primer comprises a contiguous sequence of nucleotides within the sequence set forth in SEQ ID NO:28 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:45 or a sequence having at least or about 85, 90, 91, 92, 93, 94, 95, 96, 97, 98, or 99% sequence identity thereto.

32. The method of any of claims 1-31, wherein the forward and reverse oligonucleotide primer are each independently at least 14 nucleotides in length.

33. The method of any of claims 1-32, wherein the forward and reverse oligonucleotide primer are each independently 14-30 nucleotides in length.

34. The method of any of claims 1-33, wherein the forward and reverse oligonucleotide primer are each independently 18-25 nucleotides in length.

35. The method of any of claims 1-34, wherein the forward and reverse oligonucleotide primer are each independently 18-22 nucleotides in length.

36. The method of any of claims 1-35, wherein the reverse-transcriptase-dependent amplicon comprises the sequence set forth in SEQ ID NO:10 or a sequence that has at least 80%, at least 85%, at least 90% or at least 95% sequence identity to SEQ ID NO:10.

37. The method of any of claims 1-36, wherein the reverse-transcriptase-dependent amplicon is set forth in SEQ ID NO:10.

38. The method of any of claims 1-37, wherein the forward oligonucleotide primer comprises a sequence that has at least 80%, at least 85%, at least 90% or at least 95% sequence identity to any one of SEQ ID NOS: 3, 4 or 5.

39. The method of any of claims 1-38, wherein the forward oligonucleotide primer comprises the sequence set forth in any of SEQ ID NOS: 3, 4 or 5.

40. The method of any of claims 1-39, wherein the reverse oligonucleotide primer comprises a sequence that has at least 80%, at least 85%, at least 90% or at least 95% sequence identity to any one of SEQ ID NOS: 6 or 7.

41. The method of any of claims 1-40, wherein the reverse oligonucleotide primer comprises the sequence set forth in any of SEQ ID NOS: 6 or 7.

42. The method of any of claims 1-41, wherein the forward oligonucleotide primer is set forth in SEQ ID NO:3 and the reverse oligonucleotide primer is set forth in SEQ ID NO:6.

43. The method of any of claims 4-42, wherein the oligonucleotide probe is specific for a third region between the first region and the second region of the reverse-transcribed SIN viral vector DNA.

44. The method of any of claims 6-43, wherein the oligonucleotide probe is specific for a sequence present in an R and/or U5 region of the viral LTR of the reverse-transcribed SIN viral vector DNA.

45. The method of any of claims 6-44, wherein the oligonucleotide probe comprises a sequence that has at least 80%, at least 85%, at least 90% or at least 95% sequence identity to any one of SEQ ID NOS: 8 or 9 or is a complement thereof.

46. The method of any of claims 6-45, wherein the oligonucleotide probe comprises the sequences as set forth in SEQ ID NOs 8 or 9 or is a complement thereof.

47. The method of any claims 6-46, wherein the oligonucleotide probe comprises the sequence as set forth in SEQ ID NO 8.

48. The method of any of claims 6-47, wherein the probe further comprises a detectable moiety.

49. The method of any of claims 6-48, wherein the detectable moiety is fluorescent.

50. The method of any of claims 6-49, wherein the detectable moiety is selected from FAM, HEX, FITC, Texas Red, TET, JOE, VIC, NED, TAMRA, ROX, ABY, PET, JUN, LIZ, Cy3, or Cy5.

51. The method of any of claims 1-50, wherein the one or more regions of reverse-transcribed viral vector DNA are not present in a non-self-inactivating viral vector.

52. The method of any of claims 1-51, wherein the one or more regions of reverse-transcribed viral vector DNA are not present in a wild-type virus or the wild-type from which the viral vector is derived.

53. The method of any of claims 1-52, wherein the one or more regions of reverse-transcribed viral vector DNA is only present after reverse-transcription.

54. The method of any of claims 1-53, wherein the reverse-transcriptase dependent amplicon is not produced from viral vector transduction residuals that are episomal in a cell transduced with the viral vector, optionally wherein said residuals are plasmids.

55. The method of any of claims 1-54, wherein the sample comprises one or more cells suspecting of comprising viral nucleic acid integrated in the genome.

56. The method of any of claims 1-55, wherein the sample comprises one more cells transduced with a viral vector.

57. The method of any of claims 4-56, wherein the PCR is a multiplex PCR and further comprises producing a reference amplicon of a reference gene in the same reaction.

58. The method of claim 57, wherein the reference gene is an endogenous gene known to be present in the genome of the cells in the sample.

59. The method of claim 58, wherein the reference gene is known to be present in one or two copies in a diploid genome.

60. The method of any of claims 57-59, wherein the reference gene is selected from hTert, β-actin, GAPDH, ribonuclease P/MRP subunit 30 (RPP30).

61. The method of any of claims 12-40, wherein the viral vector copy number is the copy number or the average copy number of the generated reverse-transcriptase-dependent amplicon per cell in the sample.

62. The method of any of claims 12-40, wherein the viral vector copy number is the copy number or the average copy number of the generated reverse-transcriptase-dependent amplicon per diploid genome.

63. The method of claim 62, wherein the vector copy number per diploid genome is calculated as the ratio between the number of copies of the generated reverse-transcriptase-dependent amplicon and the reference amplicon, optionally multiplied by two.

64. A composition comprising (i) a forward oligonucleotide primer that comprises a contiguous sequence of nucleotides of the plus strand of a first region present within the deleted U3 (delU3) of a reverse-transcribed self-inactivating (SIN) viral vector DNA, and (ii) a reverse oligonucleotide primer that comprises a contiguous sequence of nucleotides of the minus strand of a second region downstream of the first region in the reverse-transcribed viral vector DNA to make a replicable reverse-transcriptase-dependent amplicon.

65. The composition of claim 64, wherein the reverse-transcriptase-dependent amplicon is 100 base pairs to 500 base pairs in size.

66. The composition of claim 64 or claim 65, wherein the reverse-transcriptase-dependent amplicon is 200 base pairs to 400 base pairs in size.

67. The composition of any of claims 64-66, wherein the first region comprises the U3 attachment sequence.

68. The composition of any of claims 64-66, wherein the first region comprises the sequence set forth in any one of SEQ ID NOS: 46-48.

69. The composition of any of claims 64-68, wherein the forward primer comprises at least a contiguous portion of any one of SEQ ID NOS: 46-48.

70. The composition of any of claims 64-69, wherein the first region comprises the sequence set forth in any one of SEQ ID NOS: 1 and 14-28 or a sequence having at least or about 85, 90, 91, 92, 93, 94, 95, 96, 97, 98, or 99% sequence identity to any one of SEQ ID NOS: 1 and 14-28.

71. The composition of any of claims 64-70, wherein the first region is set forth in SEQ ID NO:1.

72. The composition of any of claims 64-71, wherein the second region comprises at least a portion of the primer binding site (PBS), the IFN-stimulated response element (ISRE), and/or the Psi (Ψ) packaging sequence of the SIN viral vector.

73. The composition of any of claims 64-72, wherein the second region comprises the sequence set forth in any one of SEQ ID NOS: 2 and 29-45 or a sequence having at least or about 85, 90, 91, 92, 93, 94, 95, 96, 97, 98, or 99% sequence identity to any one of SEQ ID NOS: 2 and 29-45.

74. The composition of any of claims 64-73, wherein:
(i) the forward primer comprises a contiguous sequence of nucleotides within the sequence set forth in SEQ ID NO:1 or a sequence having at least or about 85, 90, 91, 92, 93, 94, 95, 96, 97, 98, or 99% sequence identity thereto, and the reverse primer comprises a contiguous sequence of nucleotides within the sequence set forth in SEQ ID NO:2 or a sequence having at least or about 85, 90, 91, 92, 93, 94, 95, 96, 97, 98, or 99% sequence identity thereto;
(ii) the forward primer comprises a contiguous sequence of nucleotides within the sequence set forth in SEQ ID NO:14 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:29 or a sequence having at least or about 85, 90, 91, 92, 93, 94, 95, 96, 97, 98, or 99% sequence identity thereto;
(iii) the forward primer comprises a contiguous sequence of nucleotides within the sequence set forth in SEQ ID NO:15 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:30 or a sequence having at least or about 85, 90, 91, 92, 93, 94, 95, 96, 97, 98, or 99% sequence identity thereto;
(iv) the forward primer comprises a contiguous sequence of nucleotides within the sequence set forth in SEQ ID NO:16 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:31 or a sequence having at least or about 85, 90, 91, 92, 93, 94, 95, 96, 97, 98, or 99% sequence identity thereto;
(v) the forward primer comprises a contiguous sequence of nucleotides within the sequence set forth in SEQ ID NO:17 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:32 or a sequence having at least or about 85, 90, 91, 92, 93, 94, 95, 96, 97, 98, or 99% sequence identity thereto;
(vi) the forward primer comprises a contiguous sequence of nucleotides within the sequence set forth in SEQ ID NO:18 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:33 or a sequence having at least or about 85, 90, 91, 92, 93, 94, 95, 96, 97, 98, or 99% sequence identity thereto;
(vii) the forward primer comprises a contiguous sequence of nucleotides within the sequence set forth in SEQ ID NO:19 or a sequence having at least or about 85, 90, 91, 92, 93, 94, 95, 96, 97, 98, or 99% sequence identity thereto and the reverse primer comprises a contiguous sequence of nucleotides within a the sequence set forth in SEQ ID NO:35 or a sequence having at least or about 85, 90, 91, 92, 93, 94, 95, 96, 97, 98, or 99% sequence identity thereto;
(viii) the forward primer comprises a contiguous sequence of nucleotides within the sequence set forth in SEQ ID NO:20 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:36 or a sequence having at least or about 85, 90, 91, 92, 93, 94, 95, 96, 97, 98, or 99% sequence identity thereto;
(ix) the forward primer comprises a contiguous sequence of nucleotides within the sequence set forth in SEQ ID NO:21 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:38 or a sequence having at least or about 85, 90, 91, 92, 93, 94, 95, 96, 97, 98, or 99% sequence identity thereto;
(x) the forward primer comprises a contiguous sequence of nucleotides within the sequence set forth in SEQ ID NO:22 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:39 or a sequence having at least or about 85, 90, 91, 92, 93, 94, 95, 96, 97, 98, or 99% sequence identity thereto;
(xi) the forward primer comprises a contiguous sequence of nucleotides within the sequence set forth in SEQ ID NO:23 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:40 or a sequence having at least or about 85, 90, 91, 92, 93, 94, 95, 96, 97, 98, or 99% sequence identity thereto;
(xii) the forward primer comprises a contiguous sequence of nucleotides within the sequence set forth in SEQ ID NO:24 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:41 or a sequence having at least or about 85, 90, 91, 92, 93, 94, 95, 96, 97, 98, or 99% sequence identity thereto;
(xiii) the forward primer comprises a contiguous sequence of nucleotides within the sequence set forth in SEQ ID NO:25 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:42 or a sequence having at least or about 85, 90, 91, 92, 93, 94, 95, 96, 97, 98, or 99% sequence identity thereto;
(xiv) the forward primer comprises a contiguous sequence of nucleotides within the sequence set forth in SEQ ID NO:26 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:43 or a sequence having at least or about 85, 90, 91, 92, 93, 94, 95, 96, 97, 98, or 99% sequence identity thereto;
(xv) the forward primer comprises a contiguous sequence of nucleotides within the sequence set forth in SEQ ID NO:27 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:44 or a sequence having at least or about 85, 90, 91, 92, 93, 94, 95, 96, 97, 98, or 99% sequence identity thereto; or
(xvi) the forward primer comprises a contiguous sequence of nucleotides within the sequence set forth in SEQ ID NO:28 or a sequence having at least or about 85, 90, 91, 92, 93, 94, 95, 96, 97, 98, or 99% sequence identity thereto and the reverse primer comprises a contiguous sequence of nucleotides within the sequence set forth in SEQ ID NO:45 or a sequence having at least or about 85, 90, 91, 92, 93, 94, 95, 96, 97, 98, or 99% sequence identity thereto.

75. The composition of any of claims 64-74, wherein the forward and reverse oligonucleotide primer are each independently at least 15 nucleotides in length.

76. The composition of any of claims 64-75, wherein the forward and reverse oligonucleotide primer are each independently 15-30 nucleotides in length.

77. The composition of any of claims 64-76, wherein the forward and reverse oligonucleotide primer are each independently 18-25 nucleotides in length.

78. The composition of any of claims 64-77, wherein the forward and reverse oligonucleotide primer are each independently 18-22 nucleotides in length.

79. The composition of any of claims 64-78, wherein the reverse-transcriptase-dependent amplicon comprises the sequence set forth in SEQ ID NO:10 or a sequence that has at least 80%, at least 85%, at least 90% or at least 95% sequence identity to SEQ ID NO:10.

80. The composition of any of claims 64-79, wherein the reverse-transcriptase-dependent amplicon is set forth in SEQ ID NO:10.

81. The composition of any of claims 64-80, wherein the forward oligonucleotide primer comprises a sequence that has at least 80%, at least 85%, at least 90% or at least 95% sequence identity to any one of SEQ ID NOS: 3, 4 or 5.

82. The composition of any of claims 64-81, wherein the forward oligonucleotide primer comprises the sequence set forth in any of SEQ ID NOS: 3, 4 or 5.

83. The composition of any of claims 64-82, wherein the reverse oligonucleotide primer comprises a sequence that has at least 80%, at least 85%, at least 90% or at least 95% sequence identity to any one of SEQ ID NOS: 6 or 7.

84. The composition of any of claims 64-83, wherein the reverse oligonucleotide primer comprises the sequence set forth in any of SEQ ID NOS: 6 or 7.

85. The composition of any of claims 64-84, wherein the forward oligonucleotide primer is set forth in SEQ ID NO:3 and the reverse oligonucleotide primer is set forth in SEQ ID NO:6.

86. The composition of any of claims 64-85, wherein the composition further comprises a polymerase enzyme.

87. The composition of claim 86, wherein the polymerase enzyme is a DNA polymerase, optionally a Taq DNA polymerase, Hot Start DNA polymerase and/or a high fidelity DNA polymerase.

88. The composition of any of claims 64-87, wherein the composition further comprises an oligonucleotide probe.

89. The composition of claim 88, wherein the oligonucleotide probe is specific for a sequence present in an R and/or U5 region of the viral LTR.

90. The composition of claim 88 or claim 89, wherein the oligonucleotide probe comprises a sequence that has at least 80%, at least 85%, at least 90% or at least 95% sequence identity to any one of SEQ ID NOS: 8 or 9 or is a complement thereof.

91. The composition of any of claims 88-90, wherein the probe comprises the nucleic acid sequence set forth in any of SEQ ID NOs 8 or 9.

92. The composition of any of claims 88-91, wherein the probe further comprises a detectable moiety, optionally wherein said moiety is fluorescent.

93. The composition of claim 92, wherein the detectable moiety is selected from FAM, HEX, FITC, Texas Red, TET, VIC, TAMRA, ROX, LIZ, Cy3, or Cy5.

94. A probe, comprising a nucleic acid sequence set forth in any of SEQ ID NOs. 8 or 9.

95. The probe of claim 94, wherein the probe further comprises a detectable moiety.

96. The probe of claim 95, wherein the detectable moiety is fluorescent.

97. The probe of claim 95 or claim 96, wherein the detectable moiety is selected from FAM, HEX, FITC, Texas Red, TET, VIC, TAMRA, ROX, LIZ, Cy3, or Cy5.

98. A kit, comprising the composition of any one of claims 64-93 and/or any one of the probes of claims 94-97.

99. A kit, comprising (i) a forward oligonucleotide primer comprising a nucleic acid sequence set forth in any of SEQ ID NOs 3-5, (ii) a reverse oligonucleotide primer comprising a nucleic acid sequence set forth in any of SEQ ID NOs 6-7, and (iii) a probe comprising a nucleic acid sequence set forth in any of SEQ ID NOs 8 or 9.

100. The kit of claim 99, wherein (i) the forward oligonucleotide primer comprises a nucleic acid sequence set forth in SEQ ID NO. 3, (ii) the reverse oligonucleotide primer comprises a nucleic acid sequence set forth in SEQ ID NO. 7, and (iii) the probe comprises a nucleic acid sequence set forth in SEQ ID NO. 8.

101. A reaction mixture, comprising the composition of any of claims 64-93, wherein the mixture further comprises a sample.

102. The reaction mixture of claim 101, wherein the sample comprises DNA from one more cells transduced with a self-inactivating (SIN) viral vector.

103. The reaction mixture of claim 101 or claim 102, wherein the sample comprises DNA from one or more cells suspecting of comprising SIN viral nucleic acid integrated in the genome.

104. The reaction mixture of any of claims 101-103, wherein the reaction mixture further comprises all four deoxyribonucleoside triphosphates, and/or an appropriate buffer, optionally wherein said buffer comprises Mn+2.


SEQUENCE LISTING (48 sequences)

SEQ ID NO: 1 - DNA, 55 bp, synthetic construct
  source 1..55: mol_type = other DNA
  misc_feature 1..55: note = Region for Forward Primers
tggaagggctaattcactcccaacgaagacaagatctgctttttgcttgtactgg

SEQ ID NO: 2 - DNA, 34 bp, synthetic construct
  source 1..34: mol_type = other DNA
  misc_feature 1..34: note = Region for Reverse Primers
ctgaaagcgaaagggaaaccagagctctctcgac

SEQ ID NO: 3 - DNA, 20 bp, synthetic construct
  source 1..20: mol_type = other DNA
  misc_feature 1..20: note = FWD 1
ggaagggctaattcactccc

SEQ ID NO: 4 - DNA, 21 bp, synthetic construct
  source 1..21: mol_type = other DNA
  misc_feature 1..21: note = FWD 2
cccaacgaagacaagatctgc

SEQ ID NO: 5 - DNA, 21 bp, synthetic construct
  source 1..21: mol_type = other DNA
  misc_feature 1..21: note = FWD 3
gagctcggtacctttaagacc

SEQ ID NO: 6 - DNA, 21 bp, synthetic construct
  source 1..21: mol_type = other DNA
  misc_feature 1..21: note = REV 1
cctgaaagcgaaagggaaacc

SEQ ID NO: 7 - DNA, 21 bp, synthetic construct
  source 1..21: mol_type = other DNA
  misc_feature 1..21: note = REV 2
ggtttccctttcgctttcagg

SEQ ID NO: 8 - DNA, 23 bp, synthetic construct
  source 1..23: mol_type = other DNA
  misc_feature 1..23: note = Probe 1
tgcccgtctgttgtgtgactctg

SEQ ID NO: 9 - DNA, 22 bp, synthetic construct
  source 1..22: mol_type = other DNA
  misc_feature 1..22: note = Probe 2
tgcttgtactgggtctctctgg

SEQ ID NO: 10 - DNA, 273 bp, synthetic construct
  source 1..273: mol_type = other DNA
  misc_feature 1..273: note = DelU3 Amplicon
ctggaagggctaattcactcccaacgaagacaagatctgctttttgcttgtactggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaacc

SEQ ID NO: 11 - DNA, 19 bp, synthetic construct
  source 1..19: mol_type = other DNA
  misc_feature 1..19: note = WPRE FWD
actttcgctttccccctcc

SEQ ID NO: 12 - DNA, 20 bp, synthetic construct
  source 1..20: mol_type = other DNA
  misc_feature 1..20: note = WPRE REV
cggaattgtcagtgcccaac

SEQ ID NO: 13 - DNA, 18 bp, synthetic construct
  source 1..18: mol_type = other DNA
  misc_feature 1..18: note = WPRE Probe
ccacggcggaactcatcg

SEQ ID NO: 14 - DNA, 35 bp, synthetic construct
  source 1..35: mol_type = other DNA
  misc_feature 1..35: note = pHIV-7 (Forward)
tggaagggctaattcactcccaaagaagacaagat

SEQ ID NO: 15 - DNA, 55 bp, synthetic construct
  source 1..55: mol_type = other DNA
  misc_feature 1..55: note = pTNS9 (Forward)
tggaagggctaattcactcccaacgaagacaagatatccttgatctgtggatcta

SEQ ID NO: 16 - DNA, 35 bp, synthetic construct
  source 1..35: mol_type = other DNA
  misc_feature 1..35: note = pWPXL (Forward)
tggaagggctaattcactcccaacgaagacaagat

SEQ ID NO: 17 - DNA, 35 bp, synthetic construct; identical to SEQ ID NO: 16
  source 1..35: mol_type = other DNA
  misc_feature 1..35: note = pRRL (Forward)

SEQ ID NO: 18 - DNA, 35 bp, synthetic construct; identical to SEQ ID NO: 16
  source 1..35: mol_type = other DNA
  misc_feature 1..35: note = pCCL (Forward)

SEQ ID NO: 19 - DNA, 55 bp, synthetic construct; identical to SEQ ID NO: 15
  source 1..55: mol_type = other DNA
  misc_feature 1..55: note = pNL U3 (Forward)

SEQ ID NO: 20 - DNA, 32 bp, synthetic construct
  source 1..32: mol_type = other DNA
  misc_feature 1..32: note = pCSCIGW (Forward)
tggaagggctaattcactcccaaagaagacaa

SEQ ID NO: 21 - DNA, 35 bp, synthetic construct
  source 1..35: mol_type = other DNA
  misc_feature 1..35: note = pRSIEG (Forward)
tggaagggctaattcactcccaacgaagataagat

SEQ ID NO: 22 - DNA, 35 bp, synthetic construct; identical to SEQ ID NO: 16
  source 1..35: mol_type = other DNA
  misc_feature 1..35: note = pHage (Forward)

SEQ ID NO: 23 - DNA, 35 bp, synthetic construct; identical to SEQ ID NO: 16
  source 1..35: mol_type = other DNA
  misc_feature 1..35: note = pSF Lenti (Forward)

SEQ ID NO: 24 - DNA, 35 bp, synthetic construct; identical to SEQ ID NO: 16
  source 1..35: mol_type = other DNA
  misc_feature 1..35: note = TGGAAGGGCTAATTCACTCCCAACGAAGACAAGAT

SEQ ID NO: 25 - DNA, 35 bp, synthetic construct
  source 1..35: mol_type = other DNA
  misc_feature 1..35: note = P156RRLsinppt (Forward)
tggaagggctagctcactcccaacgaagacaagat

SEQ ID NO: 26 - DNA, 22 bp, synthetic construct
  source 1..22: mol_type = other DNA
  misc_feature 1..22: note = pLL3.7m (Forward)
tggaagggctaattcactccca

SEQ ID NO: 27 - DNA, 55 bp, synthetic construct; identical to SEQ ID NO: 15
  source 1..55: mol_type = other DNA
  misc_feature 1..55: note = pLV-GFPSpark (Forward)

SEQ ID NO: 28 - DNA, 55 bp, synthetic construct; identical to SEQ ID NO: 15
  source 1..55: mol_type = other DNA
  misc_feature 1..55: note = pLVX (Forward)

SEQ ID NO: 29 - DNA, 37 bp, synthetic construct
  source 1..37: mol_type = other DNA
  misc_feature 1..37: note = pHIV-7 (Group VI) (Reverse)
ctgaaagcgaaagggaaagcagaggagctctctcgac

SEQ ID NO: 30 - DNA, 37 bp, synthetic construct; identical to SEQ ID NO: 29
  source 1..37: mol_type = other DNA
  misc_feature 1..37: note = pTNS9 (Group III) (Reverse)

SEQ ID NO: 31 - DNA, 37 bp, synthetic construct; identical to SEQ ID NO: 29
  source 1..37: mol_type = other DNA
  misc_feature 1..37: note = pWPXL (Group II) (Reverse)

SEQ ID NO: 32 - DNA, 34 bp, synthetic construct
  source 1..34: mol_type = other DNA
  misc_feature 1..34: note = pRRL(Group I) (Reverse)
ccgaaagcgaaagggaaagcagagctctctcgac

SEQ ID NO: 33 - DNA, 34 bp, synthetic construct; identical to SEQ ID NO: 32
  source 1..34: mol_type = other DNA
  misc_feature 1..34: note = pCCL (Group I) (Reverse)

SEQ ID NO: 34 - DNA, 37 bp, synthetic construct
  source 1..37: mol_type = other DNA
  misc_feature 1..37: note = pTYF (Group VII) (Reverse)
ctgaaagcgaaagtaaaagcagaggagatctctcgac

SEQ ID NO: 35 - DNA, 37 bp, synthetic construct; identical to SEQ ID NO: 34
  source 1..37: mol_type = other DNA
  misc_feature 1..37: note = pNL U3 (Group V) (Reverse)

SEQ ID NO: 36 - DNA, 37 bp, synthetic construct; identical to SEQ ID NO: 29
  source 1..37: mol_type = other DNA
  misc_feature 1..37: note = pCSCIGW (Group II) (Reverse)

SEQ ID NO: 37 - DNA, 37 bp, synthetic construct; identical to SEQ ID NO: 29
  source 1..37: mol_type = other DNA
  misc_feature 1..37: note = pLenti-III (Group A) (Reverse)

SEQ ID NO: 38 - DNA, 37 bp, synthetic construct; identical to SEQ ID NO: 29
  source 1..37: mol_type = other DNA
  misc_feature 1..37: note = pRSIEG (Group C) (Reverse)

SEQ ID NO: 39 - DNA, 37 bp, synthetic construct; identical to SEQ ID NO: 29
  source 1..37: mol_type = other DNA
  misc_feature 1..37: note = pHage (Group A) (Reverse)

SEQ ID NO: 40 - DNA, 37 bp, synthetic construct; identical to SEQ ID NO: 29
  source 1..37: mol_type = other DNA
  misc_feature 1..37: note = pSF Lenti (Group C) (Reverse)

SEQ ID NO: 41 - DNA, 37 bp, synthetic construct; identical to SEQ ID NO: 29
  source 1..37: mol_type = other DNA
  misc_feature 1..37: note = pLenti7.3 (Group C) (Reverse)

SEQ ID NO: 42 - DNA, 37 bp, synthetic construct; identical to SEQ ID NO: 29
  source 1..37: mol_type = other DNA
  misc_feature 1..37: note = P156RRLsinppt (Group C) (Reverse)

SEQ ID NO: 43 - DNA, 37 bp, synthetic construct; identical to SEQ ID NO: 29
  source 1..37: mol_type = other DNA
  misc_feature 1..37: note = pLL3.7m (Group A) (Reverse)

SEQ ID NO: 44 - DNA, 37 bp, synthetic construct; identical to SEQ ID NO: 29
  source 1..37: mol_type = other DNA
  misc_feature 1..37: note = pLV-GFPSpark (Group A) (Reverse)

SEQ ID NO: 45 - DNA, 37 bp, synthetic construct; identical to SEQ ID NO: 29
  source 1..37: mol_type = other DNA
  misc_feature 1..37: note = pLVX (Group A) (Reverse)

SEQ ID NO: 46 - DNA, 27 bp, synthetic construct
  source 1..27: mol_type = other DNA
  misc_feature 1..27: note = U3 att seq 1
tggaagggctaattcactcccaaagaa

SEQ ID NO: 47 - DNA, 27 bp, synthetic construct
  source 1..27: mol_type = other DNA
  misc_feature 1..27: note = U3 att seq 2
tggaagggctaattcactcccaacgaa

SEQ ID NO: 48 - DNA, 27 bp, synthetic construct
  source 1..27: mol_type = other DNA
  misc_feature 1..27: note = U3 att seq 3
tggaagggctagctcactcccaacgaa